(19) United States
(12) Patent Application Publication
(10) Pub. No.: US 2022/0233723 A1
(43) Pub. Date: Jul. 28, 2022

(54) METHOD FOR INCREASING THE RELEASE OF MEDICAL COMPOUNDS FROM NANOPARTICLES BY AN ALTERATION STEP AND A PHYSICO-CHEMICAL DISTURBANCE STEP

(71) Applicant: NANOBACTERIE, Paris (FR)

(72) Inventor: Edouard ALPHANDÉRY, Paris (FR)

(73) Assignee: NANOBACTERIE, Paris (FR)

(21) Appl. No.: 17/605,383

(22) PCT Filed: Apr. 23, 2020
(86) PCT No.: PCT/IB2020/053854

§ 371 Date: Oct. 21, 2021

(30) Foreign Application Priority Data
Apr. 25, 2019 (FR) 1904404

(51) Int. Cl. A61K 49/00; A61K 41/00

(52) U.S. Cl. CPC A61K 49/0002; A61K 41/0052

(57) ABSTRACT

A method for increasing the release of at least one compound, the compound being initially an initial compound bound to at least one initial nanoparticle, the initial compound bound to the initial nanoparticle forming an initial particle, wherein the initial particle includes at least one active ingredient, the method including at least one step of alteration of the initial particle and at least one step of physico-chemical disturbance of an altered particle resulting from the alteration.

### FIELD OF THE INVENTION

The field of the invention is that of a method that enables to increase the quantity of compounds released from a nanoparticle by alteration and/or physico-chemical disturbance of the particle comprising the nanoparticle and the compound.

### TECHNICAL BACKGROUND

When nanoparticles are administered to/in an organism, they have a tendency to be altered, notably when they are internalized in cells or in some cellular compartments such as lysosomes. They can then loose some of their properties such as their heating power and not anymore be efficient for therapeutic treatments, (Di Corato et al, Biomaterials, V. 32, P. 6400 (2014)).

WO 2017/068252 (Nanobacterie) discloses magnetosomes having a luminescent substance attached to the magnetosomes that is dissociated from said magnetosomes after application of a radiation, leading to luminescent intensity increase, without magnetosome alteration.

WO 2011/061259 (Nanobacterie) discloses the use of magnetosomes in the treatment of tumors by heat therapy.

### DESCRIPTION OF THE INVENTION

One of the technical problems to be solved by the present invention is to provide an alternative method for treating a disease such as cancer or virus/viral/bacterial infections via nanoparticles.

The whole prior art is silent about the unexpected and surprising finding by the inventor that the alteration of nanoparticles bound to compounds can under certain conditions increase the release of compounds from these nanoparticles and thus increase or maintain the therapeutic activity of the nanoparticles. This discovery highlights a behavior, which is at the opposite to that expected or generally described in the literature, i.e. the fact that nanoparticle altering conditions undermine or weaken the therapeutic activity of nanoparticles.

One technical effect, which differs from what is expected from the prior art, is an unexpected and surprising increase in the release of compounds comprising an active ingredient, for an altered particle and an altered and disturbed particle, said compounds being able to have a medical effect or to cure an animal or a human being by partial or complete release of these compounds, which preferentially located in or diffuse towards a targeted organ (see FIGS. 7 to 10).

The present invention relates to a method for increasing the release of at least one compound, said compound being initially an initial compound bound to at least one initial nanoparticle, said initial compound bound to said initial nanoparticle forming at least one initial particle, and wherein said initial particle preferentially comprises at least one active ingredient,

and wherein said method comprises at least one of the following steps among a) and b):
a) preferentially altering said initial particle, wherein said altering is associated with modification of at least one property of said initial particle, said altering resulting in formation of an altered particle composed of at least one altered nanoparticle and at least one altered compound,
and wherein said altered particle preferentially comprises at least one active ingredient,
and wherein said altering is preferentially defined as at least one step selected in the group consisting of steps i) to xii):
i) decreasing particle size, preferentially from the size of the initial particle down to the size of the altered particle, where this decrease is such that S_{A}/S_{I} or (S_{I}−S_{A})/S_{I} is between 10⁻³% and 99.99%, where S_{A} and S_{I} are the sizes of the altered and initial particles, respectively,
ii) decreasing a number of compounds bound to the nanoparticle, preferentially from a number nᵢ of initial compounds bound to the initial nanoparticle down to a number nₐ of altered compounds bound to the altered nanoparticle, where nᵢ/nₐ is between 1 and 10¹⁰,
iii) decreasing a binding strength of least one bond between the compound and the nanoparticle, preferentially from a binding strength Sᵢ of at least one initial bond between the initial compound and the initial nanoparticle to a binding strength Sₐ of at least one altered bond between the altered compound and the altered nanoparticle,
iv) breaking at least one bond between the altered compound and the altered nanoparticle,
v) decreasing a bond-dissociation energy between the compound and the nanoparticle, preferentially from a bond-dissociation energy E_{di} between the initial compound and the initial nanoparticle down to a bond-dissociation energy E_{da} between the altered compound and the altered nanoparticle,
vi) decreasing a coating thickness of the nanoparticle, preferentially from a coating thickness CTᵢ of the initial nanoparticle down to a coating thickness CTₐ of the altered nanoparticle,
vii) decreasing a percentage in mass of organic material or carbon or carbonaceous material of the altered particle, preferentially compared with the percentage in mass of organic material or carbon or carbonaceous material of the initial particle,
viii) decreasing cluttering of the compound bound to the nanoparticle, preferentially from a large cluttering of the initial compound bound to the initial nanoparticle down to a small cluttering of the altered compound bound to the altered nanoparticle,
ix) decreasing a number or a concentration of compounds N₁ that prevent the release of compounds N₂ from the nanoparticle, preferentially from a number of initial compounds N₁ᵢ that prevent the release of initial compounds N₂ᵢ from the initial nanoparticle down to a number of altered compounds N₁ₐ that prevent the release of altered compounds N₂ₐ from the altered nanoparticle,
x) inactivating, attenuating, destroying a cell, part of a cell, a virus, part of a virus, a bacterium, and/or part of a bacterium, preferentially from an initial cell, part of an initial cell, an initial virus, part of an initial virus, an initial bacterium, and/or part of an initial bacterium that is/are not inactivated, not attenuated, and/or not destroyed by or in the presence of the initial nanoparticle to an altered cell, part of an altered cell, an altered virus, part of an altered virus, an altered bacterium, and/or part of an altered bacterium that is/are inactivated, attenuated, and/or destroyed by or in the presence of the altered nanoparticle or of the nanoparticle that transforms itself from the initial to the altered nanoparticle,
xi) presenting, processing and/or exposing an antigen or part of an antigen such as an epitope by or in the presence of the altered nanoparticle, preferentially from an initial antigen or part of an initial antigen that is not presented, not processed and/or not exposed by or in the presence of the initial nanoparticle to an altered antigen or part of an altered antigen that is presented, processed and/or exposed by or in the presence of the altered nanoparticle,
and
xii) coating, binding, and/or assembling a nanoparticle by or with a cell, part of a cell, a virus, part of a virus, a bacterium, part of a bacterium, an antigen, and/or part of an antigen, preferentially from an initial nanoparticle that is not coated, bound, and/or assembled by or with an initial cell, part of an initial cell, an initial virus, part of an initial virus, an initial bacterium, part of an initial bacterium, an initial antigen, and/or part of an initial antigen to an altered nanoparticle that is coated, bound, and/or assembled by or with an altered cell, part of an altered cell, an altered virus, part of an altered virus, an altered bacterium, part of an altered bacterium, an altered antigen, and/or part of an altered antigen,
and wherein said alteration preferentially results in xiii) and/or xiv):
xiii) a first partial release generating a first part of altered compounds released from the altered nanoparticle, where the first partial release is due to the complete breaking of the bond between said altered nanoparticle and the first part of said altered compound, and/or
xiv) an absence of release generating a second part of altered compounds that remain bound to the altered nanoparticle, where the absence of release is due to the absence of breaking of the altered bond between said altered nanoparticle and said second part of altered compound,
and wherein the first part and second part of altered compounds preferentially originate from the initial particle, and the sum of said first part and second part of said altered compounds preferentially represent the total number of initial compounds bound to said initial nanoparticle,
and wherein preferentially said alteration, which is applied on the first transforming particle transforming from the initial particle to the altered particle, is carried out in at least one of the following conditions among xv) to xx): xv) by a first internalization of the first transforming particle in a cell, a virus, a bacterium, preferentially in a compartment of a cell, a virus, or a bacterium, preferentially in a lysosome or endosome, xvi) by a first variation of the pH of the first transforming particle or of its environment, preferentially by bringing the pH of the first transforming particle or of its environment to a first acidic pH, xvii) by a first variation of temperature of the first transforming particle or of its environment, preferentially by a first temperature increase of the first transforming particle or of its environment, preferentially by a first temperature variation of the first transforming particle or of its environment by at least 10⁻³° C. above the physiological temperature or above 37° C. or above the temperature of the initial particle, xviii) by bringing the first transforming particle in the presence of altering biological or chemical material, xix) by applying a first radiation on the first transforming particle, and xx) by a first variation the environment of the first transforming particle,
b) preferentially applying a physico-chemical disturbance on said altered particle, preferentially resulting in the formation of an altered and disturbed particle, and preferentially wherein said altered and disturbed particle comprises at least one active ingredient, and preferentially wherein step b) is associated with xxi), xxii), or xxiii):
xxi) an absence of release generating non-released altered and disturbed compounds belonging to group 1 of the second part, which originates from the second part of altered compounds not released by alteration, preferentially at step a)xiii), wherein the absence of release is due to the absence of breaking of the altered and disturbed bond between the altered and disturbed nanoparticle and the group 1 of the second part of altered and disturbed compounds,
xxii) a second partial release generating released altered and disturbed compounds belonging to group 2 of the second part, which originates from the second part of altered compounds not released by alteration, preferentially at step a)xiii), wherein said second partial release is due to the complete breaking of the bond between the group 2 of the second part of the altered and disturbed compounds and the altered and disturbed nanoparticle, or
xxiii) a second total release of altered and disturbed compounds, which originate from the second part of altered compounds not released by alteration, preferentially at step a)xiii), wherein said second total release is due to the complete breaking of the bond between all said altered and disturbed compounds and said altered and disturbed nanoparticle,
and wherein preferentially said physico-chemical disturbance which is applied on the second transforming particle transforming from the altered particle to the altered and disturbed particle, is carried out in at least one of the following conditions among xxiv) to xxix): xxiv) by a second internalization of the second transforming particle in a cell, a virus, a bacterium, preferentially in a compartment of a cell, a virus, or a bacterium, preferentially in a lysosome or endosome, xxv) by a second variation of the pH of the second transforming particle or of its environment, preferentially by bringing the pH of the second transforming particle or of its environment to a second acidic pH, xxvi) by a second variation of temperature of the second transforming particle or of its environment, preferentially by a second temperature increase of the second transforming particle or of its environment, preferentially by a second temperature variation of the second transforming particle or of its environment by at least 10⁻³° C. above the physiological temperature or above 37° C. or above the temperature of the altered particle, xxvii) by bringing the second transforming particle in the presence of an altering biological or chemical material, xxviii) by applying a second radiation on the second transforming particle, and xxix) by a second variation the environment of the second transforming particle.

The present invention also relates to a method comprising at least one of the following steps:

i) inactivating, attenuating, destroying a cell, part of a cell, a virus, part of a virus, a bacterium, and/or part of a bacterium, preferentially from an initial cell, part of an initial cell, an initial virus, part of an initial virus, an initial bacterium, and/or part of an initial bacterium that is/are not inactivated, not attenuated, and/or not destroyed by or in the presence of the initial nanoparticle to an altered cell, part of an altered cell, an altered virus, part of an altered virus, an altered bacterium, and/or part of an altered bacterium that is/are inactivated, attenuated, and/or destroyed by or in the presence of the altered nanoparticle or of the nanoparticle that transforms itself from the initial to the altered nanoparticle,
ii) presenting, processing and/or exposing an antigen or part of an antigen such as an epitope by or in the presence of the altered nanoparticle, preferentially from an initial antigen or part of an initial antigen that is not presented, not processed and/or not exposed by or in the presence of the initial nanoparticle to an altered antigen or part of an altered antigen that is presented, processed and/or exposed by or in the presence of the altered nanoparticle, and
iii) coating, binding, and/or assembling a nanoparticle by or with a cell, part of a cell, a virus, part of a virus, a bacterium, part of a bacterium, an antigen, and/or part of an antigen, preferentially from an initial nanoparticle that is not coated, bound, and/or assembled by or with an initial cell, part of an initial cell, an initial virus, part of an initial virus, an initial bacterium, part of an initial bacterium, an initial antigen, and/or part of an initial antigen to an altered nanoparticle that is coated, bound, and/or assembled by or with an altered cell, part of an altered cell, an altered virus, part of an altered virus, an altered bacterium, part of an altered bacterium, an altered antigen, and/or part of an altered antigen, and wherein during step i), ii) and/or iii), at least one compound is not released from the nanoparticle, and wherein step i), ii) and/or iii) preferentially result(s) from or is/are associated with alteration and/or physico-chemical disturbance.

The invention also relates to a method for increasing the release of at least one compound, said compound being initially an initial compound bound to at least one initial nanoparticle, said initial compound bound to said initial nanoparticle forming at least one initial particle,
and wherein said initial particle preferentially comprises at least one active ingredient,
and wherein said method comprises at least one step among a) and b):
a) altering said initial particle, wherein altering is associated with modification of at least one property of said initial particle,
wherein said altering preferentially results in the formation of an altered particle comprising at least one altered nanoparticle and at least one altered compound,
wherein said altered particle preferentially comprises at least one active ingredient, wherein said altering preferentially results in i) and ii):
i) a first partial release generating a first part of altered compounds released from the altered nanoparticle, where the first partial release is due to the complete breaking of the bond between said altered nanoparticle and the first part of said altered compound,
and
ii) an absence of release generating a second part of altered compounds that remain bound to the altered nanoparticle, where the absence of release is due to the absence of breaking of the altered bond between said altered nanoparticle and said second part of altered compound,
and wherein the first part and second part of altered compounds preferentially originate from the initial particle, and the sum of said first part and second part of said altered compounds represent the total number of initial compounds bound to said initial nanoparticle,
b) applying a physico-chemical disturbance on said altered particle, preferentially resulting in the formation of an altered and disturbed particle,
and wherein said altered and disturbed particle preferentially comprises at least one active ingredient,
and wherein step b) is preferentially associated with at least one step among iii), iv) and v):
iii) an absence of release generating non-released altered and disturbed compounds belonging to group 1 of the second part, which preferentially originates from the second part of altered compounds not released by alteration, preferentially at step a)i), wherein the absence of release is preferentially due to the absence of breaking of the altered and disturbed bond between the altered and disturbed nanoparticle and the group 1 of the second part of altered and disturbed compounds,
iv) a second partial release generating released altered and disturbed compounds belonging to group 2 of the second part, which preferentially originates from the second part of altered compounds not released by alteration, preferentially at step a)i), wherein said second partial release is preferentially due to the complete breaking of the bond between the group 2 of the second part of the altered and disturbed compounds and the altered and disturbed nanoparticle,
and
v) a second total release of altered and disturbed compounds, which preferentially originate from the second part of altered compounds not released by alteration, preferentially at step a)ii),
wherein said second total release is preferentially due to the complete breaking of the bond between all said altered and disturbed compounds and said altered and disturbed nanoparticle.

The present invention also relates to a method for obtaining an altered and disturbed particle comprising at least one step among α), β), γ), and η):
α) applying an alteration on at least one initial particle comprising at least one initial nanoparticle and at least one releasable initial compound, which is initially bound to said initial nanoparticle via an initial bond,
β) obtaining at least one altered particle, comprising at least one altered nanoparticle and at least one releasable altered compound, where a first partial release generates the release of a first part of altered compound during the alteration, said altered compounds being preferentially divided between:
i) a first part of altered compounds comprising altered compounds released from the altered nanoparticle, and
ii) a second part of altered compounds comprising altered compounds bound to the altered nanoparticle via an altered bond,
γ) applying a physico-chemical disturbance on said altered particle,
η) obtaining at least one altered and disturbed particle, comprising at least one altered and disturbed nanoparticle and at least one releasable altered and disturbed compound, where a second partial release generates the release of a second part of altered and disturbed compounds during physico-chemical disturbance, said altered and disturbed compounds being preferentially divided between:
i) group 1 of second part of altered and disturbed compounds comprising altered and disturbed compounds bound to the altered and disturbed nanoparticle via an altered and disturbed bond, and
ii) group 2 of second part of altered and disturbed compounds comprising altered and disturbed compounds released from the altered and disturbed nanoparticle,
wherein the said initial particle, altered particle, and/or altered and disturbed particle preferentially comprise at least one active ingredient.

The invention also relates to the method according to the invention, wherein step a) of the method comprises:

a) an absence of modification of at least one property of said compound, preferentially an absence of: i) size-reduction of the compound, ii) weakening or destruction of the medical, diagnostic, cosmetic, medical device, or drug activity of the compound, and iii) change of the composition of the compound,
b) an absence of activation of the compound, preferentially of the first part of the compound, and/or
c) an absence of activation of the nanoparticle.

The invention also relates to the method according to the invention, wherein step b) comprises:

a) an absence of modification of at least one property of said altered nanoparticle, preferentially
i) a decrease in size of said nanoparticle during the physico-chemical disturbance application of less than 99% of the initial size of the nanoparticles before step a) and/or step b), ii) a decrease in thickness of the coating of said nanoparticle during the physico-chemical disturbance application by a factor of less than 10³, or iii) a decrease in the percentage in mass of organic material or carbon of the said nanoparticle by a factor of less than 10³,
b) an absence of modification at least one property of said compound, preferentially an absence of: i) size-reduction of the compound, ii) weakening or destruction of the medical, diagnostic, cosmetic, medical device, or drug activity of the compound, or iii) change of the composition of the compound,
c) an absence of activation of the compound, preferentially of the second part of the compound, and/or
d) an absence of activation of the nanoparticle.

The invention also relates to the method according to the invention, wherein the application of the physico-chemical disturbance of step b) on the said altered nanoparticles produces or leads to the activation of:

a) the altered nanoparticles, preferentially by heating the altered nanoparticles or by inducing the release of reactive species from the altered nanoparticles, and/or
b) the compound, preferentially by letting the compound diffuse preferentially in the environment of the nanoparticle or towards the infected body part or towards cells that produce antibodies or by having the compound destroy at least one pathological cell, preferentially directly, i.e. preferentially through the direct destruction by the compound of the pathological cell, or preferentially indirectly, i.e. preferentially by having the compound activating an entity such as a T or B or APC cell or antibody or antigen or antibiotic that is involved in pathological cell destruction.

The invention also relates to the method according to the invention, wherein the bonds between said nanoparticle and said compound are characterized by at least one of the following properties:

a) the bonds between the nanoparticle and the compound releasable by the application of the physico-chemical disturbance of step b) are different from the bonds between the nanoparticle and the compound releasable by the alteration of step a),
b) the bonds between the first part of compound of step a) and the nanoparticle are breakable by alteration of the nanoparticles, and/or
c) the bonds between the second part of compound of step b) and the nanoparticle are breakable by application of the physico-chemical disturbance on the nanoparticle.

The invention also relates to a method for increasing the release of at least one compound from a nanoparticle by following at least one step among steps a) and b), where step a) consists in altering the particle comprising the compound and nanoparticle and step b) consists in applying a physico-chemical disturbance on the particle.

Preferably, the method for increasing the release of at least one compound comprises at least one of the following steps:

step a) comprising the activation of the first part of the altered compound released from the altered nanoparticle by breaking of the initial bond or following the breaking of the initial bond, and/or
step b) comprising the activation of the second part of the altered compound bound to the altered nanoparticle via an altered bond.

In some cases, a(the) step of the method or at least one step of the method according to the invention can designate more than 1, 2, 3, 4, 5, 10, 10³ or 10⁵ step(s) of the method, preferentially identical or different step(s) of the method.

In some other cases, a(the) step of the method or at least one step of the method can designate less than 10⁵⁰, 10¹⁰, 10⁵, 10³, 10, 5, 4, 3, 2 or 1 step(s) of the method, preferentially identical or different step(s) of the method.

In some cases, the step(s) of the method can be repeated more than 1, 2, 3, 4, 5, 10, 10³ or 10⁵ time(s).

In some other cases, the step(s) of the method can be repeated less than 10⁵, 10³, 10, 5, 4, 3, 2 or 1 time(s).

One difference between the prior art and the present invention is applying the successive steps of a) an alteration step leading to an altered particle and b) a physico-chemical disturbance step leading to an altered and disturbed particle.

In one embodiment of the invention, the method according to the invention increases or enables the release of at least one compound from at least one nanoparticle. In some cases, this can mean that: i), initially, i.e. preferentially before or without the method or without at least one step of the method according to the invention, the compound being the initial compound is bound to the initial nanoparticle, ii) during or following or with the method, preferentially step a) of the method, the compound being the altered compound is released from nanoparticle being the altered nanoparticle by alteration as defined in the invention, and/or iii) during or following or with the method, preferentially step(s) a) and/or b) of the method, the compound being the altered and disturbed compound is released from the nanoparticle being the altered and disturbed nanoparticle by alteration and physico-chemical disturbance as defined in the invention.

In some cases, the release of the compound from the nanoparticle can be the dissociation or separation of the compound from the nanoparticle. The release of the compound from the nanoparticle can be the same as the release of the nanoparticle from the compound. The release of the compound from the nanoparticle can be designated as the release of the compound or as the release. The release can also be the isolation, the diffusion, preferentially of the compound, preferentially from or away or at some distance from the nanoparticle, preferentially towards the infected body part or towards an immune region where the compound can activate an immune cell against the infected body part, e.g. through the production of antibodies by such cells or by triggering the diffusion of immune cells such as T cells towards the infected body part or by activating immune cells such as T, B, or APC (antigen presenting) cells or immune entities such as antibodies or cytokines or interleukins or MHC (major histocompatibility complex) or CD (cluster of differentiation) against the infected body part, preferentially so that these cells or entities destroy the infected body part, preferentially so that the compound can control the activity of the immune entities or immune cells to prevent these entities/cells from over-reacting or from creating an auto-immune response or from generating a response that would kill or affect the individual, for example by being too strong or by destroying a too large number of healthy cells or by stopping or weakening the activity of an organ such as the heart.

In one embodiment of the invention, the release of the compound from the nanoparticle is, corresponds to, is associated with: i) the release of the compound from surface or coating or central part or crystallized part or amorphous part or organic part or mineral part of the nanoparticle, ii) an increase of the concentration or number of compounds comprised in the supernate or surrounding or environment of the nanoparticle or nanoparticle suspension or altering medium or body part, preferentially infected body part, or region of the body part not comprising the compounds before the release of the compound and comprising the compound after the release of the compound or a region or location or position located at a distance from the nanoparticles, which is in some cases larger than 10⁻⁵, 0.1, 1, 5, 10, 10², 10³ or 10⁵ nm, which is in some other cases lower than 10⁵, 10³, 10², 5, 2 or 1 nm, iii) the breaking or weakening or alteration of the bond or interaction between the compound and the nanoparticle, or iv) the transition from a state in which the compound is in contact or in interaction with the nanoparticle or bound to the nanoparticle to a state in which the compound is not in contact or not in interaction with the nanoparticle or not bound to the nanoparticle, where these two states can preferentially be observed at any given time before, during, or after the method according to the invention.

In one embodiment of the invention, the compound is released from the nanoparticle when: i) the particle comprises the compound before the release and does not comprise the compound after the release or the nanoparticle is bound to the compound before the release and is not bound to the compound after the release, ii) the distance between the compound and the nanoparticle increases by a factor of at least 1.001, 1.1, 1.2, 1.5, 2, 5, 10, 10³, 10⁵ or 10¹⁰ between before and after the release, iii) the distance between the compound and the nanoparticle increases from less than 10¹⁰, 10⁵, 10³, 10, 5, 2, 1 or 10⁻¹ nm before the release to more than 10⁻⁵, 10⁻³, 10⁻¹, 1, 10 or 10³ nm after the release, or iv) there is a bond between the compound and the nanoparticle before the release while there is no bond or a weaker bond between the compound and the nanoparticle after the release.

According to the invention, the compound is said to dissociate from the nanoparticle(s) or to be released from the nanoparticle(s) when it separates from the nanoparticle(s) to end up in the environment of the nanoparticle(s).

In one embodiment of the invention, the compound is initially bound to the nanoparticle when the compound is not released from the nanoparticle, preferentially before or without alteration of the nanoparticle, and/or preferentially before or without application of a physico-chemical disturbance on the nanoparticle.

In one embodiment of the invention, the release is, is associated with, corresponds to, or results in: i) the release of at least one compound from at least one nanoparticle, ii) the detachment of the compound from the nanoparticle, iii) the breaking or disappearance of the bond between the compound and the nanoparticle, and/or iv) the diffusion of the compound in the environment of the particle.

In some cases, the environment of the particle can be the infected body part.

In one embodiment of the invention, the method for enabling or increasing the release of the compound from the nanoparticle, is a method that increases the quantity or concentration of compounds released from the nanoparticles in at least one of the following manner: i), by a factor of at least 1.001, 1.1, 1.5, 2, 5, 10, 10³, 10⁵ or 10¹⁰, between before and after the method or at least one step of the method, ii) from less than 10¹⁰⁰, 10⁵⁰, 10¹⁰, 10⁵, 10³, 10, 5, 2 or 1 released compounds, preferentially per nanoparticle, before using the method or at least one step of the method, to more than 1, 5, 10, 10³, 10⁵, 10¹⁰, 10²⁰ or 10⁵⁰ released compounds, preferentially per nanoparticle, after using the method or at least one step of the method.

In some cases, alteration is applied on a particle, preferentially the initial particle, preferentially during step α) or a) of the method.

In some cases, applying an alteration on the initial particle can mean that the particle, preferentially the initial particle, is exposed to alteration, or is altered, or is changing its condition from being an initial particle to being an altered particle, preferentially by or following alteration.

In some cases, the alteration can be or be designated as the degradation or the killing or the weakening or the modification or the inactivation or the destruction, of preferentially: i) the particle, ii) the nanoparticle, iii) the compound, or iv) the activity, strength or power of the particle, nanoparticle or compound.

In some cases, the alteration is the change of the state or condition of the particle from an initial state or initial condition of the particle in which the particle is the initial particle to an altered state or altered condition of the particle in which the particle is the altered particle.

In some cases the alteration can be the alteration of the initial particle, the alteration of the altered particle, and/or the alteration of the altered and disturbed particle.

In some cases, the altering or degrading medium can be the environment, matrix, body part that preferentially: i) surrounds, envelops, or embeds the particle, and/or ii) yields, produces, induces, or triggers the alteration of the particle.

The invention also relates to the method according to the invention, wherein the degrading medium is a body part of an individual.

In one embodiment of the invention, the alteration is or results in or is associated with the first step or step a) of the method.

In one embodiment of the invention, the alteration is or results in or is associated with the alteration of the initial particle preferentially resulting in or in the formation of an altered particle. In some cases, the altered particle can comprise at least one altered nanoparticle and/or at least one altered compound.

In one embodiment of the invention, the alteration is or results in or is associated with a modification of at least one property of said initial particle, where each modification can be a sub-step of step a) of the method.

In one embodiment of the invention, the alteration is or results in or is associated with a step or a sub-step of step a).

In one embodiment of the invention, the alteration is or is associated with or corresponds to or leads to or results in: a change or modification of at least one property of the particle, preferentially existing or measured between before, during and/or after alteration.

In one embodiment of the invention, the alteration of the particle, preferentially of the initial particle, is the exposure or mixing of the particle to/with a medium or environment, preferentially of the particle, which lead(s) to, results in, produces, and/or is associated with the alteration of the particle.

In one embodiment of the invention, the alteration is or results in or is associated with one of the mechanisms (first mechanism) that can lead to the release, preferentially the partial release, of the compound from the nanoparticle. A second mechanism that can lead to the release of the compound from the nanoparticle is the application of the physico-chemical disturbance on the nanoparticle. In some cases, the release of the compound from the nanoparticle by the second mechanism can only be achieved or is more efficient when the first mechanism by alteration has occurred preferentially before the physico-chemical disturbance is applied.

In one embodiment, the alteration is, or results in, or produces or is associated with the sub-step of step a) or the modification of a least one property of the initial particle.

In one embodiment of the invention, the alteration is, or results in, or produces or is associated with at least one property selected from the group consisting of i) to xiii):

i) the modification, preferentially the decrease, of the size of the particle,
ii) the modification, preferentially the decrease, of the number of compounds bound to the nanoparticle,
iii) the modification, preferentially the decrease, of the strength of at least one bond between the compound and the nanoparticle,
iv) the breaking of at least one bond between the altered compound and the altered nanoparticle,
v) the modification, preferentially the decrease, of the bond-dissociation energy between the compound and the nanoparticle,
vi) the modification, preferentially the decrease, of the coating thickness of the nanoparticle,
vii) the modification, preferentially the decrease, of the cluttering of the compound bound to the nanoparticle,
viii) the modification, preferentially the decrease, of the number or concentration of compounds N₁ that prevent the release of compounds N₂ from the nanoparticles,
ix) the modification of the chemical composition of the particle,
x) the modification such as the increase or the decrease, of the surface charge or zeta potential, of the particle,
xi) the modification, preferentially the decrease, of the isoelectric point of the particle,
xii) the modification, preferentially the decrease, of the mass or weight of the particle,
xiii) the modification of the magnetic properties of the particle,
xiv) the modification of the properties of assembly, organization, and/or distribution of the nanoparticles, such as their arrangement in chains or aggregates.
xv) the modification of the crystallinity of the particle,
xvi) the modification of the number or concentration of nanoparticles,
xvii) the modification of the morphology or geometry of the nanoparticles,
xviii) the modification of the number of facets of the nanoparticles,
xix) the modification, preferentially the increase, of the faculty to release at least one compound from the particle,
xx) the modification, preferentially the decrease, of the quantity of heat produced by the particle, preferably under the application of a radiation,
xxi) the modification, preferentially the increase, of the quantity of radical or reactive species produced by the nanoparticles, preferably under the application of a radiation,
xxii) the inactivation or attenuation of the compound, preferentially the inactivation or attenuation of a virus or of part of a virus, and
xxiii) the exposure or activation of the compound, preferentially to favor its presentation or interaction for example to or with an immune cell.

In one embodiment of the invention, the alteration is or results in or is associated with a decrease in particle size, preferentially from the size of the initial particle down to the size of the altered particle, where this decrease is preferentially such that S_{A}/S_{I} or (S_{I}−S_{A})/S_{I} is between 10⁻³% and 99.99%, where S_{A} and S_{I} are the sizes of the altered and initial particles, respectively.

In some cases, S_{A}/S_{I} or (S_{I}−S_{A})/S_{I} can be larger than 10⁻⁵⁰, 10⁻²⁰, 10⁻¹⁰, 10⁻⁵, 10⁻¹, 1, 5, 10, 50, 10², 10³, 10⁵ or 10¹⁰%.

In some other cases, S_{A}/S_{I} or (S_{I}−S_{A})/S_{I} can be lower than 10⁵⁰, 10²⁰, 10¹⁰, 10⁵, 10, 5, 2, 1, 10⁻³, 10⁻⁵, 10⁻¹⁰ or 10⁻⁵⁰%.

In still some other cases, S_{A}/S_{I} or (S_{I}−S_{A})/S_{I} can be between 10⁻⁵⁰ and 10⁵⁰%, between 10⁻³ and 10³%, between 10⁻³ and 99.99%, between 10⁻² and 99%, between 10⁻¹ and 90%, between 1 and 85%, or between 5 and 60%.

In one embodiment of the invention, the alteration is or results in or is associated with a decrease in particle size from the size of the initial particle larger than 10⁻³, 0.1, 1, 5, 10 or 100 nm, down to the size of the altered particle smaller than 10⁵, 10³, 100, 50, 20, 10, 5, 3, 2 or 1 nm.

In another embodiment of the invention, the alteration is or results in or is associated with a decrease in particle size from the size of the initial particle to the size of the altered particle by a quantity S_{I}−S_{A}, which is larger than 10⁻⁵, 10⁻¹, 1, 5, 10 or 10³ nm.

In another embodiment of the invention, the alteration is or results in or is associated with a decrease in particle size from the size of the initial particle to the size of the altered particle by a quantity S_{I}−S_{A}, which is smaller than 10⁵, 10, 5, 1 or 10⁻¹ nm.

In one embodiment of the invention, the alteration is associated with, corresponds to, results in, or leads to a size-reduction of the nanoparticle, where the size reduction of the nanoparticle is preferentially a decrease in size of the nanoparticle, which is due to alteration or occurs or is measured during alteration or between before and after alteration.

In some cases, the size-reduction of the nanoparticle can be larger than 10⁻⁵⁰, 10⁻¹⁰, 10⁻⁵, 10⁻¹, 1, 5, 10, 50, 70, 80, 90, 95, 99, 100, 10², 10⁵, 10¹⁰ or 10¹⁰⁰%.

In some cases, the percentage of size reduction resulting from alteration can be equal to (S_{NBA}−S_{NAA})/S_{NBA}, S_{NAA}/S_{NBA}, or (S_{NAA}−S_{NBA})/S_{NBA}, where S_{NBA} and S_{NAA} are the sizes of the nanoparticle before and after alteration, respectively.

In another embodiment of the invention, the size-reduction of the nanoparticle is smaller than 10¹⁰⁰, 10⁵⁰, 10¹⁰, 10⁵, 100, 99.9, 99, 95, 90, 80, 70, 50, 30, 20, 10, 5, 2, 1 or 10⁻³%,

In still another embodiment of the invention, the size-reduction of the nanoparticle is between 10⁻¹⁰⁰ and 10¹⁰⁰%, or between 10⁻⁵ to 99.9%.

In another embodiment of the invention, the alteration is associated with, corresponds to, results in, or leads to the decrease of the number of compounds attached or bound to the nanoparticle, preferentially initially the initial nanoparticle, preferentially in the following manner: i) by a factor of at least 1.001, 1.1, 1.5, 2, 5, 10, 10³, 10⁵, 10¹⁰, or ii) from more than 1, 5, 10, 10³, 10⁵, 10¹⁰ or 10⁵⁰ initial compound(s), preferentially per initial nanoparticle, attached or bound to the initial nanoparticle before alteration to less than 10⁵⁰, 10¹⁰, 10⁵, 10³, 10, 5 or 1 altered compound(s), preferentially per altered nanoparticle, preferentially attached or bound to the altered nanoparticle during or after alteration.

In still another embodiment of the invention, the alteration is, results in, or is associated with a size-reduction of the nanoparticle down to a size that is such that at least one compound remains attached or bound to at least one nanoparticle.

In still another embodiment of the invention, the alteration is a size-reduction of the nanoparticles down to a threshold size, preferentially a threshold size of the altered nanoparticle.

In some cases, the threshold size can be the size that is such that at least one compound remains attached or bond to at least one nanoparticle, preferentially altered nanoparticle. Preferentially, above the threshold size, at least one compound remains or is bound to the nanoparticle, preferentially the altered nanoparticle, while below the threshold size no compound is bound to the nanoparticle, preferentially the altered nanoparticle.

In still some other cases, the threshold size can be the size that is such that between above and below the threshold size, the number of compounds, preferentially altered compounds, bound to the nanoparticle, preferentially altered nanoparticle, is lower, preferentially by: i) a factor of more than 1.1, 1.2, 1.5, 2, 5, 10, 10³ or 10⁵ or ii) more than 1, 2, 3, 5, 10, 10³ or 10⁵ compound(s), preferentially altered compound(s), preferentially per nanoparticle, most preferentially per altered nanoparticle.

In still some other cases, a size that is above the threshold size is a size that is at least 10⁻⁵, 10⁻¹, 1, 5, 10, 10³, 10⁵ or 10¹⁰ nm above the threshold size.

In still some other cases, a size that is below the threshold size is a size that is at least 10⁻⁵, 10⁻¹, 1, 5, 10, 10³, 10⁵ or 10¹⁰ nm below the threshold size In one embodiment of invention, the threshold size is larger than 10⁻¹⁰, 10⁻⁵, 10⁻³, 10⁻¹, 1, 10, 10³, 10⁵ or 10¹⁰ nm.

In another embodiment of the invention, the threshold size is smaller than 10¹⁰, 10⁵, 10³, 10, 5, 2, 1, 10⁻¹ or 10⁻³ nm.

In still another embodiment of the invention, the threshold size is between 10⁻⁵ and 10¹⁰, between 10⁻¹ and 10⁵ nm, or between 10⁻¹ and 10 nm.

In one embodiment of the invention, the alteration is or results in or is associated with an increase in particle size, preferentially from the size of the initial particle up to the size of the altered particle, where this increase is preferentially such that S_{I}/S_{A} or (S_{A}−S_{I})/S_{A} is between 10⁻³% and 99.99%, where S_{A} and S_{I} are the sizes of the altered and initial particles, respectively.

In some cases, S_{I}/S_{A} or (S_{A}−S_{I})/S_{A} can be larger than 10⁻⁵⁰, 10⁻²⁰, 10⁻¹⁰, 10⁻⁵, 10⁻¹, 1, 5, 10, 50, 10², 10³, 10⁵ or 10¹⁰%.

In some other cases, S_{I}/S_{A} or (S_{A}−S_{I})/S_{A} can be lower than 10⁵⁰, 10²⁰, 10¹⁰, 10⁵, 10, 5, 2, 1, 10⁻³, 10⁻⁵, 10⁻¹⁰ or 10⁻⁵⁰%.

In still some other cases, S_{I}/S_{A} or (S_{A}−S_{I})/S_{A} can be between 10⁻⁵⁰ and 10⁵⁰%, between 10⁻³ and 10³%, between 10⁻³ and 99.99%, between 10⁻² and 99%, between 10⁻¹ and 90%, between 1 and 85%, or between 5 and 60%.

In one embodiment of the invention, the alteration is or results in or is associated with an increase in particle size from the size of the initial particle smaller than 10⁵, 10³, 100, 50, 20, 10, 5, 3, 2 or 1 nm up to a size of the altered nanoparticle larger than 10⁻³, 0.1, 1, 5, 10 or 100 nm.

In another embodiment of the invention, the alteration is or results in or is associated with an increase in particle size from the size of the initial particle to the size of the altered particle by a quantity S_{A}−S_{I}, which is larger than 10⁻⁵, 10⁻¹, 1, 5, 10 or 10³ nm.

In another embodiment of the invention, the alteration is or results in or is associated with an increase in particle size from the size of the initial particle to the size of the altered particle by a quantity S_{A}−S_{I}, which is smaller than 10⁵, 10, 5, 1 or 10⁻¹ nm.

In one embodiment of the invention, the alteration is or results in or is associated with a decrease in FWHM (full width half maximum) or width W of the particle size distribution, preferentially from the FWHM or width W of the size distribution of the initial particle down to the FWHM or width W of the size distribution of the altered particle, where this decrease is such that FWHM_{A}/FWHM_{I}, (FWHM_{I}−FWHM_{A})/FWHM_{I}, W_{A}/W_{I}, or (W_{I}−W_{A})/W_{I} is between 10⁻³% and 99.99%, where FWHM_{A} and FWHM_{I} are the full width half maximum of the size distribution of the altered and initial particles, respectively, where W_{A} and W_{I} are the width of the size distribution of the altered and initial particles, respectively.

In some cases, FWHM_{A}/FWHM_{I}, (FWHM_{I}−FWHM_{A})/FWHM_{I}, W_{A}/W_{I} or (W_{I}−W_{A})/W_{I} can be larger than 10⁻⁵⁰, 10⁻²⁰, 10⁻¹⁰, 10⁻⁵, 10⁻¹, 1, 5, 10, 50, 10², 10³, 10⁵ or 10¹⁰%.

In some other cases, FWHM_{A}/FWHM_{I}, (FWHM_{I}−FWHM_{A})/FWHM_{I}, W_{A}/W_{I} or (W_{I}−W_{A})/W_{I} can be lower than 10⁵⁰, 10²⁰, 10¹⁰, 10⁵, 10, 5, 2, 1, 10⁻³, 10⁻⁵, 10⁻¹⁰ or 10⁻⁵⁰%.

In still some other cases, FWHM_{A}/FWHM_{I}, (FWHM_{I}−FWHM_{A})/FWHM_{I}, W_{A}/W_{I} or (W_{I}−W_{A})/W_{I} can be between 10⁻⁵⁰ and 10⁵⁰%, between 10⁻³ and 10³%, between 10⁻³ and 99.99%, between 10⁻² and 99%, between 10⁻¹ and 90%, between 1 and 85%, or between 5 and 60%.

In one embodiment of the invention, the alteration is or results in or is associated with a decrease in FWHM or width W of the particle size from a FWHM or W of the size distribution of the initial particle larger than 10⁻³, 0.1, 1, 5, 10 or 100 nm, down to a FWHM or W of the size distribution of the altered particle smaller than 10⁵, 10³, 100, 50, 20, 10, 5, 3, 2 or 1 nm.

In another embodiment of the invention, the alteration is or results in or is associated with a decrease in FWHM or W of the particle size distribution from the FWHM or W of size distribution of the initial particle to the FWHM or W of the size distribution of the altered particle by a quantity FWHM₁-FWHM_{A} or W_{I}−W_{A}, which is larger than 10⁻⁵, 10⁻¹, 1, 5, 10 or 10³ nm.

In another embodiment of the invention, the alteration is or results in or is associated with a decrease in FWHM or W of the particle size distribution from the FWHM or W of the size distribution of the initial particle to the FWHM or W of the size distribution of the altered particle by a quantity FWHM₁-FWHM_{A} or W_{I}−W_{A}, which is smaller than 10⁵, 10, 5, 1 or 10⁻¹ nm.

In one embodiment of the invention, the alteration is or results in or is associated with an increase in FWHM or width W of the particle size distribution, preferentially from the FWHM or width W of the size distribution of the initial particle up to the FWHM or width W of the size distribution of the altered particle, where this increase is such that FWHM_{I}/FWHM_{A}, (FWHM_{A}−FWHM_{I})/FWHM_{A}, W_{I}/W_{A}, or (W_{A}−W_{I})/W_{A} is between 10⁻³% and 99.99%, where FWHM_{A} and FWHM_{I} are the full width half maximum of the size distribution of the altered and initial particles, respectively, where W_{A} and W_{I} are the width of the size distribution of the altered and initial particles, respectively.

In some cases, FWHM_{I}/FWHM_{A}, (FWHM_{A}−FWHM_{I})/FWHM_{A}, W_{I}/W_{A} or (W_{A}−W_{I})/W_{A} can be larger than 10⁻⁵, 10⁻²⁰, 10⁻¹⁰, 10⁻⁵, 10⁻¹, 1, 5, 10, 50, 10², 10³, 10⁵ or 10¹⁰%.

In some other cases, FWHM_{I}/FWHM_{A}, (FWHM_{A}−FWHM_{I})/FWHM_{A}, W_{I}/W_{A} or (W_{A}−W_{I})/W_{A} can be lower than 10⁵⁰, 10²⁰, 10¹⁰, 10⁵, 10, 5, 2, 1, 10⁻³, 10⁻⁵, 10⁻¹⁰ or 10⁻⁵⁰%.

In still some other cases, FWHM_{I}/FWHM_{A}, (FWHM_{A}−FWHM_{I})/FWHM_{A}, W_{I}/W_{A} or (W_{A}−W_{I})/W_{A} can be between 10⁻⁵⁰ and 10⁵⁰%, between 10⁻³ and 10³%, between 10⁻³ and 99.99%, between 10⁻² and 99%, between 10⁻¹ and 90%, between 1 and 85%, or between 5 and 60%.

In one embodiment of the invention, the alteration is or results in or is associated with an increase in FWHM or width W of the particle size distribution from a FWHM or W of the size distribution of the initial particle smaller than 10⁵, 10³, 100, 50, 20, 10, 5, 3, 2 or 1 nm, up to a FWHM or W of the size distribution of the altered particle larger than 10⁻³, 0.1, 1, 5, 10 or 100 nm.

In another embodiment of the invention, the alteration is or results in or is associated with an increase in FWHM or W of the particle size distribution from the FWHM or W of size distribution of the initial particle up to the FWHM or W of the size distribution of the altered particle by a quantity FWHM_{A}-FWHM₁ or W_{A}−W_{I}, which is larger than 10⁻⁵, 10⁻¹, 1, 5, 10 or 10³ nm.

In another embodiment of the invention, the alteration is or results in or is associated with an increase in FWHM or W of the particle size distribution from the FWHM or W of the size distribution of the initial particle to the FWHM or W of the altered particle by a quantity FWHM_{A}−FWHM_{I}, or W_{A}−W_{I}, which is smaller than 10⁵, 10, 5, 1 or 10⁻¹ nm.

In one embodiment of the invention, the alteration is or results in or is associated with an increase in the number of peaks in the particle size distribution, where the number of peaks in the size distribution of the altered particle is larger than the number of peaks in the size distribution of the initial particle, preferentially by at least 1, 2, 5, 10, 10³ or 10⁵ peak(s). In some cases, the alteration can be an increase in the number of modes within the particle size distribution, preferentially from n modes in the size distribution of the initial particle up to m modes in the size distribution of the altered particle, where n, m are preferentially integers larger than 1, and m is an integer smaller than n.

In another embodiment of the invention, the alteration is or results in or is associated with a decrease in the number of peaks in the particle size distribution, where the number of peaks in the size distribution of the altered particle is smaller than the number of peaks in the size distribution of the initial particle, preferentially by at least 1, 2, 5, 10, 10³ or 10⁵ peak(s). In some cases, the alteration can be a decrease in the number of modes within the particle size distribution, preferentially from o modes in the size distribution of the initial particle down to p modes in the size distribution of the altered particle, where o, p are preferentially integers larger than 1, and o is an integer larger than p.

As an example of decrease in particle size following alteration, table 6 shows that an initial particle before alteration has a bi-modal size distribution with a dominant peak centered at 37.5 nm, and an altered particle after alteration by HCl treatment has a bi-modal size distribution with a dominant peak at 11 nm.

As an example of increase in particle size following alteration, table 6 shows that an initial particle before alteration has a bi-modal size distribution with a dominant peak centered d at 37.5 nm, and an altered particle following particle administration to mouse tumors has a dominant peak at 43 nm.

As an example of change in the number of peaks within the particle size distribution following alteration, table 6 shows that an initial particle before alteration has a bimodal size distribution, and an altered particle following particle administration to mouse tumor has a mono-modal size distribution. As an example of decrease of FWHM of the particle size distribution following alteration, table 6 shows that the FWHM decreases from 20 nm (P1, initial particle) down to 9 nm (P1, altered particle by being brought into contact with U87-Luc cells), from 35 nm (Pt, initial particle) down to 26.5 nm (Pt, altered particle by being brought into contact with U87-Luc cells), 35 nm (initial particle) to 20 nm (altered particle, particle mixed with HCl), 35 nm (initial particle) to 30 nm (altered particle administered to mouse tumor), 35 nm (initial particle) to 17.5 nm (altered particle administered to tumors and exposed to AMF). In these cases, the FHHM decrease by a factor of 1.3 to 2.2 following alteration.

As an example of increase of FWHM of the particle size distribution following alteration, table 6 shows that the FWHM increases from 15 nm (P2, initial particle) to 17.5 nm (P2, particle altered by being brought into contact with U87-Luc cells). In this case, the FWHM increase by a factor of 1.2.

In some cases, the variation (increase or decrease) in FWHM, size, number of peaks of the particle or particle size distribution can be larger than that/those reported in the examples, preferentially by a factor of more than 1.001, 1.1, 1.5, 2, 5, 10, 10², 10³ or 10⁵, preferentially when the conditions of alteration more strongly affect the properties of the particle, such as a lower pH used for alteration.

In some other cases, the variation (decrease or increase) in FWHM, size, number of peaks of the particle or particle size distribution can be smaller, preferentially by a factor of more than 1.001, 1.1, 1.5, 2, 5, 10, 10², 10³ or 10⁵, preferentially when the conditions of degradation are less strongly affecting the properties of the particle, such as a pH closer to 7 used for alteration.

In some cases, the number or concentration of initial compounds can exist or be measured or be estimated before or without alteration.

In some other cases, the number or concentration of altered compounds can exist or be measured or be estimated during or after alteration.

In one embodiment of the invention, the alteration is or results in or is associated with a decrease in the number or concentration of compounds bond to the nanoparticle, from a number nᵢ of initial compounds bond to the initial nanoparticle down to a number nₐ of altered compounds bond to the altered nanoparticle, where nᵢ/nₐ is preferentially between 1 and 10¹⁰.

In one embodiment of the invention, nᵢ, nₐ, or nᵢ−nₐ is larger than 10⁻⁵⁰, 10⁻²⁰, 10⁻¹⁰, 10⁻⁵, 10⁻², 10⁻¹, 1, 5, 10, 10³ or 10⁵ compounds or mg of compounds or compounds per particle or mg of compounds per mg of particle. In some other cases, nᵢ/nₐ is larger than 1, 2, 5, 10, 10³, 10⁵, 10¹⁰ or 10⁵⁰. This can occur when the alteration results in a large decrease of the number of compounds bond to the nanoparticle.

In some other cases, nᵢ, nₐ, nᵢ−nₐ can be smaller than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ compounds or mg of compounds or compounds per particle or mg of compounds per mg of particle. In some other cases, nᵢ/nₐ is smaller than 10⁵⁰, 10¹⁰, 10⁵, 10³, 10, 5, 2 or 1. This can occur when the alteration results in a small decrease of the number of compounds bond to the nanoparticle.

In still some other cases, nᵢ/nₐ can be between 10⁻⁵⁰ and 10⁵⁰, 10⁻¹⁰ and 10²⁰, 10⁻⁵ and 10²⁰, 10⁻³ and 10²⁰, 10⁻¹ and 10¹⁰, or between 1 and 10¹⁰.

In one embodiment of the invention, the alteration is or results in or is associated with a decrease of the number or concentration of compounds bound to the nanoparticle from a number nᵢ larger than 10⁻⁵⁰, 10⁻²⁰, 10⁻¹⁰, 10⁻⁵, 10⁻², 10⁻¹, 1, 5, 10, 10³ or 10⁵ compounds or mg of compounds or compounds per nanoparticle or mg of compounds per mg of nanoparticles down to a number nₐ lower than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ compounds or mg of compounds or compounds per nanoparticle or mg of compounds per mg of nanoparticles.

As an example, table 2 shows that the concentration of endotoxins, which can be the compound, in a suspension comprising 40 μg of magnetosomes decreases from 1.6 EU before alteration down to 8.9 10⁻² EU after alteration with HCl, resulting in a decrease by a factor of 18 of the concentration of endotoxins between before and after alteration. In some cases, the alteration can yield a decrease in the number or concentration of the compound bond to the nanoparticle by a larger factor than 18, preferentially a factor at least equal to 20, 50, 10², 10³, 10⁵ or 10¹⁰. This can be the case when the altering conditions are stronger and/or enable to remove or detach or release a larger quantity of compounds from the nanoparticles than those using the HCl treatment. In some other cases, the alteration can yield a decrease in the concentration of the compound bond to the nanoparticle by a lower factor than 18, preferentially a factor lower than 20, 10, 5, 2, 1, 10⁻³, 10⁻¹⁰, 10⁻¹⁰⁰. This can be the case when the altering conditions are softer and/or enable to remove or detach or release a lower quantity of compounds from the nanoparticles than those using the HCl treatment.

In some cases, the decrease in number or concentration of compounds bound to the nanoparticle can be or be associated with an increase in the number or concentration of compounds released from the nanoparticle.

In one embodiment of the invention, the alteration is or results in or is associated with the increase in the number or concentration of compounds released from the nanoparticles. The number or concentration of compounds released from the nanoparticle can increase from a number n_{RI} before alteration, where n_{RI} is preferentially the number of initial compounds released from the initial nanoparticle, up to a number n_{RA}, where n_{RA} is preferentially the number of altered compounds released from the altered nanoparticle.

In some cases, n_{RI} can be smaller than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ compounds or mg of compounds or compounds per nanoparticle or mg of compounds per mg of nanoparticles. Most preferentially, n_{RI} is equal to 0.

In some other cases, n_{RA} can be larger than 10⁻⁵⁰, 10⁻²⁰, 10⁻¹⁰, 10⁻⁵, 10⁻², 10⁻¹, 1, 5, 10, 10³ or 10⁵ compounds or mg of compounds or compounds per nanoparticle or mg of compounds per mg of nanoparticles.

In still some other cases, abs(n_{RA}−n_{RI}), n_{RA}/n_{RI}, n_{RI}/n_{RA}, abs(n_{RA}−n_{RI})/n_{RA} or abs(n_{RA}−n_{RI})/n_{RI} is larger than 10⁻⁵⁰, 10⁻²⁰, 10⁻¹⁰, 10⁻⁵, 10⁻¹, 1, 5, 10, 10³, 10⁵, 10¹⁰ or 10⁵⁰. Abs can designate the absolute value.

In still some other cases, abs(n_{RA}−n_{RI}), n_{RA}/n_{RI}, n_{RI}/n_{RA}, abs(n_{RA}−n_{RI})/n_{RA} or abs(n_{RA}−n_{RI})/n_{RI} is lower than 10⁵⁰, 10²⁰, 10¹⁰, 10⁵, 10³, 10, 5, 1, 10⁻¹, 10⁻⁵, 10⁻¹⁰ or 10⁻⁵.

In one embodiment of the invention, the alteration results in a percentage of compounds released from the nanoparticles larger than 10⁻⁵⁰, 10⁻²⁰, 10⁻¹⁰, 10⁻⁵, 10⁻¹, 1, 5, 10, 10³, 10⁵, 10¹⁰ or 10⁵⁰%. This percentage can be equal to N_{R}/(N_{R}+N_{NR}), where N_{R} and N_{NR} are the concentration or number of compounds released from the nanoparticles, and the concentration or number of compounds not released from the nanoparticles or bound to the nanoparticle, respectively.

In some cases, the percentage of compounds released from the nanoparticles is larger after, during or with alteration than before or without alteration, preferentially by a factor of at least 1.001, 1.1, 1.2, 1.5, 2, 5, 10, 10³, 10⁵ or 10¹⁰.

In some cases, the strength of at least one bond between the compound and the nanoparticle can be the binding strength or the strength with which the compound is bound to the nanoparticle.

In one embodiment of the invention, the alteration is or results in or is associated with a decrease or increase of the strength of at least one bond between the compound and the nanoparticle, preferentially from a strength Sᵢ of at least one initial bond between the initial compound and the initial nanoparticle to a strength Sₐ of at least one altered bond between the altered compound and the altered nanoparticle.

In some cases, Sᵢ and Sₐ can be the strengths of at least one bond that link(s) at least one compound to at least one nanoparticle.

In some other cases, Sᵢ and Sₐ can be the energies of at least one bond that link(s) at least one compound to at least one nanoparticle.

In some cases, Sₐ can be lower than Sᵢ if: i) at least one altered compound is linked to at least one altered nanoparticles via a number of bonds that is lower than the number of bonds that link at least one initial compound to at least one initial nanoparticle ii) at least one initial compound is linked to at least one initial nanoparticles via strong bonds and/or iii) at least one altered compound is linked to at least one altered nanoparticle via weak bonds.

In some cases, weak bonds can be Van der Waals interactions, dipole-dipole interactions, London dispersion force, and/or hydrogen bonding.

In some other cases, strong bonds can be covalent, ionic, and/or metallic bonds.

In some cases, Sᵢ and/or Sₐ can be larger than 10⁻⁵⁰, 10⁻²⁰, 10⁻¹⁰, 10⁻⁵, 10⁻¹, 1, 5, 10, 10³, 10⁵, 10¹⁰, 10²⁰, 10⁵⁰ or 10¹⁰⁰ eV, KJ, or Kcal, preferentially as measured per: i) mol of particle, nanoparticle, compound, or bond, or ii) particle, nanoparticle, compound, or bond.

In some other cases, Sᵢ and/or Sₐ can be lower than 10⁵⁰, 10²⁰, 10¹⁰, 10⁵, 10, 5, 2, 1, 10⁻¹, 10⁻³, 10⁻⁵, 10⁻¹⁰ or 10⁻²⁰ eV, KJ, or Kcal, preferentially as measured per: i) mol of particle, nanoparticle, compound, or bond, or ii) particle, nanoparticle, compound, or bond.

In still some other cases, Sᵢ/Sₐ can be larger than 10⁻⁵⁰, 10⁻²⁰, 10⁻¹⁰, 10⁻⁵, 10⁻¹, 1, 5, 10, 20, 50, 10², 10³, 10⁵ or 10¹⁰.

In one embodiment of the invention, the alteration is or results in or is associated with the breaking of at least one bond between the altered compound and the altered nanoparticle. In some cases, the alteration can be the release of at least one compound from at least one nanoparticle.

In one embodiment of the invention, the alteration is or results in or is associated with the weakening of at least one bond between the altered compound and the altered nanoparticle.

In some cases, the weakening of the bond between the compound and the nanoparticle can be a decrease of the bond forces, bond energies, interaction forces, or interaction energies between the compound and the nanoparticle.

In some cases, the breaking of the bond between the compound and the nanoparticle can be or be due to the removal or annihilation or decrease of the bond forces, bond energies, interaction forces, or interaction energies between the compound and the nanoparticle.

In some cases, the weakening of the bond between the compound and the nanoparticle can be a decrease of the dissociation energy of the bond between the compound and the nanoparticle.

In some cases, the breaking of the bond between the compound and the nanoparticle can be or be due to the removal or annihilation or decrease of the dissociation energy of the bond between the compound and the nanoparticle.

In some cases, the bond forces, bond energies, interaction forces, or interaction energies between the compound and the nanoparticle can be equal, proportional to, or related to the dissociation energy of the bond.

In some cases, the larger or the stronger the dissociation energy of the bond, the larger or the stronger the bond forces, bond energies, interaction forces, or interaction energies between said nanoparticles and said compound.

In some other cases, the lower or the weaker the dissociation energy of the bond, the lower or the weaker the bond forces, bond energies, interaction forces, or interaction energies between said nanoparticles and said compound.

In still some other cases, the dissociation energy of the bond can be the energy that needs to be provided or brought to or absorbed by or received by or transferred to the bond, nanoparticle, and/or particle, preferentially an energy due to or originating from or provided by the radiation and/or physico-chemical disturbance, to dissociate the compound and/or bond from the nanoparticle.

In one embodiment of the invention, the types of bonds that are weakened or broken by alteration are strong bonds.

In another embodiment of the invention, the types of bonds that are not weakened or not broken by alteration are strong bonds.

In one embodiment of the invention, the types of bonds that are weakened or broken by alteration are weak bonds.

In one embodiment of the invention, the types of bonds that are not weakened or not broken by alteration are weak bonds.

In another embodiment of the invention, the number of bonds that is broken or weakened by alteration is larger than 1, 5, 10, 10³, 10⁵ or 10¹⁰ bonds per particle or nanoparticle or per mg of particle or nanoparticle or per cm³ of body part or altering medium.

In another embodiment of the invention, the number of bonds that is broken or weakened by alteration is smaller than 10¹⁰, 10⁵, 10, 5, 3 or 1 bonds per particle or nanoparticle or per mg of particle or nanoparticle or per cm³ of body part or altering medium.

In one embodiment of the invention, the alteration is or results in or is associated with a decrease or increase of the bond-dissociation energy between the compound and the nanoparticle, preferentially from a bond-dissociation energy E_{di} between the initial compound and the initial nanoparticle down to or up to a bond-dissociation energy E_{da} between the altered compound and the altered nanoparticle.

In some cases, E_{di} and/or E_{da} can be larger than 10⁻⁵⁰, 10⁻²⁰, 10⁻¹⁰, 10⁻⁵, 10⁻¹, 1, 5, 10, 10³, 10⁵, 10¹⁰, 10²⁰, 10⁵⁰ or 10¹⁰⁰ eV, KJ, or Kcal, preferentially as measured per: i) mol of particle, nanoparticle, compound, or bond, or ii) particle, nanoparticle, compound, or bond.

In some other cases, E_{di} and/or E_{da} can be lower than 10⁵⁰, 10²⁰, 10¹⁰, 10⁵, 10, 5, 1, 10⁻¹, 10⁻³, 10⁻⁵, 10⁻¹⁰ or 10⁻ eV, KJ, or Kcal, preferentially as measured per: i) mol of particle, nanoparticle, compound, or bond, or ii) particle, nanoparticle, compound, or bond.

In still some other cases, E_{di}/E_{da} can be lower than 10⁵⁰, 10¹⁰, 10⁵, 10, 5, 2, 1, 10⁻⁵, 10⁻¹⁰ or 10⁻⁵⁰. This can be the case when the compound is more strongly bound after alteration than before alteration.

In still come other cases, E_{di}/E_{da} can be larger than 10⁻⁵⁰, 10⁻¹⁰, 10⁻⁵, 10⁻¹, 1, 5, 10, 10⁵, 10¹⁰ or 10⁵⁰.

This can be the case when the compound is less strongly bound after alteration than before alteration.

In one embodiment of the invention, the alteration is or results in or is associated with an increase of the bond-dissociation energy between the compound and the nanoparticle, preferentially from a bond-dissociation energy E_{di} between the initial compound and the initial nanoparticle up to a bond-dissociation energy E_{da} between the altered compound and the altered nanoparticle.

In some cases, the coating of the nanoparticle consists of a material that surrounds or envelops or embeds the nanoparticle.

In some cases, the coating of the nanoparticle can comprise the compound as defined in the invention.

In some other cases, the compound can be bound or attached to the coating of the nanoparticle.

In still another embodiment of the invention, the alteration is or results in or is associated with a decrease or increase of the thickness of the coating of said nanoparticle.

In some cases, the coating thickness is not uniform or the coating only partly surrounds the nanoparticles.

In some other cases, the coating thickness is uniform or the coating fully surrounds the nanoparticles.

In some cases, the coating thickness can be the thickness of the coating measured at least one site of the nanoparticle(s).

In some cases, the coating thickness can be the average thickness of the coatings of the nanoparticle(s).

In some cases, the coating thickness can be larger than 10⁻¹, 1, 5, 10, 10³ or 10⁵ nm, preferentially in the initial nanoparticle, preferentially before or without alteration.

In some other cases, the coating thickness can be smaller than 10⁵, 10³, 10, 5, 1 or 10⁻¹ nm, preferentially in the altered nanoparticle, preferentially during, after or with alteration.

In still some other cases, the coating thickness decreases, preferentially by a factor of at least 1.001, 1.1, 1.5, 2, 5, 10, 10³ or 10⁵ between before and after alteration.

In one embodiment of the invention, the alteration is or results in or is associated with a decrease of the coating thickness of the nanoparticle, from a coating thickness CTᵢ of the initial nanoparticle down to a coating thickness CTₐ of the altered nanoparticle.

In some cases, the coating thickness can decrease from CTᵢ larger than 10⁻³, 0.1, 1, 5, 10 or 100 nm down to CTₐ smaller than 10⁵, 10³, 100, 50, 20, 10, 5, 3, 2 or 1 nm.

In some other cases, the coating thickness can decrease between before and after alteration by a quantity of at least 10⁻⁵, 10⁻¹, 1, 5, 10 or 10³ nm.

In some cases, CTᵢ can be larger than 10⁻¹⁰, 10⁻⁵, 10⁻¹, 1, 5, 10, 10³ or 10⁵ nm or can be larger than α·Sᵢ, where α is a proportionality coefficient preferentially larger than 10⁻¹⁰, 10⁻⁵, 10⁻¹, 1, 5, 10, 10³ or 10⁵, and Sᵢ is the size of the initial nanoparticle preferentially larger than 10⁻¹⁰, 10⁻⁵, 10⁻¹, 1, 5, 10, 10³ or 10⁵ nm.

In some other cases, CTₐ can be larger than 10⁻¹⁰, 10⁻⁵, 10⁻¹, 1, 5, 10, 10³ or 10⁵ nm or can be larger than α·Sₐ, where α is a proportionality coefficient preferentially larger than 10⁻¹⁰, 10⁻⁵, 10⁻¹, 1, 5, 10, 10³ or 10⁵, and Sₐ is the size of the altered nanoparticle preferentially larger than 10⁻¹⁰, 10⁻⁵, 10⁻¹, 1, 5, 10, 10³ or 10⁵ nm.

In some cases, CTᵢ can be smaller than 10⁵⁰, 10¹⁰, 10⁵, 10, 5, 2, 1, 10⁻¹, 10⁻³ or 10⁻⁵ nm or can be smaller than α·Sᵢ, where α is a proportionality coefficient preferentially larger than 10⁻¹⁰, 10⁻⁵, 10⁻¹, 1, 5, 10, 10³ or 10⁵, and Sᵢ is the size of the initial nanoparticle preferentially smaller than 10⁵⁰, 10¹⁰, 10⁵, 10, 5, 2, 1, 10⁻¹, 10⁻³ or 10⁻⁵ nm.

In some other cases, CTₐ can be smaller than 10⁵⁰, 10¹⁰, 10⁵, 10, 5, 2, 1, 10⁻¹, 10⁻³ or 10⁻⁵ nm or can be larger than α·Sₐ, where α is a proportionality coefficient preferentially larger than 10⁻¹⁰, 10⁻⁵, 10⁻¹, 1, 5, 10, 10³ or 10⁵, and Sₐ is the size of the altered nanoparticle preferentially smaller than 10⁵⁰, 10¹⁰, 10⁵, 10, 5, 2, 1, 10⁻¹, 10⁻³ or 10⁻⁵ nm.

In still some other cases, CTᵢ/CTₐ is larger than 10⁻⁵⁰, 10⁻²⁰, 10⁻¹⁰, 10⁻⁵, 10⁻¹, 1, 5, 10, 10³, 10⁵, 10¹⁰ or 10²⁰.

In still some other cases, CTᵢ/CTₐ is smaller than 10⁵⁰, 10¹⁰, 10⁵, 1, 10⁻⁵, 10⁻¹⁰ or 10⁻⁵.

In still some other cases, CTᵢ/CTₐ is between 10⁻⁵⁰ and 10⁵⁰, between 10⁻¹⁰ and 10¹⁰, between 10⁻⁵ and 10⁵, between 10⁻³ and 10³, between 10⁻¹ and 10, or between 0.2 and 5.

As an example, table 6 shows that the size of the nanoparticle can decrease between before and after alteration, which can be due to a decrease in coating thickness.

In one embodiment of the invention, the alteration is or results in or is associated with an increase of the coating thickness of the nanoparticle, from a coating thickness CTᵢ of the initial nanoparticle up to a coating thickness CTₐ of the altered nanoparticle.

In some cases, the coating thickness can increase from a value of CTᵢ smaller than 10⁵, 10³, 100, 50, 20, 10, 5, 3, 2 or 1 nm, to a value of CTₐ larger than 10⁻³, 0.1, 1, 5, 10 or 100 nm.

In still some other cases, the coating thickness can increase by a quantity of at least 10⁻⁵, 10⁻¹, 1, 5, 10 or 10³ nm between before and after alteration.

As an example, table 6 shows that the size of the nanoparticle can increase between before and after alteration, which can be due to an increase in coating thickness.

In one embodiment of the invention, the alteration is or results in or is associated with a decrease or increase of the cluttering of the compound bound to the nanoparticle, preferentially a decrease from a large cluttering of the initial compound bound to the initial nanoparticle down to a low cluttering of the altered compound bound to the altered nanoparticle. In some cases, a large cluttering of the initial compound bound to the initial nanoparticle can represent a large level of cluttering of these compounds, which can be due to the large number or concentration of these compounds, preferentially located in the coating or at the surface of the nanoparticle, where the cluttering of these compounds can be considered as large relatively to the cluttering of the altered compounds. In some other cases, a low cluttering of the altered compound bound to the altered nanoparticle can represent a low level of cluttering of these compounds, which can be due to the small number or concentration of these compounds, preferentially located in the coating or at the surface of the nanoparticle, where the cluttering of these compounds can be considered as low relatively to the cluttering of the initial compounds.

In still another embodiment of the invention, the alteration is a decrease of the cluttering of the compounds bound to the nanoparticle. In some cases, the decrease of the cluttering of the compound bound to the nanoparticle can be or correspond to or result in or lead to or be associated with: i) a decrease in the number or concentration of compound bound to the nanoparticle that imped or block the release of the compound from the nanoparticle, or ii) an increased faculty of the nanoparticle to release the compound from the nanoparticle due to a lower number of compounds bound to the nanoparticle that block or imped such release.

In one embodiment of the invention, the alteration is or results in or is associated with an increase of the cluttering of the compound bound to the nanoparticle, from a small cluttering of the initial compound bound to the initial nanoparticle up to a large cluttering of the altered compound bound to the altered nanoparticle.

In one embodiment of the invention, the alteration is or results in or is associated with a decrease of the number or concentration of compounds N₁ that prevent the release of compounds N₂ from the nanoparticle, from a number of initial compounds N₁ᵢ that prevent the release of initial compounds N₂ᵢ from the initial nanoparticle down to a number of altered compounds N₁ₐ that prevent the release of altered compounds N₂ₐ from the altered nanoparticle.

In some cases, the sum of N₁ᵢ+N₂ᵢ is the total number or concentration of compounds bound to the initial nanoparticle.

In some cases, the sum N₁ₐ+N₂ₐ is the total number or concentration of compounds bound to the altered nanoparticle.

In some cases, N₁ᵢ and/or N₁ₐ can be smaller than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ compounds or mg of compounds or compounds per nanoparticle or mg of compounds per mg of nanoparticles.

In some other cases, N₁ᵢ and/or N₁ₐ can be larger than 10⁻⁵⁰, 10⁻²⁰, 10⁻¹⁰, 10⁻⁵, 10⁻², 10⁻¹, 1, 5, 10, 10³ or 10⁵ compounds or mg of compounds or compounds per nanoparticle or mg of compounds per mg of nanoparticles.

In still some other cases, abs(N₁ₐ−N₁ᵢ)/N₁ᵢ, N₁ᵢ/N₁ₐ, abs(N₁ₐ−N₁ᵢ)/N₁ₐ or abs(N₁ₐ−N₁ᵢ)/N₁ᵢ is larger than 10⁻⁵, 10⁻²⁰, 10⁻¹⁰, 10⁻⁵, 10⁻¹, 1, 5, 10, 10³, 10⁵, 10¹⁰ or 10⁵⁰.

In still some other cases, abs(N₁ₐ−N₁ᵢ)/N₁ᵢ, N₁ᵢ/N₁ₐ, abs(N₁ₐ−N₁ᵢ)/N₁ₐ or abs(N₁ₐ−N₁ᵢ)/N₁ᵢ is lower than 10⁵⁰, 10²⁰, 10¹⁰, 10⁵, 10³, 10, 5, 1, 10⁻¹, 10⁻⁵, 10⁻¹⁰ or 10⁻⁵⁰.

In one embodiment of the invention, the alteration is or results in or is associated with a modification of the chemical composition of the particle, preferentially of the initial particle, also designated as chemical modification or first chemical modification.

In some cases, the chemical modification can be the change of more than 1, 2, 5, 10, 10³, 10⁵, 10¹⁰, 10²⁰, 10⁵⁰ or 10¹⁰⁰ chemical element(s) comprised in the particle, between before and after alteration.

In some other cases, the chemical modification can be the change of less than 10¹⁰⁰, 10⁵⁰, 10²⁰, 10¹⁰, 10⁵, 10³, 10, 5, 2 or 1 chemical element(s) comprised in the particle, between before and after alteration.

In some other cases, the chemical modification can be the change of more than 10⁻⁵⁰, 10⁻¹⁰, 10⁻¹, 1, 5, 10, 20, 50, 75, 80, 90 or 99%, preferentially by mass or volume, of the chemical elements comprised in the particle between before after alteration. This percentage can be the ratio between the number or concentration or mass of chemical elements comprised in the altered particle divided by the number or concentration or mass of chemical elements comprised in the initial particle.

In some other cases, the chemical modification can be the change of less than 100, 99, 90, 70, 60, 50, 20, 10, 5, 2, 1 or 10⁻³% preferentially by mass or volume, of the chemical elements comprised in the particle between before after alteration.

In still some other cases, the chemical modification can be the replacement of at least one chemical element by another chemical element in the particle or the loss or release of at least one chemical element by the particle or the gain of at least one chemical element by the nanoparticles, preferentially between before and after alteration.

In some cases, a chemical element can be an atom or an ion.

In some cases, the chemical modification can be a change from a metallic to a non-metallic composition of the particle, between before and after alteration.

In some cases, the chemical modification can be a change from a more metallic composition before alteration to a less metallic composition after alteration.

In some other cases, the chemical modification can be change from a composition comprising more than 1, 5, 10, 10³, 10⁵, 10¹⁰, 10⁵⁰ or 10¹⁰⁰ metallic atom(s), preferentially per particle, before alteration, to a composition comprising less than 10¹⁰⁰, 10⁵⁰, 10¹⁰, 10⁵, 10³, 10, 5 or 1 metallic atom(s), preferentially per particle, after alteration.

In some other cases, the chemical modification can be a change from a composition comprising more than 10⁻⁵⁰, 10⁻¹⁰, 1, 5, 10, 50, 75, 90 or 99% of metallic atom(s), preferentially by mass, number or volume, preferentially per particle, before alteration, to a composition comprising less than 99, 90, 75, 50, 10, 5, 1 or 10⁻³% of metallic atom(s), preferentially by mass, number, or volume, preferentially per particle, after alteration. This percentage may be the ratio between the number, concentration, mass or volume of metallic atom(s) comprised in the particle and the number, concentration, mass or volume of all atom(s) in the particle.

In some other cases, the chemical modification can be a change from a metallic to a non-metallic composition of the particle.

In some other cases, the chemical modification can be a change from a non-metallic to a metallic composition of the particle.

In some cases, a metallic composition can be a composition, preferentially of the particle, in which the particle comprise more than 10⁻⁵⁰, 10⁻¹⁰, 10⁻⁵, 1, 5, 10, 50, 70, 90 or 99%, preferentially by mass, number, or volume, of metallic atoms, preferentially per particle. This percentage may be the ratio between the number of metallic atoms in the particle and the total number of atoms in the particle.

In some other cases, a non-metallic composition can be a composition, in which the particle comprises less than 10⁵⁰, 10¹⁰, 10⁵, 10³, 10, 5, 2, 1, 10⁻² or 10⁻⁵%, preferentially by mass, number, or volume, of metallic atoms, preferentially per particle.

In one embodiment of the invention, the chemical modification is a change from a non-pyrogenic to a pyrogenic composition or from a pyrogenic to a non-pyrogenic composition. In some cases, a non-pyrogenic composition is a composition associated with a concentration in endotoxins or lipopolysaccharide that is lower than 10⁵⁰, 10¹⁰, 10⁵, 10, 1, 10⁻¹, 10⁻⁵ or 10⁻¹⁰ EU per mg or per mL or per mg per mL of particle or particle suspension or altering medium or body part. In some other cases, a non-pyrogenic composition is a composition that triggers an increase in temperature of the body part or individual of less than 10⁵, 10³, 100, 50, 25, 10, 5, 2, 1 or 10⁻⁵° C. (degree Celsius). In some cases, a pyrogenic composition is a composition that comprises a concentration in endotoxins or lipopolysaccharide that is larger than 10⁻⁵⁰, 10⁻¹⁰, 10⁻⁵, 10⁻¹, 1, 10, 10⁵ or 10¹⁰ EU per mg or per mL or per mg per mL of particle or particle suspension or degrading medium or body part. In some cases, a pyrogenic composition is a composition that triggers an increase in temperature of the body part or individual of more than 10⁻⁵, 10⁻³, 10⁻¹, 1, 5, 10 or 20° C. (degree Celsius).

In one embodiment of the invention, the chemical modification or alteration is the change from a non-immunogenic to an immunogenic composition of the particle or from an immunogenic to a non-immunogenic composition of the particle. In some cases, a non-immunogenic composition is a composition that triggers the appearance or migration or transport, preferentially in the body part, of a number of immune cells such as T cells, B cells, dendritic cells, antigen presenting cells, macrophages or other immune entities such as chemokines or interleukins or cytokines that is lower than 10⁵⁰, 10¹⁰, 10⁵, 10, 1, 10⁻¹, 10⁻⁵ or 10⁻¹⁰ preferentially per cm³ or mL of body part. In some other cases, an immunogenic composition is a composition that triggers the appearance or migration or transport, preferentially in the body part, of more than 0, 10⁻⁵⁰, 10⁻¹⁰, 10⁻⁵, 1, 10, 10⁵ or 10¹⁰ immune cell(s) such as T cells, B cells, dendritic cells, antigen presenting cells, macrophages or other immune entities such as chemokines or interleukins or cytokines, preferentially per cm³ or mL of body part.

In one embodiment of the invention, the chemical modification is the change from a non-pharmacological to a pharmacological composition or from a pharmacological to a non-pharmacological composition. In some cases, a non-pharmacological composition is a composition that comprises less than 10⁵⁰, 10¹⁰, 10⁵, 10, 1, 10⁻¹, 10⁻⁵ or 10⁻¹⁰ pharmacological compounds or mg of pharmacological compounds, preferentially per particle or per mg of particle. In some other cases, a pharmacological composition is a composition that comprises more 10⁻⁵⁰, 10⁻¹⁰, 10⁻⁵, 1, 1, 10, 10⁵ or 10¹⁰ pharmacological compounds or mg of pharmacological compounds, preferentially per particle or per mg of particle. In some cases, a pharmacological compound can have a pharmacological activity, preferentially when it is activated, preferentially after it is released from the nanoparticle, such as an activity against pathological or tumor cells. In some other cases, a pharmacological compound can be non-pharmacologically active, preferentially when it is bound to the nanoparticle.

In one embodiment of the invention, the chemical modification is the change from a non-metabolic to a metabolic composition or from a metabolic to a non-metabolic composition. In some cases, a non-metabolic composition is a composition that comprises less than 10⁵⁰, 10¹⁰, 10⁵, 10, 0, 1, 10⁻¹, 10⁻⁵ or 10⁻¹⁰ metabolic compounds or mg of metabolic compounds, preferentially per particle or per mg of particle. In some other cases, a metabolic composition is a composition that comprises more 10⁻⁵⁰, 10⁻¹⁰, 10⁻⁵, 0, 1, 10, 10⁵ or 10¹⁰ metabolic compounds or mg of metabolic compounds, preferentially per particle or per mg of particle. In some cases, a metabolic compound can have a metabolic activity, preferentially when it is activated, preferentially after it is released from the nanoparticle, such as an activity against pathological or tumor cells. In some other cases, a metabolic compound can be non-metabolically active, preferentially when it is bound to the nanoparticle.

In one embodiment of the invention, the chemical modification is the change from a non-immunogenic to an immunogenic composition or from an immunogenic to a non-immunogenic composition. In some cases, a non-immunogenic composition is a composition that comprises less than 10⁵⁰, 10¹⁰, 10⁵, 10, 1, 10⁻¹, 10⁻⁵ or 10⁻¹⁰ immunogenic compounds or mg of immunogenic compounds, preferentially per particle or per mg of particle. In some other cases, an immunogenic composition is a composition that comprises more 10⁻⁵⁰, 10⁻¹⁰, 10⁻⁵, 1, 1, 10, 10⁵ or 10¹⁰ immunogenic compounds or mg of immunogenic compounds, preferentially per particle or per mg of particle. In some cases, an immunogenic compound can have an immunogenic activity, preferentially when it is activated, preferentially after it is released from the nanoparticle, such as an activity against pathological or tumor cells. In some other cases, an immunogenic compound can be non-immunogenically active, preferentially when it is bound to the nanoparticle.

In one embodiment of the invention, the chemical modification is the activation of a vaccine, i.e. preferentially without the chemical modification the vaccine is not active.

In another embodiment of the invention, the alteration is or results in or is associated with a decrease of the surface charge or zeta potential of the particle, preferentially from a surface charge of the initial particle SCᵢ or zeta potential of the initial particle ZPᵢ down to a surface charge of the altered particle SCₐ or zeta potential of the altered particle ZPₐ.

In some cases, the property of the particle such as the surface charge or zeta potential of the particle is measured or exists before alteration or without alteration.

In some other cases, the property of the particle such as the surface charge or zeta potential of the particle is measured or exists during or after or with alteration.

In some cases, SCᵢ, ZPᵢ, SCₐ, and/or ZPₐ can be smaller than 10⁵⁰, 10¹⁰, 10⁵, 10³, 100, 50, 20, 10, 5, 2, 1, 0, −5, −10, −50 or −100 mV.

In some other cases, SCᵢ, ZPᵢ, SCₐ, and/or ZPₐ can be larger than −10¹⁰, −10⁵, −10³, −100, −50, −20, −10, −5, −1, 0, 2, 5, 10, 50, 10² or 10⁵ mV.

In still some other cases, SCᵢ, ZPᵢ, SCₐ, and/or ZPₐ can be between −10⁵⁰ mV and 10⁵⁰ mV, between −10¹⁰ mV and 10¹⁰ mV, between −10⁵ mV and 10⁵ mV, between −10³ mV and 10³ mV, between −100 mV and 100 mV, between −50 mV and 50 mV, or between −20 mV and 20 mV.

In still some other cases, SCᵢ/SCₐ or ZPᵢ/ZPₐ can be larger than 10⁻⁵⁰, 10⁻¹⁰, 10⁻⁵, 10⁻³, 0, 1, 2, 5, 10, 10³, 10⁵ or 10¹⁰.

In still some other cases, the zeta potential and/or surface charge can decrease from ZPᵢ or SCᵢ larger than −10⁵, −10³, −100, −50, −20, −10, −5, −2, −1, 0, 1, 2, 5, 10, 20 or 50 mV, preferentially before or without alteration to SCₐ or ZPₐ smaller than 10¹⁰, 10⁵, 10³, 10, 5, 2, 1, 0, −1, −5, −10, −50 or −100 mV, preferentially during, after or with alteration.

In still some other cases, the zeta potential and/or surface charge can decrease between before and after alteration by a magnitude or value larger than 10⁻²⁰, 10⁻⁵, 10⁻¹, 1, 5, 10, 20 or 100 mV.

In still some other cases, the zeta potential and/or surface charge can increase from ZPᵢ or SCᵢ smaller than 10⁵, 10³, 500, 100, 50, 20 or 10 mV up to SCₐ or ZPₐ larger than −10⁵, −10³, −10⁻¹, 0, 1, 5, 10, 50 or 100 mV.

In still some other cases, the zeta potential and/or surface charge can increase between before and after alteration by a magnitude or value larger than 10⁻²⁰, 10⁻¹, 10⁻¹, 1, 5, 10, 20 or 100 mV.

As an example, table 6 shows that the alteration can result in a variation of the surface charge of the nanoparticles.

In some case, the variation of the surface charge or zeta potential of the particle can depend on: i) the material used for the alteration, i.e. preferentially a degrading medium comprising positively charged ions may bring a more positive surface charge to the particle while a degrading medium comprising negatively charged ions may bring a more negative charge to the particle, or ii) the quantity of particle or coating removed by the degrading medium, i.e. for a large quantity of coating removed, the surface charge can become the surface charge of the core of the nanoparticles while for low quantity of coating removed the surface charge can remain the surface charge of the coating material or be close to this value.

In another embodiment of the invention, the alteration is or results in or is associated with a decrease of the isoelectric point of the particle. In some cases, this decrease can be a decrease from an isoelectric point of the initial particle of pH larger than 0, 1, 2, 3, 4, 5, 6, 7, 8, 9, 10, 11, 12, 13 or 14, preferentially before or without alteration, to an isoelectric point of the altered point of pH lower than 14, 13, 12, 11, 10, 9, 8, 7, 6, 5, 4, 3, 2 or 1, preferentially during, after or with alteration. In some other cases, this decrease is a decrease of the isoelectric point of the particle by a magnitude of at least 10⁻⁵⁰, 10⁻¹⁰, 10⁻⁵, 10⁻¹, 1, 2, 5 or 10 pH units, preferentially between before and after alteration.

In another embodiment of the invention, the alteration is or results in or is associated with an increase of the isoelectric point of the particle. In some cases, this increase can be an increase from an isoelectric point of the initial particle of pH smaller than 14, 13, 12, 11, 10, 9, 8, 7, 6, 5, 4, 3, 2 or 1, preferentially before or without alteration, to an isoelectric point of the altered point of pH larger than 0, 1, 2, 3, 4, 5, 6, 7, 8, 9, 10, 11, 12, 13 or 14, preferentially measured during, after, or with alteration.

In some other cases, this increase can be an increase of the particle isoelectric point alteration by a magnitude of at least 10⁻⁵⁰, 10⁻¹⁰, 10⁻⁵, 10⁻¹, 1, 2, 5 or 10 pH units.

In still another embodiment of the invention, the alteration is or results in or is associated with a modification, preferentially a decrease, of the percentage in mass of organic material or carbon or carbonaceous material of the particle.

In some cases, the percentage in mass of organic material or carbon or carbonaceous material of the particle can be larger than 10⁻¹⁰, 10⁻⁵⁰, 10⁻¹⁰, 10⁻⁵, 10⁻¹, 1, 5, 10, 50, 70 or 99%, preferentially before or without alteration.

In some other cases, the percentage in mass of organic material or carbon or carbonaceous material of the particle can be smaller than 100, 99, 70, 50, 10, 5, 2, 1, 10⁻¹ or 10⁻⁵%, preferentially during, after or with alteration.

In still some other cases, the percentage in mass of organic material or carbon or carbonaceous material of the particle can decrease, preferentially by a factor of at least 1.001, 1.1, 1.5, 2, 5, 10, 10³ or 10⁵ between before and after alteration.

In another embodiment of the invention, the alteration is or results in or is associated with a decrease of the mass or weight of the particle. In some cases, this decrease is a decrease from a mass or weight of the initial particle larger than 10⁻⁵⁰, 10⁻¹⁰, 10⁻⁵, 10⁻¹, 1, 5, 10, 100 or 10⁵ mg of particle or mg of particle per cm³ of assembly of particle or mg of particle per cm³ of body part or altering medium, preferentially before or without alteration, down to a mass or weight of the altered particle smaller than 10¹⁰⁰, 10⁵⁰, 10¹⁰, 10⁵, 10², 10, 5, 2, 1, 10⁻⁵ or 10⁻¹⁰ mg of particle or mg of particle per cm³ of assembly of particle or mg of particle per cm³ of body part or altering medium, preferentially after, during or with alteration. In some other cases, this decrease is a decrease by at least 10⁻⁵⁰, 10⁻¹⁰, 10⁻⁵, 10⁻¹, 1, 5, 10, 25, 50, 70 or 90% between before and after alteration. This percentage can equal to abs(Ma−Mi)/Mi, Ma/Mi, where Ma and Mi are the mass or weight of the altered and initial particles, respectively.

In another embodiment of the invention, the alteration is or results in or is associated with an increase of the mass or weight of the particle. In some cases, this increase is an increase from a mass or weight of the initial particle lower than 10⁵⁰, 10¹⁰, 10⁵, 10, 5, 1, 10⁻¹ or 10⁻⁵ mg of particle or mg of particle per cm³ of assembly of particle or mg of particle per cm³ of body part or altering medium, preferentially before or without alteration, up to a mass or weight of the altered nanoparticle larger than 10⁻¹⁰⁰, 10⁻⁵⁰, 10⁻¹⁰, 10⁻², 0, 1, 5, 10, 10⁵ or 10¹⁰ mg of particle or mg of particle per cm³ of assembly of particle or mg of particle per cm³ of body part or altering medium, preferentially during, after or with alteration.

In some other cases, this increase is an increase by at least 10⁻⁵⁰, 10⁻¹⁰, 10⁻⁵, 10⁻¹, 1, 5, 10, 25, 50, 70 or 90% between before and after alteration. This percentage can equal to abs(Ma−Mi)/Ma, Mi/Ma, where Ma and Mi are the mass or weight of the altered and initial particles, respectively.

As an example, table 6 shows the changes in sizes of the nanoparticles between before and after alteration, which can result in a change in mass or weight of the nanoparticles.

In some cases, a change in size of the particle by at least 10⁻⁵⁰, 10⁻¹⁰, 10⁻⁵, 10⁻¹, 1, 5, 10, 25, 50, 70 or 90% between before and after alteration can result in a change in mass or weight of the particle by at least 10⁻⁵⁰, 10⁻¹⁰, 10⁻⁵, 10⁻¹, 1, 5, 10, 25, 50, 70 or 90% between before and after alteration. These percentages can be equal to S2/S1, (S2−S1)/S1, S1/S2, (S1−S2)/S1, M2/M1, (M2−M1)/M1, M1/M2, (M1−M2)/M2, where the S1, S2, M1, and M2, are the size, mass or weight, before (number 1) and after (number 2) alteration. This may be the case when the change in nanoparticle size has a direct effect on nanoparticle mass or weight, i.e. preferentially induces a loss or gain of mass or weight of the nanoparticles.

In some other cases, a change in size of the particle by at least 10⁻⁵⁰, 10⁻¹⁰, 10⁻⁵, 10⁻¹, 1, 5, 10, 25, 50, 70 or 90% between before and after alteration can result in a change in mass or weight of the particle by less than 10¹⁰, 100, 90, 70, 50, 10, 5, 2, 1 or 10⁻⁵% between before and after degradation. This may be the case when the change in particle size does not have a direct effect on particle mass or weight In an embodiment of the invention, the alteration is or results in or is associated with a modification of the magnetic properties of the particle. Such change can be a change: i) from a diamagnetic property of the initial particle to a paramagnetic, superparamagnetic, ferromagnetic, and/or ferromagnetic property of the altered particle, ii) from a paramagnetic property of the initial particle to a diamagnetic, superparamagnetic, ferromagnetic, and/or ferromagnetic property of the altered particle, iii) from a superparamagnetic property of the initial particle to a diamagnetic, paramagnetic, ferromagnetic, and/or ferromagnetic property of the altered particle, iv) from a ferromagnetic property of the initial particle to a diamagnetic, paramagnetic, superparamagnetic, and/or ferromagnetic property of the altered particle, and/or v) from a ferromagnetic property of the initial particle to a diamagnetic, paramagnetic, superparamagnetic, and/or ferromagnetic property of the altered particle.

In another embodiment of the invention, the modification of the magnetic properties of the particle is an increase of at least one of the following magnetic parameters: i) the coercivity of the particle, preferentially from a coercivity of the initial particle lower than 10⁵⁰, 10¹⁰, 10⁵, 10³, 10, 1, 10⁻¹, 10⁻⁵ or 10⁻¹⁰ Oe, preferentially before or without alteration, up to a coercivity of the altered particle larger than 10⁻¹⁰, 10⁻⁵, 10⁻¹, 1, 5, 10, 10³, 10⁵, 10¹⁰ or 10⁵⁰ Oe, preferentially during, after or with alteration, ii) the remanent magnetization of the particle, preferentially from a remanent magnetization of the initial particle lower than 1, 0.99, 0.9, 0.8, 0.7, 0.6, 0.5, 0.4, 0.3, 0.2 or 0.1, preferentially measured before or without alteration, up to a remanent magnetization of the altered particle larger than 10⁻⁵⁰, 10⁻¹⁰, 10⁻⁵, 10⁻¹, 0.1, 0.2, 0.3, 0.4, 0.5, 0.6, 0.7, 0.8 or 0.9, during, after or with alteration, iii) the saturating magnetization of the particle, from a saturating magnetization of the initial particle lower than 10⁵⁰, 10²⁰, 10¹⁰, 10⁵, 10³, 10, 5, 2, 1, 10⁻², 10⁻⁵, 10⁻¹⁰ or 10⁻⁵⁰ emu or emu per gram or milligram of particle, preferentially before or without alteration, up to a saturating magnetization of the altered particle larger than 10⁻⁵⁰, 10⁻²⁰, 10⁻¹, 10⁻¹, 1, 5, 10, 100, 10⁵ or 10¹⁰ emu or emu per gram or milligram of particle, preferentially during, after or with alteration. In some cases, the magnetic parameters can exist or be measured at a temperature larger than 0, 0.1, 5, 10, 10³, 10⁵, 10¹⁰ or 10²⁰ K (Kelvin). In some other cases, the magnetic parameters can exist or be measured at temperatures lower than 10¹⁰⁰, 10⁵⁰, 10²⁰, 10¹⁰, 10⁵, 10³, 100, 50, 20, 10, 5, 2, 1 or 0.1 K.

In another embodiment of the invention, the modification of the magnetic properties of the particle is a decrease of at least one of the following magnetic parameters: i) the coercivity of the particle, preferentially from a coercivity of the initial particle larger than 10⁻¹⁰, 10⁻⁵, 10⁻¹, 1, 5, 10, 10³, 10⁵, 10¹⁰ or 10⁵⁰ Oe, preferentially before or without alteration, down to a coercivity of the altered particle lower than 10⁵⁰, 10¹⁰, 10⁵, 10³, 10, 1, 10⁻¹, 10⁻⁵ or 10⁻¹⁰ Oe, preferentially after or with alteration, ii) the remanent magnetization of the particle, preferentially from a remanent magnetization of the initial particle larger than 10⁻⁵⁰, 10⁻¹⁰, 10⁻⁵, 10⁻¹, 0.1, 0.2, 0.3, 0.4, 0.5, 0.6, 0.7, 0.8 or 0.9, preferentially before or without alteration, down to a remanent magnetization of the altered particle smaller than 1, 0.99, 0.9, 0.8, 0.7, 0.6, 0.5, 0.4, 0.3, 0.2 or 0.1, preferentially during, after or with alteration, iii) the saturating magnetization of the particle, preferentially from a saturating magnetization of the initial particle larger than 10⁻⁵⁰, 10⁻²⁰, 10⁻¹⁰, 10⁻¹, 1, 5, 10, 100, 10⁵ or 10¹⁰ emu or emu per gram or milligram of particle, preferentially before or without alteration, down to a saturating magnetization of the altered particle smaller than 10⁵⁰, 10²⁰, 10¹⁰, 10⁵, 10³, 100, 10, 5, 2, 1, 10⁻², 10⁻⁵, 10⁻¹⁰ or 10⁻⁵⁰ emu or emu per gram or milligram of particle, preferentially during, after or with alteration.

As an example, table 6 shows that the magnetosome size can decrease between before and after alteration. Such size decrease can result in a change of the magnetic properties of the magnetosomes, preferentially a change from a ferrimagnetic to a superparamagnetic behavior, or a significant variation, preferentially a decrease, of the coercivity, and/or remanent magnetization of the magnetosomes.

In one embodiment of the invention, the alteration is or results in or is associated with a modification of a property of assembly, organization, and/or distribution of the nanoparticles. Such modification can be selected from the group consisting of: i) an organization of initial particle in chains, preferentially existing or measured before or without alteration, to an organization of altered particle that is not in chains, preferentially existing or measured during, after, or with alteration, ii) an organization of initial particle in aggregates, preferentially existing or measured before or without alteration, to an organization of altered particle that is not in aggregates, preferentially existing or measured during, after or with alteration, iii) an organization of initial particle in a geometric figure such as a circle, preferentially existing or measured before or without alteration, to an organization of altered particle that is not in a geometric figure, preferentially existing or measured during, after or with alteration, and/or iv) an homogenous distribution of the initial particle, preferentially existing or measured before or without alteration, to a non-homogenous distribution of the altered particle, preferentially existing or measured during, after or with alteration.

In one embodiment of the invention, the modification of a property of assembly, organization, and/or distribution of the particle is selected from the group consisting of: i) an organization of initial particle that is not in chains, preferentially existing or measured before or without alteration, to an organization of altered particle in chains, preferentially existing or measured during, after or with alteration, ii) an organization of initial particle that is not in aggregates, preferentially existing or measured before or without alteration, to an organization of altered particle in aggregates, preferentially existing or measured during, after or with alteration, iii) an organization of initial particle that is not in a geometric figure, preferentially existing or measured before or without alteration, to an organization of altered particle in a geometric figure, preferentially existing or measured during, after or with alteration, and/or iv) a non-homogenous distribution of the initial particle, preferentially existing or measured before or without alteration, to a homogenous distribution of the altered particle, preferentially existing or measured during, after or with alteration.

In another embodiment of the invention, the alteration is or results in or is associated with a modification of the crystallinity of the particle. In some cases, such modification can be a change from a crystalline condition, preferentially observed or existing in the initial particle, preferentially before or without alteration, to an amorphous condition of the particle, preferentially observed or existing in the altered particle, preferentially after, during, or with alteration.

In another embodiment of the invention, the modification of the crystallinity of the particle is a change from an amorphous condition of the initial particle, preferentially observed or existing before or without alteration, to a crystalline condition of the altered particle, preferentially observed or existing during, after or with alteration.

In some cases, a crystalline condition can be characterized by the presence of more than 1, 2, 5, 10, 10³ or 10⁵ crystallographic plane(s) or ordered atom(s), preferentially comprised in the particle, preferentially observable by electron microscopy.

In some other cases, an amorphous condition can be characterized by the presence of less than 10⁵⁰, 10²⁰, 10¹⁰, 10⁵, 10³, 100, 50, 20, 10, 5, 2 or 1 crystallographic plane(s) or ordered atom(s), preferentially comprised in the particle, preferentially observable by electron microscopy.

In another embodiment of the invention, the modification of the crystallinity of the particle is an increase in the number of crystallographic planes or ordered atoms, preferentially per particle, preferentially from less than 10¹⁰⁰, 10⁵⁰, 10¹⁰, 10⁵, 10³, 10, 5, 2 or 1 crystalline planes in the initial particle, preferentially existing or measured before or without alteration, up to more than 1, 5, 10, 10³, 10⁵ or 10¹⁰ crystalline planes in the altered particle, preferentially existing or measured during, after or with alteration.

In still another embodiment of the invention, the modification of the crystallinity of the particle is a decrease in the number of crystallographic planes or ordered atoms, preferentially per particle, preferentially from more than 1, 5, 10, 10³, 10⁵ or 10¹⁰ crystallographic planes in the initial particle, preferentially existing or measured before or without alteration, down to less than 10¹⁰⁰, 10⁵⁰, 10¹⁰, 10⁵, 10³, 10, 5, 2 or 1 crystallographic planes in the altered particle, preferentially existing or measured during, after or with alteration.

As an example, tables 6 shows the change in size of the particle between before and after alteration, which can result in a decrease in the number of crystalline planes per particle, preferentially by a factor of more than 1.0001, 1.2, 1.5, 2, 5, 10 or 10³ between before and after alteration.

In still another embodiment of the invention, the modification of the crystallinity of the particle is a change: i) from a crystalline to an amorphous composition of the particle, ii) a change from an amorphous to a crystalline composition of the particle, iii) a change from an amorphous or crystalline composition of the particle to an ionic composition of the particle, iv) a change from an ionic composition of the particle to an amorphous or crystalline composition of the particle, v) a change from a solid to liquid composition of the particle, vi) a change from a solid to a gaseous composition of the particle, vii) a change from a liquid to a solid composition of the particle, viii) a change from a liquid to a gaseous composition of the article, ix) a change from a gaseous to a liquid composition of the particle, x) a change from a gaseous to a solid composition of the particle.

In one embodiment of the invention, the alteration is or results in or is associated with a modification of the number or concentration of nanoparticles. Such modification can be a decrease from more than 10⁻⁵⁰, 10⁻²⁰, 10⁻¹⁰, 10⁻⁵, 10⁻², 10⁻¹, 1, 5, 10, 10³ or 10⁵ initial nanoparticles or mg of initial nanoparticles, preferentially per cm³ of body part or altering medium, preferentially existing or measured before or without alteration, to less than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ altered nanoparticles or mg of altered nanoparticles, preferentially per cm³ of body part or altering medium, preferentially existing or measured during, after or with alteration.

In some other cases, the modification of the number or concentration of nanoparticles can be an increase from less than 10¹⁰⁰, 10⁵⁰, 10²⁰, 10¹⁰, 10⁵, 10², 10, 5, 2, 1, 10⁻¹, 10⁻⁵, 10⁻¹⁰ or 10⁻⁵⁰ initial nanoparticles or mg of initial nanoparticles, preferentially per cm³ of body part, preferentially existing or measured before or without alteration, up to more than 10⁻⁵⁰, 10⁻²⁰, 10⁻¹⁰, 10⁻⁵, 10⁻², 10⁻¹, 1, 5, 10, 10³ or 10⁵ altered nanoparticles or mg of altered nanoparticles, preferentially per cm³ of body part, preferentially existing or measured during, after or with alteration.

In one embodiment of the invention, the alteration is or results in or is associated with a modification of the morphology or geometry or of particle, preferentially the nanoparticle. Such a modification can be a change from a geometric figure or morphology before particle alteration to another geometric figure or morphology after particle alteration. The different types of geometric figures were listed before.

In some cases, the observed or existing morphologies of the particle can comprise cubo-octahedric, elongated, octahedral, prismatic, bullet, equidimensional, or irregular morphologies.

In some other cases, while before alteration, at least one geometry or morphology such as the cubo-octahedric geometry can be a frequent or the most morphology, as observed for magnetosomes in FIG. 1(b) of the experimental section, at least one other geometry or morphology such as the cubic geometry can be a frequent or the most frequent morphology after alteration such as cellular alteration, as observed for altered magnetosomes in FIGS. 2(a) and 2(b) of the experimental section.

In one embodiment of the invention, the alteration is or results in or is associated with a modification of the number of facets of the particle, preferentially nanoparticle. In some cases, a facet can be a flat face of the particle, preferentially nanoparticle, which can preferentially be observed by electron microscopy.

In one embodiment of the invention, the modification of the number of facets or edges or corners of the particle, preferentially nanoparticle, is a decrease from a number of facets or edges or corners that is larger than 1, 2, 5, 10, 10³ or 10⁵ facets or edges or corners, preferentially per particle, preferentially per mg of particle, preferentially per cm³ of body part down to a number of facets or edges or corners that is smaller than 10⁵⁰, 10⁵, 10³, 10, 5, 2 or 1, preferentially per particle, preferentially per mg of particle, preferentially per cm³ of body part.

In one embodiment of the invention, the modification of the number of facets or edges or corners of the particle, preferentially nanoparticle, is an increase from a number of facets or edges or corners that is smaller than 10⁵⁰, 10⁵, 10³, 10, 5, 2 or 1, preferentially per particle, preferentially per mg of particle, preferentially per cm³ of body part up to a number of facets or edges or corners that is larger than 1, 2, 5, 10, 10³ or 10⁵, preferentially per particle, preferentially per mg of particle, preferentially per cm³ of body part.

In one embodiment of the invention, the number of facets in particle, preferentially nanoparticle, is larger before than after alteration, as can observed in the experimental section by comparing the magnetosomes morphologies of FIG. 1(b) with those of FIGS. 2(a) and 2(b).

In one embodiment of the invention, the alteration is, or results in, or produces or is associated with a modification of the faculty of the particle, preferentially nanoparticle, to release at least one compound, also designated as efficacy of the release mechanism. In some cases, the efficacy of the release mechanism can increase between before and after alteration, preferentially when the number or concentration of released compounds increases, preferentially by a factor larger than 1.001, 1.1, 1.5, 5, 10, 10², 10³ or 10⁵.

In another embodiment of the invention, the alteration is, or results in, or produces or is associated with a modification of the quantity of heat produced or absorbed by the particle, preferentially nanoparticle, preferably under the application of a radiation. In some cases, such quantity of heat can be designated as specific absorption rate (SAR).

In some cases, the SAR of the particle can decrease from a SAR that is larger than 10⁻⁵⁰, 10⁻¹⁰, 10⁻¹, 1, 5, 10, 100 or 500 Watt per gram of particle in the initial particle, preferentially before or without alteration, down to a SAR that is lower than 10⁵⁰, 10¹⁰, 10⁵, 10³, 10, 5, 2 or 1 Watt per gram of particle in the altered particle, preferentially during, after or with alteration. In some other cases, the SAR of the particle can decrease by factor of more than 1.001, 1.1, 1.5, 5, 10, 10³ or 10⁵ between before and after alteration. In still some other cases, the amount of heat produced by the particle, preferentially under exposure to radiation, can decrease, preferentially from a value that is above 0, 1, 2, 5, 10, 10³ or 10⁵° C. in the initial particle, preferentially before or without alteration, down to a value that is lower than 10⁵, 10³, 10, 5, 2 or 1° C. (degree Celsius) or that is equal to 0° C. in the altered particle, preferentially during, after or with alteration.

As an example, FIG. 4(b) shows that the temperature increase produced by magnetosomes introduced to mouse tumors and exposed to an alternating magnetic field decreases from 4° C. (at the day of magnetosome injection where magnetosome alteration is limited) down to 0° C. (9 days after magnetosome injection when magnetosome alteration is more pronounced).

In some cases, the SAR of the particle can increase from a SAR that is lower than 10⁵⁰, 10¹⁰, 10⁵, 10³, 10, 5, 2 or 1 Watt per gram of particle in the initial particle, preferentially before or without alteration up to a SAR that is larger than 10⁻⁵⁰, 10⁻¹⁰, 10⁻¹, 1, 5, 10, 100 or 500 Watt per gram of particle in the altered particle, preferentially during, after or with alteration. In some other cases, the SAR of the particle can increase by factor of more than 1.001, 1.1, 1.5, 5, 10, 10³ or 10⁵ between before and after alteration. In still some other cases, the amount of heat produced by the particle, preferentially under exposure to radiation, can increase, from a value that is lower than 10⁵, 10³, 10, 5, 2 or 1° C. (degree Celsius) or that is equal to 0° C. in the initial particle, preferentially before or without alteration, up to a value that is above 0, 1, 2, 5, 10, 10³ or 10⁵° C. in the altered particle, preferentially during, after or with alteration.

In another embodiment of the invention, the alteration is, or results in, or produces or is associated with a modification of the quantity of radical or reactive species produced by the particle, preferably under the application of a radiation.

In some cases, the quantity of radical or reactive species produced by the particle varies between before and after alteration by a magnitude of at least 10⁻¹⁰, 10⁻⁵, 10⁻¹, 1, 5, 10, 10³ or 10¹⁰ μM of radical or reactive species, preferentially per particle or per milligram of particle or per cm³ of particle or per cm³ of body part.

In some other cases, the quantity of radical or reactive species produced by the particle increases between before and after alteration from a quantity of radical or reactive species produced by the initial particle that is lower than 10¹⁰, 10⁵, 10, 1, 10⁻¹, 10⁻³ or 10⁻¹⁰ μM of radical or reactive species, preferentially per particle or per milligram of particle or per cm³ of particle or per cm³ of body part up to a quantity of radical or reactive species produced by the altered particle that is larger than 10⁻¹⁰, 10⁻⁵, 10⁻¹, 1, 5, 10, 10³ or 10¹⁰ μM of radical or reactive species, preferentially per particle or per milligram of particle or per cm³ of particle or per cm³ of body part.

In still some other cases, the quantity of radical or reactive species produced by the particle decreases between before and after alteration from a quantity of radical or reactive species produced by the initial particle that is that is larger than 10⁻¹⁰, 10⁻⁵, 10⁻¹, 1, 5, 10, 10³ or 10¹⁰ μM of radical or reactive species, preferentially per particle or per milligram of particle or per cm³ of particle or per cm³ of body part down to a quantity of radical or reactive species produced by the altered particle that is lower than 10¹⁰, 10⁵, 10, 1, 10⁻¹, 10⁻³ or 10⁻¹⁰ μM of radical or reactive species, preferentially per particle or per milligram of particle or per cm³ of particle or per cm³ of body part.

In some cases, the reactive or radical species can be made of reactive oxygen species (ROS) and/or reactive nitrogen species (RNS). Examples of ROS and RNS include superoxide, oxygen radical, hydroxyl, alkoxyradical, peroxyl radical, nitric oxide, nitrogen monoxide, and nitrogen dioxide.

In one embodiment of the invention, the alteration is, results in, or is associated with a variation or change of at least one property of the particle. In some cases, such change can be larger than 10⁻⁵⁰, 10⁻¹⁰ 10⁻⁵, 10⁻¹, 1, 5, 10, 50, 80, 10², 10³, 10⁵ or 10¹⁰%. This percentage may be equal to (P1−P2)/P1, P1/P2, (P2−P1)/P1, or P2/P1, where P1 and P2 are particle properties before and after alteration, respectively. In some other cases, such change can be lower than 10⁵⁰, 10¹⁰, 10⁵, 10, 5, 1 or 10⁻⁵%.

In one embodiment of the invention, the alteration generates, leads to, results in, is associated with, is, produces, or yields a first partial release generating a first part of altered compounds released from the altered nanoparticle.

In some cases, the first part of altered compounds is or represents the compounds released by alteration. The first part of said altered compounds can be unbound from the altered nanoparticle after alteration.

In some cases, the first partial release can generate, lead to, be, result in, lead to, be due to, be associated with, yield, or originate from the breaking, preferentially complete breaking, of the altered bond between the first part of the altered compound and the altered nanoparticle. In some cases, the complete breaking can be the breaking of all bonds between the altered compound and the altered nanoparticle or the breaking of the bonds resulting in the location of the altered compound at a distance larger than 10⁻¹, 1, 10, 10², 10³ or 10⁵ nm from the altered nanoparticle. In some cases, the breaking of the altered bond between the altered compound and the altered nanoparticle can be or be designated as the breaking of the bond or the first partial release.

In some cases, the breaking of the bond between the nanoparticle, preferentially altered, and the compound, preferentially altered, can be the removal of the bond, link, or interaction between the nanoparticle and the compound, preferentially resulting in the compound: i) diffusing freely in the environment of the particle, or ii) located at a distance from the nanoparticle that has increased by a factor of at least 1.001, 1.1, 1.5, 2, 5, 10, 10³ or 10⁵ between before and after alteration.

In some cases, the first part of altered compounds can result from, come from, originate from, be produced by, or be generated by the first partial release of the first part of altered compounds from the altered nanoparticle.

In some cases, the first part of altered compounds can be the quantity or concentration of compounds that are released from the altered nanoparticle, preferentially following alteration. In some other cases, the first part of altered compounds can be bound to the initial nanoparticle, preferentially before alteration.

In still some other cases, the first part of altered compounds is not bound to the altered nanoparticle, preferentially after alteration.

In some cases, the first part of altered compounds is or represents a number or concentration of compounds, preferentially released altered compounds, larger than: i) 0, 1, 5, 10, 10³, 10⁵, 10¹⁰, 10²⁰ or 10⁵⁰ compound(s), preferentially released altered compounds, preferentially per altered nanoparticle, or ii) more than 0, 10⁻⁵⁰, 10⁻¹⁰, 10⁻¹, 1, 5, 10, 50, 75, 80, 90 or 99% of the initial compounds initially bound to the initial nanoparticle. This percentage can be equal to N_{ACR}/N_{IC}, where N_{ACR} is the number or concentration of altered compounds released from the altered nanoparticle and N_{IC} is the number of concentration of initial compounds bound to the initial nanoparticle.

In some other cases, the first part of altered compounds is or represents a number or concentration of compounds, preferentially released altered compounds, smaller than: i) 10⁵⁰, 10²⁰, 10¹⁰, 10⁵, 10³, 10, 5 or 1 compound(s), preferentially released altered compounds, preferentially per altered nanoparticle, or ii) less than 100, 99, 80, 70, 50, 20, 10, 5, 2 or 1% of the initial compounds initially bound to the initial nanoparticle.

In one embodiment of the invention, the alteration generates, results in, leads to, is associated with, produces or yields an absence of release, generating a second part of altered compounds bound to the altered nanoparticle and/or not released from the altered nanoparticle. In some cases, such absence of release can be designated as the absence of release by alteration.

In some cases, the second part of altered compounds is or represents the altered compounds not-released by alteration. The second part of said altered compound can remain bound to the altered nanoparticle after alteration.

In some cases, the absence of release by alteration can generate, lead to, be, result in, lead to, be due to, be associated with, yield, or originate from the absence of breaking of the altered bond between the second part of altered compounds and the altered nanoparticle. In some cases, the absence of breaking can be the absence of breaking of all bonds between the altered compound and the altered nanoparticle or the absence of breaking of the bonds resulting in the location of the altered compound at a distance smaller than 10⁻¹, 1, 10, 10², 10³ or 10⁵ nm from the altered nanoparticle. In some cases, the absence of breaking of the altered bond between the altered compound and the altered nanoparticle can be or be designated as the absence of breaking of the bond or the absence of release by alteration.

In some cases, the absence of breaking of the bond between the nanoparticle, preferentially altered, and the compound, preferentially altered, can be the absence of removal of the bond, link, or interaction between the nanoparticle and the compound, preferentially resulting in the compound: i) remaining bound to the nanoparticle, ii) not diffusing freely in the environment of the particle, or iii) located at a distance from the nanoparticle that has increased by a factor of less than 1.001, 1.1, 1.5, 2, 5, 10, 10³ or 10⁵ between before and after alteration.

In some cases, the absence of release by alteration can result from, come from, originate from, be produced by, or be generated by the absence of release of the second part of altered compounds from the altered nanoparticle.

In some cases, the second part of altered compounds can be the quantity or concentration of compounds that: i) are not released from the altered nanoparticle, preferentially following alteration, ii) or remain bound to the altered nanoparticle, preferentially following alteration. In some other cases, the second part of altered compounds can be bound to the initial nanoparticle, preferentially before alteration.

In still some other cases, the second part of altered compounds can be bound to the altered nanoparticle, preferentially after alteration.

In some cases, the second part of altered compounds can be or represent a number or concentration of compounds, preferentially non-released altered compounds, larger than: i) 0, 1, 5, 10, 10³, 10⁵, 10¹⁰, 10²⁰ or 10⁵⁰ compound(s), preferentially non-released altered compounds, preferentially per altered nanoparticle, or ii) more than 10⁻⁵⁰, 10⁻¹⁰, 10⁻¹, 1, 5, 10, 50, 75, 80, 90 or 99% of the initial compounds initially bound to the initial nanoparticle. This percentage can be equal to N_{ANR}/N_{IC}, where N_{ANR} is the number or concentration of altered compounds not released from the altered nanoparticle and N_{IC} is the number of concentration of initial compounds bound to the initial nanoparticle.

In some other cases, the second part of altered compounds can be or represent a number or concentration of compounds, preferentially non-released altered compounds, smaller than: i) 10⁵⁰, 10²⁰, 10¹⁰, 10⁵, 10³, 10, 5 or 1 compound(s), preferentially non-released altered compounds, preferentially per altered nanoparticle, or ii) less than 100, 99, 80, 70, 50, 20, 10, 5, 2 or 1% of the initial compounds initially bound to the initial nanoparticle.

In some cases, the second part of altered compounds can be at least 1.01, 1.1, 1.5, 2, 5, 10, 10³ or 10⁵ larger than the first part of said altered compounds. This can be the case when the alteration does not lead to an efficient first partial release.

In some other cases, the second part of altered compounds can be at least 1.01, 1.1, 1.5, 2, 5, 10, 10³ or 10⁵ smaller than the first part of altered compounds. This can be the case when the alteration leads to an efficient first partial release.

In still some other cases, the sum of the first part of altered compounds and second part of altered compounds represents or is the total number of compounds initially bound to the initial nanoparticle.

In some cases, the total number of compounds initially bound to the initial nanoparticle is the number of compounds bound to the nanoparticle before or without alteration.

Preferably, the method for increasing the release of at least one compound comprises a physico-chemical disturbance chosen among:

i) a variation of the environment of the altered particle comprising the altered nanoparticle and/or the altered compound and/or altered bond, and/or
ii) a radiation applied on said altered particle.

In some cases, the physico-chemical disturbance can be the change of the state or condition of the particle from an altered state or altered condition of the particle in which the particle is the altered particle to an altered and disturbed state or an altered and disturbed condition of the particle in which the particle is the altered and disturbed particle.

In some other cases, the physico-chemical disturbance can be the change of the state or condition of the particle from an initial state or initial condition of the particle in which the particle is the initial particle to a disturbed state or a disturbed condition of the particle in which the particle is the disturbed particle.

In some cases, the physico-chemical disturbance can be the physico-chemical disturbance of the initial particle, the physico-chemical disturbance of the altered particle, and/or the physico-chemical disturbance of the altered and disturbed particle.

Preferentially, this invention shows that the physico-chemical disturbance generating the change from the altered to altered and disturbed particle is more efficient or generates a more important number of compounds released from the nanoparticle than the physico-chemical disturbance generating the change from the initial particle to the disturbed particle, i.e. without the step of alteration of the particle.

In one embodiment of the invention, a physico-chemical disturbance is applied on said altered particle, resulting in, leading to, being associated with, producing or yielding the formation of an altered and disturbed particle. In some cases, an altered and disturbed particle can be an altered particle that is disturbed. In some cases, the altered particle that is exposed to the physico-chemical disturbance is the altered and disturbed particle. In some cases, the altered nanoparticle that is exposed to a physico-chemical disturbance is the altered and disturbed nanoparticle. In some cases, the altered compound that is exposed to a physico-chemical disturbance is the altered and disturbed compound. In some cases, the bond between the altered compound and the altered nanoparticle that is exposed to a physico-chemical disturbance is the altered and disturbed bond.

In one embodiment of the invention, the application of a physico-chemical disturbance on said particle, preferentially the altered or altered and disturbed particle, is the exposure of said particle, preferentially the altered or altered and disturbed particle, to a physico-chemical disturbance. In some cases, the application of a physico-chemical disturbance on said particle can be designated as the physico-chemical disturbance.

In some cases, the physico-chemical disturbance and/or alteration of the particle can be the treatment of the particle.

In one embodiment of the invention, the altered and disturbed particle comprise at least one active ingredient, which is in some cases the same as that comprised in the altered particle, which is some other cases different from that comprised in the altered particle.

In one embodiment of the invention, the application of a physico-chemical disturbance on said second part of said altered compound not released by alteration is divided between: i) a group 1 of said second part of the compound not released after alteration and not released after physico-chemical disturbance and ii) a group 2 of said second part of the compound released following successive alteration and physico-chemical disturbance. In some cases, the group mentioned in i) can be designated as the group 1 of the second part. In some other cases, the group mentioned in ii) can be designated as the group 2 of the second part.

In one embodiment of the invention, the application of a physico-chemical disturbance on said particle, preferentially the altered and disturbed particle, is associated with, yields, results in, produces, or leads to an absence of release, preferentially of altered and disturbed compounds, preferentially of compounds originating from or of the second part of altered compounds not released by alteration or of compounds not released by alteration. In some cases, such absence of release is designated as the absence of release by physico-chemical disturbance. Such absence of release can be, yield, result in, generate, or be associated with: i) non-released altered and disturbed compounds belonging to group 1 of the second part, or ii) the appearance, the maintenance, the existence, of non-released altered and disturbed compounds belonging to group 1 of the second part. In some cases, such absence of release can be the absence of release of the compound from the nanoparticle that has not been released by the alteration, and that is not further released by the physico-chemical disturbance. In some cases, the altered and disturbed compound belonging to group 1 of the second part of compounds is not released by physico-chemical disturbance. In some cases, the compound belonging to group 1 of the second part can remain bound to the altered and disturbed nanoparticle following the successive alteration and physico-chemical disturbance.

In one embodiment of the invention, non-released altered and disturbed compounds belonging to group 1 of the second part are not released by, after, or during the alteration and are not released by, after, or during the application of the physico-chemical disturbance. In some cases, these compounds belonging to group 1 are the compounds, which are: i) strongly or the most strongly bound to the altered and disturbed nanoparticle, ii) in strong interactions with the altered and disturbed nanoparticle, iii) covalently linked to the altered and disturbed nanoparticle, iv) bound to the altered and disturbed nanoparticle via a binding dissociation energy that is larger by a factor of at least 1.001, 1.1, 1.5, 2, 5, 10, 10³, 10⁵ or 10¹⁰ than the binding dissociation energy of the compounds belonging to group 2 of the second part.

In some cases, the absence of release by physico-chemical disturbance can be due to the absence of breaking of the altered and disturbed bond between the altered and disturbed nanoparticle and the group 1 of the second part of altered and disturbed compounds.

In one embodiment of the invention, the application of a physico-chemical disturbance on said altered particle, preferentially the altered and disturbed particle, yields, results in, produces, or leads to a second partial release, preferentially of altered and disturbed compounds, preferentially of said second part of said compounds not released by alteration. In some cases, such second partial release can be, yield, result in, generate, or be associated with the release of altered and disturbed compounds belonging to group 2 of the second part from the altered and disturbed nanoparticle. In some cases, the second partial release is the release of the compound from the nanoparticle that has not been released by the alteration, but is released by the physico-chemical disturbance. In some cases, the altered and disturbed compound belonging to group 2 of the second part of compounds is released by physico-chemical disturbance. In some cases, the compound belonging to group 2 of the second part can be released by, after, or during the successive alteration and physico-chemical disturbance.

In one embodiment of the invention, the compound belonging to group 2 of the second part that is preferentially partially released is the compound, which is: i) weakly bound to the altered and disturbed nanoparticle or more weakly bound to the altered and disturbed nanoparticle than the compound of group 1 of the second part, ii) in weak interactions with the altered and disturbed nanoparticle, iii) weakly linked to the nanoparticle, such as being adsorbed at the surface of the altered and disturbed nanoparticle or not being covalently linked to the altered and disturbed nanoparticle, iv) bound to the altered and disturbed nanoparticle via a binding dissociation energy that is lower by a factor of at least 1.001, 1.1, 1.5, 2, 5, 10, 10³, 10⁵ or 10¹⁰ than the binding dissociation energy of the compounds belonging to group 1 of the second part.

In some cases, the said second partial release by physico-chemical disturbance can be associated with, lead to, result in, yield, produce, or due to the breaking of the altered and disturbed bond between the altered and disturbed nanoparticle and the group 2 of the second part of altered and disturbed compounds.

In an embodiment of the invention, the breaking of the bond between said altered and disturbed nanoparticle and said group 2 of the second part of the compound is or is designated as the breaking of the bond of the second partial release.

In one embodiment of the invention, the breaking of the bond of the second partial release can be more important or more efficient than the breaking of the bond of the first partial release. In this case, the number of bonds, preferentially per nanoparticle or compound, that is broken by or during the second partial release can be at least 1.01, 1.1, 1.2, 1.5, 2, 5, 10, 10³, 10⁵ or 10¹⁰ larger than the number of bonds, preferentially per nanoparticle or compound, that is broken by or during the first partial release.

In this case, the number of compounds, preferentially per nanoparticle or bond, that is released by or during the second partial release is at least 1.01, 1.1, 1.2, 1.5, 2, 5, 10, 10³, 10⁵ or 10¹⁰ larger than the number of compounds, preferentially per nanoparticle or bond, that is released by or during the first partial release.

In one embodiment of the invention, the breaking of the bond of the second partial release is less important or less efficient than the breaking of the bond of the first partial release. In this case, the number of bonds, preferentially per nanoparticle or compound, that is broken by or during the second partial release can be at least 1.01, 1.1, 1.2, 1.5, 2, 5, 10, 10³, 10⁵ or 10¹⁰ smaller than the number of bonds, preferentially per nanoparticle or compound, that is broken by or during the first partial release.

In this case, the number of compounds, preferentially per nanoparticle or bond, that is released by or during the second partial release is at least 1.01, 1.1, 1.2, 1.5, 2, 5, 10, 10³, 10⁵ or 10¹⁰ smaller than the number of compounds, preferentially per nanoparticle or bond, that is released by or during the first partial release.

In one embodiment of the invention, the breaking of the bond of the sum of the first and second partial release is more important or more efficient than the breaking of the bond of the first or second partial release, taken individually. In this case, the number of bonds, preferentially per nanoparticle or compound, that is broken by or during the sum of the first and second partial release can be at least 1.01, 1.1, 1.2, 1.5, 2, 5, 10, 10³, 10⁵ or 10¹⁰ larger than the number of bonds, preferentially per nanoparticle or compound, that is broken by or during the first or second partial release, taken individually. In this case, the number of compounds, preferentially per nanoparticle or bond, that is released by or during the sum of the first and second partial release is at least 1.01, 1.1, 1.2, 1.5, 2, 5, 10, 10³, 10⁵ or 10¹⁰ larger than the number of compounds, preferentially per nanoparticle or bond, that is released by or during the first or second partial release, taken individually.

As an example, the percentage of endotoxins, which can be considered as the compound, released from the magnetosomes following the application of a physico-chemical disturbance being an alternating magnetic field of average strength 27 mT, frequency 200 KHz, during 10 minutes, increases from 0.48% when magnetosomes are not degraded to 7.53% when magnetosomes are degraded with HCl, leading to a percentage of endotoxin release that increases by a factor of 16 between without and with degradation. In some cases, this factor can be larger, preferentially by a factor of at least 1.001, 1.1, 1.5, 2, 5, 10, 10³ or 10⁵ when the degradation is different from this HCl treatment or and enables the release of a more important number of compounds. In some other cases, this factor can be smaller, preferentially by a factor of at least 1.001, 1.1, 1.5, 2, 5, 10, 10³ or 10⁵ when the degradation is different from this HCl treatment and enables the release of a less important number of compounds.

In another embodiment of the invention, the application of a physico-chemical disturbance on said particle, preferentially the altered and disturbed particle, yields, results in, produces, or leads to a total release, preferentially of altered and disturbed compounds, preferentially of said entire second part of said compounds not released by alteration. In some cases, said total release can be, yield, result in, generate, or be associated with the release of all altered and disturbed compounds belonging to the entire second part from the altered and disturbed nanoparticle. In some cases, the total release is the release of the compound from the nanoparticle that has not been released by the alteration, but is released by the physico-chemical disturbance. In some cases, the altered and disturbed compound belonging to the entire second part of compounds is released by physico-chemical disturbance. In some cases, the compound belonging to the entire second part can be released by, after, or during the successive alteration and physico-chemical disturbance. In some cases, the entire second part of said compound can designate all compounds belonging to the entire second part that are all released after successive alteration and physico-chemical disturbance. In some cases, the total release can designate the release of all the compounds from the nanoparticles. In some cases, the total release can be achieved by a first partial release of the compound by the alteration followed by another release by physico-chemical disturbance, designed as total release, of all remaining compounds that have not been released by the alteration.

In one embodiment of the invention, the total release is, is associated with, leads to, produces, or corresponds to the breaking of the bond between said altered and disturbed nanoparticle and said entire second part of the compound.

In some cases, the breaking of the bond can be complete. A complete breaking of the bond can mean that the compound can't remain attached or bound to the nanoparticle following breaking of the bond. In one embodiment of the invention, the compound belonging the entire second part that is preferentially totally released is the compound, which is: i) weakly bound to the altered and disturbed nanoparticle or more weakly bond to the altered and disturbed nanoparticle than the compound of group 1 and/or group 2 of the second part, ii) in weak interactions with the altered and disturbed nanoparticle, iii) weakly linked to the nanoparticle, such as being adsorbed at the surface of the altered and disturbed nanoparticle or not being covalently linked to the altered and disturbed nanoparticle, and/or iv) bound to the altered and disturbed nanoparticle via a binding dissociation energy that is lower by a factor of at least 1.001, 1.1, 1.5, 2, 5, 10, 10³, 10⁵ or 10¹⁰ than the binding dissociation energy of the compounds belonging to group 1 and/or 2 of the second part.

In an embodiment of the invention, the breaking of the bond between said altered and disturbed nanoparticle and said entire second part of the compound is or is designated as the breaking of the bond of the total release.

In one embodiment of the invention, the breaking of the bond of the total release is more important or more efficient than the breaking of the bond of the first or second partial release. In this case, the number of bonds, preferentially per nanoparticle or compound, that is broken by or during the total release can be at least 1.01, 1.1, 1.2, 1.5, 2, 5, 10, 10³, 10⁵ or 10¹⁰ larger than the number of bonds, preferentially per nanoparticle or compound, that is broken by or during the first or second partial release. In this case, the number of compounds, preferentially per nanoparticle or bond, that is released by or during the total release is at least 1.01, 1.1, 1.2, 1.5, 2, 5, 10, 10³, 10⁵ or 10¹⁰ larger than the number of compounds, preferentially per nanoparticle or bond, that is released by or during the first or second partial release.

In one embodiment of the invention, the breaking of the bond of the total release is less important or less efficient than the breaking of the bond of the first partial release. In this case, the number of bonds, preferentially per nanoparticle or compound, that is broken by or during the total release can be at least 1.01, 1.1, 1.2, 1.5, 2, 5, 10, 10³, 10⁵ or 10¹⁰ smaller than the number of bonds, preferentially per nanoparticle or compound, that is broken by or during the first partial release. In this case, the number of compounds, preferentially per nanoparticle or bond, that is released by or during the total release is at least 1.01, 1.1, 1.2, 1.5, 2, 5, 10, 10³, 10⁵ or 10¹⁰ smaller than the number of compounds, preferentially per nanoparticle or bond, that is released by or during the first partial release.

In one embodiment of the invention, the breaking of the bond of the sum of the first and total release is more important or more efficient than the breaking of the bond of the first or total release, taken individually. In this case, the number of bonds, preferentially per nanoparticle or compound, that is broken by or during the sum of the first and total release can be at least 1.01, 1.1, 1.2, 1.5, 2, 5, 10, 10³, 10⁵ or 10¹⁰ larger than the number of bonds, preferentially per nanoparticle or compound, that is broken by or during the first or total release, taken individually. In this case, the number of compounds, preferentially per nanoparticle or bond, that is released by or during the sum of the first and total release is at least 1.01, 1.1, 1.2, 1.5, 2, 5, 10, 10³, 10⁵ or 10¹⁰ larger than the number of compounds, preferentially per nanoparticle or bond, that is released by or during the first or total release, taken individually.

In one embodiment of the invention, the breaking of the bond of the total release is more important or more efficient than the breaking of the bond of the first or second partial release, taken individually, or of the sum of the first and second partial releases. In this case, the number of bonds, preferentially per nanoparticle or compound, that is broken by or during the total release can be at least 1.01, 1.1, 1.2, 1.5, 2, 5, 10, 10³, 10⁵ or 10¹⁰ larger than the number of bonds, preferentially per nanoparticle or compound, that is broken by or during: i) the first or second partial release, taken individually, or ii) the sum of the first and second partial releases. In this case, the number of compounds, preferentially per nanoparticle or bond, that is released by or during the total release is at least 1.01, 1.1, 1.2, 1.5, 2, 5, 10, 10³, 10⁵ or 10¹⁰ larger than the number of compounds, preferentially per nanoparticle or bond, that is released by or during: i) the first or second partial release, taken individually, or ii) the sum of the first and second partial releases.

In one embodiment of the invention, the breaking of the bond of the total release is less important or less efficient than the breaking of the bond of the first or second partial release, taken individually, or of the sum of the first and second partial releases. In this case, the number of bonds, preferentially per nanoparticle or compound, that is broken by or during the total release can be at least 1.01, 1.1, 1.2, 1.5, 2, 5, 10, 10³, 10⁵ or 10¹⁰ smaller than the number of bonds, preferentially per nanoparticle or compound, that is broken by or during: i) the first or second partial release, taken individually, or ii) the sum of the first and second partial releases. In this case, the number of compounds, preferentially per nanoparticle or bond, that is released by or during the total release is at least 1.01, 1.1, 1.2, 1.5, 2, 5, 10, 10³, 10⁵ or 10¹⁰ smaller than the number of compounds, preferentially per nanoparticle or bond, that is released by or during: i) the first or second partial release, taken individually, or ii) the sum of the first and second partial releases.

In some cases, the particle can designate the initial, altered, or altered and disturbed particle.

In some cases, the initial particle can comprise at least one initial nanoparticle and at least one releasable initial compound, which is initially bound to said initial particle. Such compound can be bound to the initial nanoparticle, preferentially before or without alteration, and be released from the altered nanoparticle, preferentially during, after or with alteration.

In some cases, an altered particle comprising at least one altered nanoparticle and at least one releasable altered compound. Such compound can be can be bound to the initial nanoparticle, preferentially before or without alteration, and be released from the altered nanoparticle, preferentially during, after or with alteration.

In some cases, the obtaining of the altered particle can be, be associated with, correspond to the formation of the altered particle, starting from the initial particle, which is altered or exposed to the alteration.

In some other cases, the obtaining of the altered particle can be, be associated with or correspond to the formation of the altered particle, starting from the altered particle, which is altered or exposed to the alteration. In this case, at least two types of alteration or alterations can follow each other.

In some cases, the obtaining of an altered and disturbed particle can be, be associated with or correspond to the formation of the altered and disturbed particle, starting from the initial and/or altered and/or disturbed particle. In some cases, at least two types of physico-chemical disturbance can follow each other.

In one embodiment of the invention, the particle comprises: i) the nanoparticle, the bond, and the compound, ii) the nanoparticle and the compound, iii) the nanoparticle and the bond, iv) the bond and the compound, v) the nanoparticle, vi) the bond, or vii) the compound.

In one embodiment of the invention, the initial particle, initial compound, initial nanoparticle, and initial bond are the particle, compound, nanoparticle, and bound before or without the method according to the invention, or before or without step a) and/or b) of the method according to the invention. Preferentially, the initial compound is bound to the initial nanoparticle, preferentially via or through the initial bond, preferentially forming the initial particle. Preferentially, the initial particle is the particle comprising the initial nanoparticle and the initial compound, where the initial nanoparticle is preferentially bound to the initial compound via or through the initial bond.

In some cases, the initial nanoparticle can be bound to the initial compound when more than 1, 5, 10, 50, 75, 80, 90, 95, 99 or 100% of initial compounds are bound to the initial nanoparticle, where this percentage can be the ratio between the quantity of initial compounds bound to the initial nanoparticle divided by the total quantity of initial compounds (initial compounds bound to the initial nanoparticle and initial compounds unbound from the initial nanoparticle).

In some cases, the initial particle can be defined as a particle comprising an assembly of initial nanoparticles and initial compounds, where more than 1, 5, 10, 50, 75, 80, 90, 95, 99 or 100% of initial compounds are bound to at least one initial nanoparticle. This can for example be the case when a suspension comprises initial compounds bound to initial nanoparticles, and no or a low amount of initial compounds are found or observed in the supernate of this suspension, preferentially after the separation between the initial nanoparticles bound to the initial compounds and the supernate.

In some cases, the compound can be localized: i), at the surface of the nanoparticle, ii) in some cases at a distance larger than 10⁻¹, 1, 10, 10³ or 10⁵ nm from the nanoparticle, iii) in some other cases at a distance smaller than 10⁵⁰, 10¹⁰, 10⁵, 10³, 10 or 1 nm from the nanoparticle, iv) in the coating of the nanoparticle, and/or v) in the central part of the nanoparticle.

In some cases, the particle can comprise at least one property in common with the nanoparticle and/or bond.

In some other cases, the nanoparticle can comprise at least one property in common with the particle and/or bond.

In still some other cases, the bond can comprise at least one property in common with the particle and/or nanoparticle.

In some cases, the particle can comprise at least one property, which is different from one property of the nanoparticle and/or bond.

In some other cases, the nanoparticle can comprise at least one property, which is different from one property of the particle and/or bond.

In still some other cases, the bond can comprise at least one property, which is different from one property of the particle and/or nanoparticle.

In one embodiment of the invention, a(the) particle(s), a(the) compound(s), a(the) nanoparticle(s), a(the) bond(s) or a(the) ingredient(s) is/are or represent(s) more than or an assembly of more than 1, 10, 10³, 10⁵, 10¹⁰, 10⁵⁰ or 10¹⁰⁰ particle(s), compound(s), nanoparticle(s), a (the) bond(s) or ingredient(s), preferentially per nm³, mm³, cm³, or m³ preferentially of body part.

In another embodiment of the invention, a (the) particle(s), a (the) compound(s), a (the) nanoparticle(s), a(the) bond(s) or a (the) ingredient(s) is/are or represent(s) less than or an assembly of less than 10¹⁰⁰, 10⁵⁰, 10¹⁰, 10⁵, 10³, 10, 5, 2 or 1 particle(s), compound(s), nanoparticle(s), a(the) bond(s) or ingredient(s), preferentially per nm³, mm³, cm³, or m³ preferentially of body part.

In another embodiment of the invention, a (the) particle(s), a (the) compound(s), a (the) nanoparticle(s), a(the) bond(s) or a (the) ingredient(s) is/are or represent(s) between 1 and 10¹⁰⁰, between 1 and 10⁵⁰, between 1 and 10²⁰, between 1 and 10¹⁰, between 1 and 10⁵, between 2 and 10¹⁰⁰, or between 10 and 10¹⁰⁰ particle(s), compound(s), nanoparticle(s), bond(s) or ingredient(s), preferentially per nm³, mm³, cm³, or m³ preferentially of body part.

In still another embodiment of the invention, a nm³, mm³, cm³, or mm³ is a nm³, mm³, cm³, or mm³ of: i) an environment of the particle, ii) an injected region, iii) a body part, iv) a particle region or v) altering medium.

In some cases, a particle region can be a region, volume, surface, length in which the particle is located. The particle can in some cases designate the particle region. In some cases, a particle region can be the region where the nanoparticle is located without the compound or the region where the compound is located without the nanoparticle, e.g. when the compound has diffused towards the infected body part while the nanoparticle has not diffused towards the infected body part and the nanoparticle and compound are therefore located in two different body parts or regions.

In some cases, the particle region can be the volume occupied by an assembly of particles in the body part, where the particles are preferentially: i) separated by less than 10⁹, 10⁶, 10³ or 10 nm, where this distance can be the average distance separating two particles, preferentially measured from the center of external surface of the particles, or ii) at a concentration of more than 10⁻⁵⁰, 10⁻²⁰, 10⁻¹⁰, 10⁻⁵, 10⁻¹, 1, 10, 10³, 10⁵ or 10¹⁰ mg of nanoparticles preferentially per cm³ of body part In some other cases, the particle region can be the volume occupied by an assembly of particles in the body part, where the particles are preferentially: i) separated by more than 10⁻¹, 1, 10³, 10⁶ or 10⁹ nm or ii) at a concentration larger than 10⁵⁰, 10²⁰, 10¹⁰, 10⁵, 10, 1, 10⁻¹, 10⁻³, 10⁻⁵ or 10⁻¹⁰ mg of particles preferentially per cm³ of body part.

In some cases, the particle assembly can designate an assembly of particles before, during, or after particle administration to or in the body part.

In another embodiment of the invention, the body part or particle region has a length, surface area, or volume, which is larger than 10³, 1, 10⁻¹, 10⁻², 10⁻³, 10⁻⁴, 10⁻⁵, 10⁻⁶, 10⁻⁷, 10⁻⁸, 10⁻⁹ or 10⁻²⁰ as measured in m, m², or m³, respectively.

In another embodiment of the invention, the body part, healthy or pathological site, or particle region, has a length, surface area, or volume, which is lower than 10³, 1, 10⁻¹, 10⁻², 10⁻³, 10⁻⁴, 10⁻⁵, 10⁻⁶, 10⁻⁷, 10⁻⁸ or 10⁻⁹ in m, m², or m³, respectively.

In some cases, the environment of the particle, also designated as the environment, can be a liquid, solid or gaseous medium, or length, or surface, or volume, or region, or medium, or at least 1, 10, 10², 10³, 10⁶, 10¹⁰, or 10⁴⁰ substance(s), preferentially at least one substance different from the compound, which surround(s) or include(s) or envelop(s) or comprise(s) the particle over a distance measured from the center or the outer surface of the particle preferably smaller than 10⁵⁰, 10¹⁰, 10⁵, 10³, 10 or 10⁻¹ nm.

In some other cases, the environment of the particle can be a liquid, solid or gaseous medium, or length, or surface, or surface, or volume, or region, or medium, or at least 1, 10, 10², 10³, 10⁶, 10¹⁰, or 10⁴⁰ substance(s), preferentially at least one substance different from the compound, which surround(s) or include(s) or envelop(s) or comprise(s) the particle(s) over a distance measured from the center or the outer surface of the particle preferably larger than 10⁻⁵, 10⁻¹, 1, 5, 10, 10³, 10⁵ or 10¹⁰ nm.

In one embodiment of the invention, the environment of the particle according to the invention comprises: i) the nanoparticle(s) with the compound, ii) the nanoparticle(s) without the compound, or iii) the compound without the nanoparticle(s). In some cases, the bond is in the environment of the particle. In some other cases, the bond is not in the environment of the particle.

In some cases, a substance of the environment of the particle according to the invention may be an atom, a molecule, a polymer, a chemical or biological substance, DNA, RNA, a protein, a lipid, an enzyme, or a nucleic or amino acid.

In some cases, the substance can be different from the compound.

In some other cases, the substance can be the compound.

In another embodiment of the invention, the particles, preferentially the administered particles, are comprised in: i) a suspension or matrix, preferentially before or during their administration to the body part, or ii) the body part, preferentially after or during their administration to the body part.

In another embodiment of the invention, the particles, preferentially the administered nanoparticles, are comprised in a gel, water, a solid, liquid, or gaseous medium or environment or excipient.

In still another embodiment of the invention, the particles are administered at: i) a concentration that is larger than 10⁻²⁰, 10⁻¹⁰, 10⁻¹, 1, 5, 10, 10³, 10⁵ or 10¹⁰ mg of particles per mL or cm³ of suspension or matrix or body part comprising the particles, ii) a speed that is larger than 10⁻²⁰, 10⁻¹⁰, 10⁻¹, 1, 5, 10, 10³, 10⁵ or 10¹⁰ mg of particles per second or hour, or day or month, preferentially per mL or cm³ of suspension or matrix or body part comprising the particles, or iii) using an instrument or equipment such as a syringe, a catheter, needle, needle syringe, patch, perfuser, cannula, endoscope, or dialyser.

In still another embodiment of the invention, the particles are administered at: i) a concentration that is lower than 10²⁰, 10¹⁰, 10, 10⁻¹, 10⁻⁵ or 10⁻¹⁰ mg of nanoparticles per mL or cm³ of suspension or matrix or body part comprising the nanoparticles, ii) a speed that is lower than 10²⁰, 10¹⁰, 10, 10⁻¹, 10⁻⁵ or 10⁻¹⁰ mg of particles per second or hour, or day or month, preferentially per mL or cm³ of suspension or matrix or body part comprising the particles.

In one embodiment of the invention, a property of the initial particle is preferentially measured or exists before or without the alteration.

In another embodiment of the invention, a property of the altered particle is preferentially measured or exists during, after or with the alteration.

In some cases, the property of the particle, preferentially particle size or coating thickness, can be measured by microscopy, optical or electron microscopy, an illuminating technique such as light scattering technique, or another imaging technique such as MRI, scanner, PET-Scan.

In some cases, the size of the particle can be: i) the size of the particle or assembly comprising the nanoparticle and the compound bound to the nanoparticle, ii) the size of the particle or assembly comprising the nanoparticle and the compound released from the nanoparticle, iii) the size of the particle or assembly comprising the nanoparticle and the compound bound to the nanoparticle and the compound released from the nanoparticle, iv) the size of the nanoparticle without the compound bound to the nanoparticle, iv) the size of the nanoparticle without the compound released from the nanoparticle, or v) the size of the nanoparticle without the compound bound to the nanoparticle and without the compound released from the nanoparticle.

In some cases, the size of the particle is not unique, and there preferentially exists a distribution in different particle sizes. Such distribution can be an assembly of particles of different sizes. Such distribution can be represented by plotting the frequency of particle size as a function of the different particle sizes. Such distribution can have a width or full width half maximum (FWHM).

In some cases, the distribution in sizes of the particle is large. This can correspond to large values of the FWHM of the distribution, preferentially larger than 1, 5, 10 or 10³ nm.

In some other cases, the distribution in sizes of the particle is low. This can correspond to small values of the FWHM of the distribution, preferentially smaller than 10⁵, 10³, 100, 70, 50, 20, 10, 5, 2 or 1 nm.

In some cases, the size of the particle can be unique.

In some cases, the size of the particle can be the minimum, average, maximum particle size, preferentially within the distribution in different particle sizes.

In some cases, the size of the particle is the most frequent one, preferentially within the particle size distribution. The most frequent size can be the size that results or yields the maximum size occurrence or size frequency in the particle size distribution.

In some cases, the size of the particle is the size that results in or yields or produces or is associated with the dominant peak or the maximum of the dominant peak within the particle size distribution.

In some cases, the particle size distribution can be characterized by the presence of at least 1, 2, 5 or 10 peaks, where each peak has preferentially a center that is preferentially the average size associated to this peak and preferentially leads to the highest frequency or occurrence of the size within this peak.

In some cases, each peak can be associated with a different mode.

In still some other cases, the particle size distribution can be characterized by the presence of less than 10¹⁰⁰, 10¹⁰, 10, 5, 2 or 1 peak(s).

In some cases, at least two particles can be organized in chains, when the at least two nanoparticles are: i) bound together by some binding material, ii) close to each other, preferentially separated by less than 10⁵, 10³, 10, 5, 2 or 1 nm, or iii) in interaction or bound with each other.

In some other cases, at least two particles are not organized in chains when the at least two nanoparticles are: i) not bound together by some binding material, ii) far from each other, preferentially separated by more than 10⁻⁵, 10⁻¹, 1, 5, 10, 10² or 10⁵ nm, or iii) in interaction with each other.

In some cases, at least two particles are organized in aggregates when they are close to each other, preferentially separated by a distance of less than 10⁵, 10³, 100, 50, 20, 10, 5 or 1 nm, or when they are in interactions with each other.

In some other cases, at least two particles are not organized in aggregates when they are not close to each other, preferentially separated by a distance of more than 10⁻², 10⁻¹, 1, 5, 10, 10³ or 10⁵ nm, or when they are not in interactions with each other.

In some cases, an aggregate can be an assembly of at least two chains of particles.

In some cases, at least two particles can be organized in a geometric figure.

In some cases, a geometric figure is selected from the group consisting of: a Balbis, Concave polygon, Constructible polygon, Convex polygon, Cyclic polygon, Equiangular polygon, Equilateral polygon, Penrose tile, Polyform, Regular polygon, Simple polygon, Tangential polygon, Polygons with specific numbers of sides, Henagon, Digon, Triangle, Acute triangle, Equilateral triangle, Heptagonal triangle, Isosceles triangle, Obtuse triangle, Rational triangle, Right triangle, Kepler triangle, Scalene triangle, Quadrilateral, Cyclic quadrilateral, Kite, Parallelogram, Rhombus, Lozenge, Rhomboid, Rectangle, Square, Tangential quadrilateral, Trapezoid, Isosceles trapezoid, Pentagon, Hexagon, Lemoine hexagon, Heptagon, Octagon, Nonagon, Decagon, Hendecagon, Dodecagon, Tridecagon, Tetradecagon, Pentadecagon, Hexadecagon, Heptadecagon, Octadecagon, Enneadecagon, Icosagon, Swastika, Star polygon, Pentagram—star polygon, Hexagram, Star of David, Heptagram, Octagram, Star of Lakshmi, Decagram—star polygon, Annulus, Arbelos, Circle, Archimedes' twin circles, Bankoff circle, Circumcircle, Disc, Incircle and excircles of a triangle, Nine-point circle, Circular sector, Circular segment, Crescent, Indalo, Lens, Lune, Reuleaux polygon, Reuleaux triangle, Salinon, Semicircle, Tomahawk, Triquetra, Heart, Archimedean spiral, Astroid, Cardioid, Deltoid, Ellipse, Heart, Heartagon, Various lemniscates, Oval, Cartesian oval, Cassini oval, Oval of Booth, Ovoid, Superellipse, Taijitu, Tomoe, and/or Magatama.

In some other cases, the at least two particles are not organized in a geometric figure.

In one embodiment of the invention, the distribution of particles is homogenous. In this case, the distribution is size, volume, mass of the particle can be lower than 100, 90, 70, 60, 50, 40, 30, 20, 10, 5, 2 or 1%. In some cases, this percentage can be equal to Tmax−Tmin/Tmax, Tmin/Tmax, Vmax−Vmin/Vmax, Vmin/Vmax Mmax−Mmin/Mmax, Mmin/Mmax, where Tmax, Tmin, Vmax, Vmin, Mmax, Mmin are the maximum size, minimum size, maximum volume, minimum volume, maximum mass, and minimum mass, within the particle distribution in size, volume, and mass.

In another embodiment of the invention, the distribution of particles is non-homogenous. In this case, the distribution in size, volume, mass of the particle can be larger than 10⁻⁵⁰, 10⁻¹⁰, 10⁻¹, 1, 5, 10, 20, 50, 70, 90 or 99%.

As described in the experimental section, the magnetosomes can be arranged in chains before alteration and not be arranged in chains following alteration.

In some cases, a physico-chemical disturbance can be applied on said altered particle, preferentially during step 7) of the method.

In some cases, the altered particle can have a size that is smaller than the size of the initial particle, by a percentage preferentially between 10⁻³% and 99.99%, where this percentage is preferentially S_{A}/S_{I} or (S_{I}−S_{A})/S_{I}, where S_{A} and S_{I} are the sizes of the altered and initial particles, respectively.

In some cases, the particle size can decrease from the size of the initial particle down to the size of the altered particle.

In some cases, the altered particle can have a size that is smaller than the size of the initial particle, preferentially when the particle size can increase from the size of the initial particle up to the size of the altered particle.

In some cases, the altered particle can have a number of altered compounds bound to the altered nanoparticle, nₐ, that is smaller than the number of compounds bound to the initial nanoparticle, nᵢ, where nᵢ/nₐ is preferentially between 1 and 10¹⁰, preferentially when the number of compounds bound to the nanoparticle decreases, preferentially from a number nᵢ of initial compounds bound to the initial nanoparticle down to a number nₐ of altered compounds bound to the altered nanoparticle.

In some other cases, the altered particle can have a number of altered compounds bound to the altered nanoparticle, nₐ, that is larger than the number of compounds bound to the initial nanoparticle, nᵢ, preferentially when the number of compounds bound to the nanoparticle increases, preferentially from a number nᵢ of initial compounds bound to the initial nanoparticle down to a number nₐ of altered compounds bound to the altered nanoparticle.

In some cases, the altered particle can have a binding strength of least one bond between the altered compound and the altered nanoparticle, Sₐ, that is smaller than the binding strength of at least one bond between the initial compound and the initial nanoparticle, Sᵢ, preferentially when the binding strength of least one bond between the compound and the nanoparticle decreases, from a binding strength Sᵢ of at least one bond between the initial compound and the initial nanoparticle to a binding strength Sₐ of at least one bond between the altered compound and the altered nanoparticle.

In some cases, the altered particle can have a binding strength of least one bond between the altered compound and the altered nanoparticle, Sₐ, that is larger than the binding strength of at least one bond between the initial compound and the initial nanoparticle, Sᵢ, preferentially when the binding strength of least one bond between the compound and the nanoparticle increases, from a binding strength Sᵢ of at least one bond between the initial compound and the initial nanoparticle up to a binding strength Sₐ of at least one bond between the altered compound and the altered nanoparticle.

In some cases, the altered particle can have or be subjected to the breaking or weakening of at least one bond between the altered compound and the altered nanoparticle.

In some other cases, the altered particle can have or be subjected to an absence of breaking or weakening of at least one bond between the altered compound and the altered nanoparticle.

In some cases, the altered particle can have a bond-dissociation energy between the altered compound and the altered nanoparticle, E_{da}, that is smaller than the bond-dissociation energy between the initial compound and the initial nanoparticle, E_{di}, preferentially when the bond-dissociation energy between the compound and the nanoparticle decreases, from a bond-dissociation energy E_{di} between the initial compound and the initial nanoparticle down to a bond-dissociation energy E_{da} between the altered compound and the altered nanoparticle.

In some other cases, the altered particle can have a bond-dissociation energy between the altered compound and the altered nanoparticle, E_{da}, that is larger than the bond-dissociation energy between the initial compound and the initial nanoparticle, E_{di}, preferentially when the bond-dissociation energy between the compound and the nanoparticle increases, from a bond-dissociation energy E_{di} between the initial compound and the initial nanoparticle up to a bond-dissociation energy E_{da} between the altered compound and the altered nanoparticle.

In some cases, the altered particle can have a coating thickness, CTₐ, that is smaller than the coating thickness of the initial nanoparticle, CTᵢ, preferentially when the coating thickness of the nanoparticle decreases, from a coating thickness CTᵢ of the initial nanoparticle down to a coating thickness CTₐ of the altered nanoparticle.

In some other cases, the altered particle can have a coating thickness, CTₐ, that is larger than the coating thickness of the initial nanoparticle, CTᵢ, preferentially when the coating thickness of the nanoparticle increases, from a coating thickness CTᵢ of the initial nanoparticle up to a coating thickness CTₐ of the altered nanoparticle.

In some cases, the altered particle can have a percentage in mass of organic material or carbon or carbonaceous material of the altered particle that is smaller than the percentage in mass of organic material or carbon or carbonaceous material of the initial particle, preferentially when the percentage in mass of organic material or carbon or carbonaceous material of the altered particle has decreased, compared with the percentage in mass of organic material or carbon or carbonaceous material of the initial particle.

In some other cases, the altered particle can have a percentage in mass of organic material or carbon or carbonaceous material of the altered particle that is larger than the percentage in mass of organic material or carbon or carbonaceous material of the initial particle, preferentially when the percentage in mass of organic material or carbon or carbonaceous material of the altered particle has increased, compared with the percentage in mass of organic material or carbon or carbonaceous material of the initial particle.

In some cases, the altered particle can have or be prone to a cluttering of the altered compound bound to the altered nanoparticle that is smaller than the cluttering of the initial compound bound to the initial nanoparticle, preferentially when the cluttering of the compound bound to the nanoparticle decreases, from a large cluttering of the initial compound bound to the initial nanoparticle down to a small cluttering of the altered compound bound to the altered nanoparticle.

In some cases, the altered particle can have or be prone to a cluttering of the altered compound bound to the altered nanoparticle that is larger than the cluttering of the initial compound bound to the initial nanoparticle, preferentially when the cluttering of the compound bound to the nanoparticle increases, from a small cluttering of the initial compound bound to the initial nanoparticle up to a large cluttering of the altered compound bound to the altered nanoparticle.

In some cases, the altered particle can have a number of altered compounds N₁ₐ that prevent the release of altered compounds N₂ₐ from the altered nanoparticle that is smaller than the number of initial compounds N₁ᵢ that prevent the release of initial compounds N₂ᵢ from the initial nanoparticle, preferentially when the number or concentration of compounds N₁ that prevent the release of compounds N₂ from the nanoparticle decreases, from a number of initial compounds N₁ᵢ that prevent the release of initial compounds N₂ᵢ from the initial nanoparticle down to a number of altered compounds N₁ₐ that prevent the release of altered compounds N₂ₐ from the altered nanoparticle.

In some other cases, the altered particle can have a number of altered compounds N₁ₐ that prevent the release of altered compounds N₂ₐ from the altered nanoparticle that is larger than the number of initial compounds N₁ᵢ that prevent the release of initial compounds N₂ᵢ from the initial nanoparticle, preferentially when the number or concentration of compounds N₁ that prevent the release of compounds N₂ from the nanoparticle increases, from a number of initial compounds N₁ᵢ that prevent the release of initial compounds N₂ᵢ from the initial nanoparticle up to a number of altered compounds N₁ₐ that prevent the release of altered compounds N₂ₐ from the altered nanoparticle.

In one embodiment of the invention, the physico-chemical disturbance is or results in or is associated with a decrease or increase in particle size or particle size distribution, preferentially from the size of the altered particle SA down to or up to the size of the altered and disturbed particle SAD, which is: i) equal, ii) none or smaller or iii) larger to/than the decrease in size of the particle due to alteration.

In one embodiment of the invention, the physico-chemical disturbance is or results in or is associated with a decrease of the number of compounds attached or bound to the nanoparticle, preferentially in the following manner: i) by a factor of at least 1.001, 1.1, 1.5, 2, 5, 10, 10³, 10⁵, or 10¹⁰, ii) from more than 1, 5, 10, 10³, 10⁵, 10¹⁰ or 10⁵⁰ altered compound(s), preferentially per altered nanoparticle, attached or bound to the altered nanoparticle before physico-chemical disturbance to less than 10⁵⁰, 10¹⁰, 10⁵, 10³, 10, 5 or 1 altered and disturbed compound(s), preferentially per altered and disturbed nanoparticle, preferentially attached or bound to the altered and disturbed nanoparticle during or after physico-chemical disturbance, or iii) by a none, equal, smaller or larger amount preferentially compared with the decrease in number of compounds due to alteration.

In one embodiment of the invention, the physico-chemical disturbance is or results in or is associated with a decrease or increase of the strength of at least one bond between the compound and the nanoparticle, preferentially from a strength Sa of at least one altered bond between the altered compound and the altered nanoparticle to a strength Sad of at least one altered and disturbed bond between the altered and disturbed compound and the altered and disturbed nanoparticle, where this increase or decrease is preferentially equal, larger or smaller to or than the decrease or increase of the strength of the bond between the initial bond and the initial nanoparticle.

In one embodiment of the invention, the physico-chemical is or results in or is associated with a decrease or increase of the bond-dissociation energy between the compound and the nanoparticle, preferentially from a bond-dissociation energy E_{da} between the altered compound and the altered nanoparticle down to or up to a bond-dissociation energy E_{dad} between the altered and disturbed compound and the altered and disturbed nanoparticle.

In some cases, E_{da}, E_{dad}, E_{da}/E_{dad} can be equal to, larger than, preferentially by a factor of at least 0, 0.5, 1, 1.1, 1.5, 2, 5, 10, 10², 10³ or 10⁵, or smaller than, preferentially by a factor of at least 0, 0.5, 1, 1.1, 1.5, 2, 5, 10, 10², 10³ or 10⁵, E_{di}, E_{da}, E_{di}/E_{da}, as previously defined.

In still another embodiment of the invention, the physico-chemical disturbance is or results in or is associated with a decrease or increase of the thickness of the coating of said nanoparticle, preferentially by the same quantity as or a larger quantity than or a smaller amount than the decrease or increase of the thickness of the coating of said nanoparticle due to alteration.

In one embodiment of the invention, the physico-chemical is or results in or is associated with a decrease or increase of the cluttering of the compound bound to the nanoparticle, which is equal, smaller or larger than the decrease or increase of the cluttering of the compound due to alteration.

In one embodiment of the invention, the physico-chemical disturbance is or results in or is associated with a modification of the chemical composition of the altered particle, also designated as second chemical modification, which is the same, similar or different from the first chemical modification due to alteration.

In some cases, the modification of at least one property of the altered particle due to physico-chemical disturbance when it transforms from the altered to the altered and disturbed particle can be the same as the modification of at least one property of the initial particle due to alteration when it transforms from the initial to the altered particle.

In some cases, i) the variation, increase or decrease of the percentage in mass of organic material or carbon or carbonaceous material of the particle, ii) the variation, decrease or increase of the mass or weight of the particle, iii) the modification of at least one magnetic property of the particle, iv) the variation, decrease or increase of at least one magnetic parameter of the particle, v) the modification of a property of assembly, organization, and/or distribution of the nanoparticle, vi) the modification of the crystallinity of the particle, vii) the modification of the morphology or geometry or of particle, viii) the modification of the number of facets or edges or corners of the particle, ix) the variation of the SAR of the particle, and/or x) the variation of the quantity of radical or reactive species produced by the particle, can be the same, similar, larger or smaller during physico-chemical disturbance than during alteration.

In one embodiment of the invention, the altered particle is the particle that has undergone alteration, or has been exposed or subjected to alteration. Preferentially before alteration, the particle is non- or not altered. Preferentially, during or after alteration, the particle is altered.

In some cases, the alteration can be the alteration of the particle, nanoparticle, compound, and/or bond between the nanoparticle and compound.

In some cases, the compound can be the initial compound, the altered compound, or the altered and disturbed compound.

In some cases, the compound as defined in the invention is activated when it is released or detached from the nanoparticle.

In some other cases, the compound as defined in the invention is not activated when it is not released or not detached from the nanoparticle or when it is bound to the nanoparticle.

In some other cases, the compound as defined in the invention is activated when the compounds triggers at least one of the following events or at least one of the following events occurs: i) a virus is inactivated or attenuated or destroyed, ii) antigens are activated, preferentially presented to or in interaction with immune cells such as B cells, T cells, antigen presenting cells, or complexes such as MHC, and iii) antibodies are produced.

In some other cases, the compound is not activated when at least one of the events mentioned in the previous embodiment does not occur.

In some cases, the quantity or number of compounds released from the nanoparticle is larger by a factor of at least 0, 1, 1.1, 2, 5, 10, 10³, 10⁵ or 10¹⁰ when the method or at least one step of the method is used or is followed than when the method or at least one step of the method is not used or is not followed.

In one embodiment of the invention, the altered compound is the compound that has at least one property resulting from, originating from, or produced by the alteration.

In some cases, when the compound is a virus, the alteration can result in the inactivation or attenuation of the virus, preferentially following interactions, binding of the virus with the nanoparticle or internalization of the nanoparticle in the virus, preferentially following the application of a physico-chemical disturbance, such as a change in pH or temperature, on the particle, and/or preferentially following the application of a radiation on the nanoparticle.

In an embodiment of the invention, the compound is selected from the group consisting of: i) the initial compound, i.e. the compound that is preferentially not exposed to alteration and/or physico-chemical disturbance or that is preferentially not released by alteration and/or physico-chemical disturbance from the initial nanoparticle, ii) the altered compound, i.e. the compound that is preferentially exposed to the alteration and is preferentially releasable or released by alteration from the altered nanoparticle, and iii) the altered and disturbed compound, i.e. the compound that is preferentially exposed to alteration and physico-chemical disturbance and is preferentially releasable or released by alteration and physico-chemical disturbance from the altered and disturbed nanoparticle.

In some cases, the releasable initial compound can be the same as the releasable altered compound.

In some cases, a first partial release can generate the release of a first part of altered compound from the altered nanoparticle during or following the alteration.

In some cases, an absence of release can generate a second part of altered compounds that remain bound to the altered nanoparticle during or after alteration.

In some cases, a second partial release can generate the release of group 2 of second part of altered and disturbed compounds from the altered and disturbed nanoparticle during or after physico-chemical disturbance.

In some other cases, an absence of release, preferentially following the physico-chemical disturbance, can generate the group 1 of second part of altered and disturbed compounds comprising altered and disturbed compounds bound to the altered and disturbed nanoparticle, preferentially following, during or with physico-chemical disturbance.

The invention also relates to the method or compound according to the invention, wherein the compound is selected in the group consisting of: i) at least one ion, ii) at least one atom, preferentially metallic, preferentially iron, iii) at least one molecule, iv) at least one nanoparticle, v) at least one radical specie, vi) a contrast agent, vii) a luminescent compound, viii) a drug or medicament, ix) a medical device, x) a cosmetic compound, xi) a therapeutic compound, xii) a medical compound, xiii) a biological compound, xiv) a diagnostic compound, xv) a medical equipment or apparatus, xvi) a composition, xvii) a suspension, xviii) an excipient, xix) an adjuvant, xx) a cytotoxic compound, xxi) a non-cytotoxic compound, xxii) an immunogenic compound, xxiii) a non-immunogenic compound, xxiv) a pharmacological compound, xxv) a non-pharmacological compound, xxvi) a metabolic compound, xxvii) a non-metabolic compound, xxviii) an antigen or an epitope, xxix) an antibody, xxx) a vaccine, xxxi) a virus, preferentially an attenuated or inactivated virus, xxxii) a metal, preferentially a non-toxic metal, iron, silver, or gold, xxxiii) an antibiotic, xxxiv) a bacterium, xxxv) an antiviral agent, xxxvi) a compound released from the nanoparticle, xxxvii) a compound bound to the nanoparticle, xxxviii) an inactivated compound, xxxix) an activated compound, and xxxx) a derivative or part or portion of any of these compounds.

In some cases, the nanoparticle or particle can be or share a property with at least one compound preferentially listed in the previous embodiment.

In one embodiment of the invention, the compound is bound to the nanoparticle or is not released from the nanoparticle, preferentially before, during, or after step a), b), a), and/or P) of the method. In this case, the compound can be bound to the nanoparticle or not released from the nanoparticle in the following situation(s): i) before the particle is exposed to the alteration or when the particle is not exposed to the alteration or before the alteration of the particle or when the alteration of the particle does not occur, or ii) when the compound belongs to the second part of the compound, where the second part of the compound is preferentially the quantity or number of the compound that remains bound to the nanoparticle after alteration.

In another embodiment of the invention, the compound is not bound to the nanoparticle or is released from the nanoparticle, preferentially before, during, or after step a), a), and/or b), P) of the method. In this case, the compound can be unbound from the nanoparticle or released from the nanoparticle in the following situations: i) after or during the exposition of the particle to the alteration or after or during the alteration of the particle or ii) when the compound belongs to the first part of the compound, where the first part of the compound is preferentially the quantity or number of the compound that is released from the nanoparticle after or during alteration.

In some cases, the compound can be or be comprised in a composition.

In an embodiment of the invention, the nanoparticle is selected from the group consisting of: i) the initial nanoparticle, i.e. the nanoparticle that is preferentially bound to the initial compound and is preferentially not exposed to the alteration and the physico-chemical disturbance, ii) the altered nanoparticle, i.e. the nanoparticle that can preferentially be separated, dissociated, or released from the altered compound by alteration, and is preferentially exposed to alteration, and iii) the altered and disturbed nanoparticle, i.e. the nanoparticle that can preferentially be separated, dissociated, or released from the altered and disturbed compound by alteration and physico-chemical disturbance and that is preferentially exposed to alteration and physico-chemical disturbance.

In one embodiment, the nanoparticle can be made of a central part that is covered, surrounded, or enveloped, partly or fully, by a coating.

In still some other cases, the organic material or carbon is comprised in the central part and/or coating of the nanoparticle, preferentially predominantly in the coating.

In some cases, the bond is attached to or comprised in the coating and/or central part.

In some other cases, the compound is attached to or comprised in the coating and/or central part.

In one embodiment of the invention, the altered nanoparticle is the nanoparticle that has at least one property resulting from, originating from, or produced by the alteration.

In some cases, the injected region can be the region in which the particles are injected or administered or located, preferentially in or to the body part. It can be designated as particle region. The particles can be injected/administered to/in the injected region.

The invention also relates to the method according to the invention, wherein the nanoparticles have at least one property selected from the group consisting of: i) a size in the range from 1 to 10³ nm, and ii) a metallic, magnetic, and/or crystallized structure or composition.

In one embodiment of the invention, the nanoparticle(s) is/are the nanoparticle(s) treated by the method according to the invention, preferentially altered and/or disturbed.

In one embodiment of this invention, the nanoparticle(s) is/are selected from the group of nanoparticle(s) consisting of: i) a nanosphere, ii) a nanocapsule, iii) a dendrimer, iv) a carbon nanotube, v) a lipid/solid nanoparticle, vi) a lipid or protein or DNA or RNA based nanoparticle, vii) a nanoparticle with an inner aqueous environment or with an inner solid core, which is preferentially crystallized, which is preferentially surrounded by a layer, preferentially a stabilizing layer, most preferentially a phospholipid layer, viii) a multilayer nanoparticle, ix) a polymeric nanoparticle, x) a quantum dot, xi) a metallic nanoparticle, xii) a polymeric micelle or nanoparticle, xiii) a carbon based nano-structure, xiv) a nanobubble, xv) a nanosome, xvi) a pharmacyte, xvii) a niosome, xviii) a nanopore, xix) a microbivore, xx) a liposome, xxi) a virus, preferentially recombinant, xxii) a herbal nanoparticle, xxiii) an antibody, and/or xxiv) a vesicle.

In another embodiment of this invention, the nanoparticle is not: i) a nanosphere, ii) a nanocapsule, iii) a dendrimer, iv) a carbon nanotube, v) a lipid/solid nanoparticle, vi) a lipid or protein or DNA or RNA based nanoparticle, vii) a nanoparticle with an inner aqueous environment or with an inner solid core, which is preferentially crystallized, which is preferentially surrounded by a layer, preferentially a stabilizing layer, most preferentially a phospholipid layer, viii) a multilayer nanoparticle, ix) a polymeric nanoparticle, x) a quantum dot, xi) a metallic nanoparticle, xii) a polymeric micelle or nanoparticle, xiii) a carbon based nano-structure, xiv) a nano-bubble, xv) a nanosome, xvi) a pharmacyte, xvii) a niosome, xviii) a nanopore, xix) a microbivore, xx) a liposome, xxi) a virus, preferentially recombinant, xxii) a herbal nanoparticle, xxiii) an antibody, and/or xxiv) a vesicle.

In some cases, the nanoparticles can be in a liquid, gaseous, or solid state, preferentially before, during or after its presence or administration in/to the body part.

In some other cases, the nanoparticles can't be in one or two of the liquid, gaseous, or solid states, preferentially before, during or after its presence or administration in/to the body part.

In still some other cases, the nanoparticles can be assimilated to or be comprised in a ferrofluid, a chemical or biological ferrofluid, wherein chemical and biological ferrofluids are fluids containing iron, preferentially forming nanoparticles, which are fabricated through a chemical or biological synthesis, respectively.

In still some other cases, the ferrofluid or nanoparticles assembly can comprise the nanoparticles and an excipient, a solvent, a matrix, a gel, which preferentially enables the administration of the nanoparticles to the individual or body part.

In still some other cases, the nanoparticles can comprise synthetic material and/or biological material and/or inorganic material and/or organic material.

In one embodiment of the invention, (the) nanoparticle(s) is/are or designate(s): i) a suspension of nanoparticles, ii) a composition comprising nanoparticles, iii) an assembly of nanoparticles, iv) a nanoparticle region, preferentially a volume or region comprising at least one nanoparticle, v) the mineral, central, core, or crystallized part of the nanoparticle(s), vi) the organic part of the nanoparticle(s), vii) the inorganic part of the nanoparticle(s), or viii) the coating of the nanoparticle(s), where the coating is preferentially a layer, material, or part of the nanoparticle(s), which envelops, surrounds, protects, or stabilizes the mineral, central, core, or crystallized part of the nanoparticle(s).

In one embodiment of the invention, (the) nanoparticles(s) represent(s) or is or are an assembly or suspension or composition of more or comprising more than 10⁻¹⁰⁰, 10⁻⁵⁰, 10⁻¹⁰, 10⁻⁵, 10⁻¹, 1, 10, 10², 10³, 10⁵, 10¹⁰, 10²⁰ or 10⁵⁰ nanoparticles(s) or mg of nanoparticles(s) or mg of metallic element or iron comprised in nanoparticles(s) or mg of nanoparticles(s) per cm³ or mg of nanoparticles(s) per cm³ of body part or mg of iron or metallic element comprised in nanoparticles(s) per cm³ or mg of iron or metallic element comprised in nanoparticles(s) per cm³ of body part. In some cases, an assembly or suspension or composition comprising a large number of nanoparticles can be used to induce or produce: i) a temperature increase, ii) radical or reactive species, or iii) the dissociation of a compound from the nanoparticles.

In another embodiment of the invention, (the) nanoparticle(s) represent(s) or is or are an assembly or suspension or composition of less or comprising less than 10¹⁰⁰, 10⁵⁰, 10²⁰, 10¹⁰, 10⁵, 10², 10, 1, 5, 2, 1, 10⁻¹, 10⁻⁵, 10⁻¹⁰ or 10⁻⁵⁰ nanoparticle(s) or mg of nanoparticles or mg of iron or metallic element comprised in nanoparticles or mg of nanoparticle per cm³ or mg of nanoparticles per cm³ of body part or mg of iron comprised in nanoparticles(s) per cm³ or mg of iron or metallic element comprised in nanoparticles(s) per cm³ of body part. In some cases, an assembly or suspension or composition of nanoparticles comprising a low number of nanoparticles(s) can be used to prevent toxicity.

In one embodiment of the invention, (the) nanoparticles(s) or nanoparticle assembly is the region, also designated as nanoparticles region, volume, surface, length, which comprises the nanoparticles or where nanoparticles are located. In some cases, the volume of the region occupied by the nanoparticles in the body part is designated as nanoparticles region.

In some cases, the nanoparticle region can be the volume occupied by an assembly of nanoparticles in the body part, where the nanoparticles are preferentially: i) separated by less than 10⁹, 10⁶, 10³ or 10 nm, where this distance can be the average distance separating two nanoparticles, preferentially measured from the center of external surface of the nanoparticles, or ii) at a concentration of more than 10⁻⁵, 10⁻²⁰, 10⁻¹⁰, 10⁻¹, 10⁻¹, 1, 10, 10³, 10⁵ or 10¹⁰ mg of nanoparticles per cm³ of body part In some other cases, the nanoparticle region can be the volume occupied by an assembly of nanoparticles in the body part, where the nanoparticles are preferentially: i) separated by more than 10⁻¹, 1, 10³, 10⁶ or 10⁹ nm or ii) at a concentration larger than 10⁵⁰, 10²⁰, 10¹⁰, 10⁵, 10, 1, 10⁻¹, 10⁻³, 10-s or 10⁻¹⁰ mg of nanoparticles per cm³ of body part.

In some cases, the nanoparticle assembly can designate an assembly of nanoparticles before, during, or after nanoparticle administration to or in the body part.

The invention also relates to nanoparticles for use according to the invention, wherein the nanoparticles are crystallized, metallic, or magnetic.

In an embodiment of the invention, the nanoparticles are crystallized. In this case, they preferentially possess more than or at least 1, 2, 10, 10², 10³, 10⁶ or 10⁹ crystallographic plane(s) or regular atomic arrangement(s), preferentially observable by electron microscopy.

In one embodiment of the invention, the nanoparticles are metallic. In this case, they contain at least 1, 10, 10³, 10⁵ or 10⁹ metallic atom(s) or contain at least 1, 10, 50, 75 or 90% of metallic atoms, where this percentage can be the ratio between the number or mass of metallic atoms in the nanoparticles divided by the total number or mass of all atoms in the nanoparticles. In some cases, the nanoparticles, preferentially metal oxide nanoparticles, can also contain at least 1, 10, 10³, 10⁵ or 10⁹ oxygen atom(s), or contain at least 1, 10, 50, 75 or 90% of oxygen atoms, where this percentage can be the ratio between the number or mass of oxygen atoms in the nanoparticles divided by the total number or mass of all atoms in the nanoparticles.

In some cases, an atom can be a chemical element or an element.

In another embodiment of the invention, the metal or metal atom is selected in the list consisting of: Lithium, Beryllium, Sodium, Magnesium, Aluminum, Potassium, Calcium, Scandium, Titanium, Vanadium, Chromium, Manganese, Iron, Cobalt, NIckel, Copper, Zinc, Gallium, Rubidium, Strontium, Yttrium, Zirconium, Niobium, Molybdenum, Technetium, Ruthenium, Rhodium, Palladium, Silver, Cadmium, Indium, Tin, Cesium, Barium, Lanthanum, Cerium, Praseodymium, Neodymium, Promethium, Samarium, Europium, Gadolinium, Terbium, Dysprosium, Holmium, Erbium, Thulium, Ytterbium, Lutetium, Hafnium, Tantalum, Tungsten, Rhenium, Osmium, Iridium, Platinum, Gold, Mercury, Thallium, Lead, Bismuth, Polonium, Francium, Radium, Actinium, Thorium, Protactinium, Uranium, Neptunium, Plutonium, Americium, Curium, Berkelium, Californium, Einsteinium, Fermium, Mendelevium, Nobelium, Lawrencium, Rutherfordium, Dubnium, Seaborgium, Bohrium, Hassium, Meitnerium, Darmstadtium, Roentgenium, Copernicium, Nihonium, Flerovium, Moscovium, and Livermorium or Livermorium atom.

In another embodiment of the invention, the nanoparticles contain less than 1, 10, 10³, 10⁵ or 10⁹ metallic atom(s) or contains less than 1, 10, 50, 75 or 90% of metallic atoms, where this percentage can be the ratio between the number or mass of metallic atoms in the nanoparticles divided by the total number or mass of all atoms in the nanoparticles. It can also contain less than 1, 10, 10³, 10⁵ or 10⁹ oxygen atom(s), or contain less than 1, 10, 50, 75 or 90% of oxygen atoms, where this percentage can be the ratio between the number or mass of oxygen atoms in the nanoparticles divided by the total number or mass of all atoms in the nanoparticles.

In one embodiment of the invention, the nanoparticle is magnetic when it has a magnetic behavior or property, where the magnetic behavior or property is preferentially selected from the group consisting of a diamagnetic, superparamagnetic, paramagnetic, ferromagnetic, and ferrimagnetic behavior or property.

In some cases, the magnetic behavior or property can be observed or exist at a temperature, which is lower than: i) 10⁵, 10³, 500, 350, 200, 100, 50, 20, 10, 1, 0.5 or 1 K (Kelvin), ii) the Curie temperature, iii) the melting or fusion temperature, or iv) the blocking temperature.

In some other cases, the magnetic behavior or property can be observed or exist at a temperature, which is larger than: i) 0.5, 1, 10, 20, 50, 100, 200, 350, 500, 10³ or 10⁵ K, ii) the Curie temperature, iii) the melting temperature or iv) the blocking temperature.

In still some other cases, the magnetic behavior or property can be observed or exist at a temperature, which is between 10⁻²⁰ and 10²⁰ K, or between 0.1 and 1000 K.

In one embodiment of the invention, the nanoparticles have or are characterized by at least one property selected from the group consisting of: i) the presence of a core, preferentially magnetic, preferentially mineral, preferentially composed of a metallic oxide such as iron oxide, most preferentially maghemite or magnetite, or an intermediate composition between maghemite and magnetite, ii) the presence of a coating that surrounds the core and preferentially prevents nanoparticles aggregation, preferentially enabling nanoparticles administration in an organism or in the body part or stabilizing the nanoparticles core, where coating thickness may preferably lie between 0.1 nm and 10 μm, between 0.1 nm and 1 μm, between 0.1 nm and 100 nm, between 0.1 nm and 10 nm, or between 1 nm and 5 nm, iii) magnetic properties leading to diamagnetic, paramagnetic, superparamagnetic, ferromagnetic, or ferrimagnetic behavior, iv) a coercivity larger than 0.01, 0.1, 1, 10, 100, 10³, 10⁴, 10⁵, 10⁹ or 10²⁰ Oe, v) a ratio between remanent and saturating magnetization larger than 0.01, 0.1, 0.2, 0.3, 0.4, 0.5, 0.75, 0.9 or 0.99, vi) a saturating magnetization larger than 10⁻⁵⁰, 0.1, 1, 5, 10 or 50 emu per gram of nanoparticle, vii) magnetic properties such as coercivity, remanent and saturating magnetization, preferentially measured or observed or existing at a temperature larger than 0.1 K, 1 K, 10 K, 20 K, 50 K, 100 K, 200 K, 300 K, 350 K or 3000 K, viii) a crystallinity, i.e. nanoparticles preferentially possessing at least 1, 2, 5, 10 or 100 crystalline plane(s), preferentially observable or measured by electron microscopy, ix) the presence of a single domain, x) a size that is larger than 0.1, 0.5, 1.5, 10, 15, 20, 25, 30, 50, 60, 70, 80, 100, 120, 150 or 200 nm, xi) a size lying between 0.1 nm and 10 μm, between 0.1 nm and 1 μm, between 0.1 nm and 100 nm, between 1 nm and 100 nm, or between 5 nm and 80 nm, xii) a non-pyrogenicity or apyrogenicity, which preferentially means that nanoparticles possess an endotoxin concentration lower than 10²⁰, 10000, 1000, 100, 50, 10, 5, 2 or 1 EU (endotoxin unit) per mg of nanoparticles or per mg of iron comprised in nanoparticles, or which means that nanoparticles do not trigger fever or an increase in whole body temperature larger than 100, 50, 6.6, 5, 3, 2 or 1° C. following their administration to a living organism or body part, xiii) a synthesis by a synthetizing living organism, preferentially by bacteria, xiv) a chemical synthesis, xv) the presence of less than 50, 25, 15, 10, 5, 2 or 1%, preferentially in mass or volume, of organic or carbon material originating from the synthetizing living organism, xv), the presence of more than 99, 95, 80, 70, 60, 50 or 25%, preferentially in mass or volume, of mineral material originating from the synthetizing living organism, and/or xvi) a specific absorption rate (SAR) that is larger than 1, 10, 1000 or 10⁴ Watt per gram of nanoparticles, preferentially measured under the application of an alternating magnetic field of strength preferentially larger than 0.1, 1, 10 or 100 mT, and/or frequency larger than 1, 10, 100 or 1000 KHz, alternatively preferentially measured under the application of the acoustic wave, alternatively under the application of a radiation such as an electromagnetic acoustic, or light radiation.

In some cases, the synthetizing living organism can be magnetotactic bacteria, bacteria that synthesize nanoparticles inside them, other types of bacteria than magnetotactic bacteria or enzymes of certain bacteria, preferentially synthetizing nanoparticles extra-cellularly, such as Mycobacterium paratuberculosis, Shewanella oneidensi, Geothrix fermentans, ants, fungi, or various plants.

In another embodiment of the invention, the nanoparticles have or are characterized by at least one property selected from the group consisting of: i) a coercivity lower than 0.01, 0.1, 1, 10, 100, 10³, 104, 105, 109 or 10²⁰ Oe, ii) a ratio between remanent and saturating magnetization lower than 0.01, 0.1, 0.2, 0.3, 0.4, 0.5, 0.75, 0.9 or 0.99, iii) a saturating magnetization lower than 0.1, 1, 5, 10, 50, 200, 1000 or 5000 emu/g, iv) magnetic properties preferentially measured or observed at a temperature lower than 0.1 K, 1 K, 10 K, 20 K, 50 K, 100 K, 200 K, 300 K, 350 K or 3000 K, v) a size that is lower than 0.1, 0.5, 1.5, 10, 15, 20, 25, 30, 50, 60, 70, 80, 100, 120, 150 or 200 nm, vi) the presence of more than 50, 25, 15, 10, 5, 2 or 1%, preferentially in mass or volume, of organic or carbon material originating from the synthetizing living organism, vii) the presence of less than 99, 95, 80, 70, 60, 50 or 25%, preferentially in mass or volume, of mineral material originating from the synthetizing living organism, or xi), a specific absorption rate (SAR) that is lower than 1, 10, 1000 or 10⁴ Watt per gram of nanoparticles, preferentially measured under the application of an alternating magnetic field of strength preferentially lower than 0.1, 1, 10, or 100, 200, 500, 10³ or 10⁵ mT, and/or of frequency preferentially lower than 1, 10, 100, 10³, 10⁵ or 10⁹ KHz, alternatively preferentially measured under the application of the acoustic wave, alternatively under the application of a radiation such as an electromagnetic acoustic, or light radiation.

In some cases, the mineral can be the part of the nanoparticles or magnetosome that does not comprise organic material or comprises a low percentage in mass or volume of organic material, preferentially less than 100, 99, 50, 20, 10, 5, 1, 10⁻¹ or 10⁻² percent or percent in mass or volume of organic material. The mineral is preferentially the core of the nanoparticles.

In some other cases, the mineral can comprise a percentage in mass or volume of organic material larger than 0, 10⁻⁵⁰, 10⁻¹⁰, 10⁻², 10⁻¹ or 1 percent or percent in in mass or volume of organic material.

This can be the case when the purification step unsuccessfully removes the organic material or when organic material is added to the mineral after the purification step.

In some cases, the nanoparticles can be surrounded by a coating. The coating can be made of a synthetic, organic, or inorganic material or of a substance comprising a function selected in the group consisting of: carboxylic acids, phosphoric acids, sulfonic acids, esters, amides, ketones, alcohols, phenols, thiols, amines, ether, sulfides, acid anhydrides, acyl halides, amidines, amides, nitriles, hydroperoxides, imines, aldehydes, and peroxides. In some cases, the coating can be made of carboxy-methyl-dextran, citric acid, phosphatidylcholine (DOPC), and/or oleic acid.

In some cases, the coating can enable the dispersion of the nanoparticles in a matrix or solvent such as water, preferentially without aggregation or sedimentation of the nanoparticles.

In some cases, the coating can enable internalization of the nanoparticles in cells.

In some other cases, the coating can enable: i) to bind two or more nanoparticles(s) together preferentially in a chain, ii) to prevent nanoparticles aggregation and/or, iii) to obtain uniform nanoparticles distribution.

In one embodiment of the invention, the nanoparticles are non-pyrogenic. Non-pyrogenic nanoparticles preferentially: i) comprise less than 10¹⁰⁰, 10⁵⁰, 10²⁰, 10⁸, 10⁵, 10³, or 10 EU (endotoxin unit) or EU per cm³ of body part or EU per mg of nanoparticles or EU per cm³ of body part per mg of nanoparticles, or ii) induce a temperature increase of the individual or body part of less than 10⁵, 10³, 10², 50, 10, 5, 4, 3, 2 or 1° C., preferentially above physiological temperature, preferentially before, after or without the application of the acoustic wave or radiation on the nanoparticles.

In one embodiment of this invention, the nanoparticles or compound is composed of or comprises a chemical element of the families selected from the group consisting of: metals (alkali metal, alkaline earth metal, transition metals), semimetal, non-metal (halogens element, noble gas), chalcogen elements, lanthanide, and actinide.

In another embodiment of the invention, the nanoparticle or compound is composed of or comprises a chemical element selected from the group consisting of: hydrogen, lithium, sodium, potassium, rubidium, caesium, francium, beryllium, magnesium, calcium, strontium, barium, radium, scandium, yttrium, lanthanide, actinide, titanium, zirconium, hafnium, rutherfordium, vanadium, niobium, tantalum, dubnium, chromium, molybdenum, tungsten, seaborgium, manganese, technetium, rhenium, bohrium, iron, ruthenium, osmium, hessium, cobalt, rhodium, iridium, meitherium, nickel, palladium, platinum, darmstadtium, copper, silver, gold, roentgenium, zinc, cadmium, mercury, copernicum, boron, aluminium, gallium, indium, thallium, ununtrium, carbon, silicon, germanium, tin, lead, fleovium, nitrogen, phosphorus, arsenic, antimony, bismuth, ununpentium, oxygen, sulphur, selenium, tellurium, polonium, livermorium, fluorine, chlorine, bromine, iodine, astatine, ununseptium, helium, neon, argon, krypton, xenon, radon, ununoctium, lanthanum, cerium, praseodymium, neodymium, promethium, samarium, europium, gadolinium, terbium, dysprosium, holmium, erbium, thulium, ytterbium, lutetium, actinium, thorium, proctactinium, uranium, neptunium, plutonium, americium, curium, berkelium, californium, einsteinium, fermium, mendelevium, nobelium, and lawrencium.

In some cases, the nanoparticle or compound can also be composed of or comprise an alloy, a mixture, or an oxide of this(these) chemical element(s).

In some cases, the nanoparticle or compound can be composed of more than 10⁻⁵⁰, 10⁻²⁰, 10⁻¹⁰, 10⁻⁵, 10⁻², 1, 5, 10, 50, 75, 80, 90, 95 or 99% of one or several of this(these) element(s), where this percentage can represent or be the mass or number of this(these) chemical elements comprised in the nanoparticle or compound divided by the total number or total mass of all chemical elements comprised in the nanoparticle or compound.

In some other cases, the nanoparticle or compound can be composed of or comprise less than 99, 95, 80, 75, 50, 20, 10, 5, 2, 1, 10⁻¹ or 10⁻⁵% of one or several of this(these) chemical element(s).

In still some other cases, this(these) chemical element(s) is(are) comprised inside the nanoparticles or compound, or at the surface of the nanoparticle or compound, or in the mineral or central part of the nanoparticle or compound, or in the coating of the nanoparticle or compound.

In one embodiment of this invention, the nanoparticle or compound is not composed of or does not comprise at least one chemical element belonging to the family selected from the group consisting of: metals (alkali metal, alkaline earth metal, transition metals), semimetal, non-metal (halogens element, noble gas), chalcogen elements, lanthanide, actinide.

In another embodiment of the invention, the nanoparticle or compound is devoid of or does not comprise at least one chemical element selected from the group consisting of: hydrogen, lithium, sodium, potassium, rubidium, caesium, francium, beryllium, magnesium, calcium, strontium, barium, radium, scandium, yttrium, lanthanide, actinide, titanium, zirconium, hafnium, rutherfordium, vanadium, niobium, tantalum, dubnium, chromium, molybdenum, tungsten, seaborgium, manganese, technetium, rhenium, bohrium, iron, ruthenium, osmium, hessium, cobalt, rhodium, iridium, meitherium, nickel, palladium, platinum, darmstadtium, copper, silver, gold, roentgenium, zinc, cadmium, mercury, copernicum, boron, aluminium, gallium, indium, thallium, ununtrium, carbon, silicon, germanium, tin, lead, fleovium, nitrogen, phosphorus, arsenic, antimony, bismuth, ununpentium, oxygen, sulphur, selenium, tellurium, polonium, livermorium, fluorine, chlorine, bromine, iodine, astatine, ununseptium, helium, neon, argon, krypton, xenon, radon, ununoctium, lanthanum, cerium, praseodymium, neodymium, promethium, samarium, europium, gadolinium, terbium, dysprosium, holmium, erbium, thulium, ytterbium, lutetium, actinium, thorium, proctactinium, uranium, neptunium, plutonium, americium, curium, berkelium, californium, einsteinium, fermium, mendelevium, nobelium, and lawrencium.

In another embodiment of the invention, the nanoparticle or compound is not composed of or does not comprise an alloy, a mixture, or an oxide of this(these) chemical element(s).

In one embodiment of the invention, the nanoparticles is defined as a particle with a size in one dimension, which is larger than 10⁻¹, 1, 2, 5, 10, 20, 50, 70, 100, 200 or 500 nm. In some cases, a nanoparticle with a large size can have a larger coercivity and/or a larger remanent magnetization and/or can more strongly or more efficiently absorb the energy or power of the radiation than a nanoparticles with a small size. In some cases, the amount of energy or power absorbed by a nanoparticle is increased by a factor of more than 1.001, 1.01, 1.1, 1.2, 1.5, 2, 5, 10, 10³, 10⁵ or 10⁷ by increasing the size of the nanoparticles by a factor of more than 1.001, 1.01, 1.1, 1.2, 1.5, 2, 5, 10, 10³, 10⁵ or 10⁷.

In another embodiment of the invention, the nanoparticle is defined as a particle with a size in one dimension, which is lower than 10⁴, 10³, 10², 10, 1 or 10⁻¹ nm. A nanoparticle with a small size can more easily be administered, for example intravenously, or can enable the avoidance of some toxicity or side effects, such as embolism.

In still another embodiment of the invention, the nanoparticle size lies between 10⁻² and 10²⁰ nm, 10⁻² and 10⁴ nm, between 10⁻¹ and 10³ nm, or between 1 and 10² nm. This can be the case when the nanoparticles or nanoparticles assembly possesses a well-defined, preferentially narrow, distribution in sizes.

In still another embodiment of the invention, the nanoparticle size distribution is lower than 1000, 100, 75, 50, 25, 10, 5, 2 or 1 nm. A narrow nanoparticles size distribution may be desired to prevent aggregation, or to favor an organization in chains of the nanoparticles.

In some cases, the nanoparticle size distribution can correspond to or be: i) the different sizes that the nanoparticles can have, ii) the difference between the maximum size and minimum size that the nanoparticles can have, iii) the full width half maximum of the nanoparticle distribution in sizes.

In still another embodiment of the invention, the nanoparticle size distribution is larger than 10⁻⁵⁰, 10⁻²⁰, 10⁻¹⁰, 10⁻⁵, 10⁻¹, 1, 2, 5, 10, 25, 50, 75, 100 or 1000 nm. A large nanoparticles size distribution may in some cases enable nanoparticles to be eliminated more rapidly.

In another embodiment of the invention, the nanoparticles have a surface charge, which is larger than −200, −100, −50, −10, −5, 0.1, 1, 2, 5, 10, 50 or 100 mV, preferentially at a pH lower than 0.1, 1, 2, 3, 4, 5, 6, 7, 8, 9, 10, 11, 12, 13 or 14. Preferentially, a nanoparticle can have a large surface charge at low pH when it is surrounded by a coating that enables to reach such charge without being destroyed.

In another embodiment of the invention, the nanoparticles have a surface charge, which is lower than 10⁵, 10³, 100, 50, 10, 5, 2, 1, 0.1, 0, −5, −10, −50, −100 or −200 mV, preferentially at a pH larger than 0.1, 1, 2, 3, 4, 5, 6, 7, 8, 9, 10, 11, 12, 13 or 14. A nanoparticle can have a low surface charge at high pH when it is surrounded by a coating that enables to reach such charge without being destroyed.

In still another embodiment of the invention, the nanoparticles have a surface charge comprised between +200 mV and −200 mV, +100 mV and −100 mV, +50 mV and −50 mV, +40 mV et-40 mV, +20 mV and −20 mV, +10 mV and −10 mV, or between +5 mV and −5 mV, preferentially at a pH lower than 14, 13, 12, 11, 10, 9, 8, 7, 6, 5, 4, 3, 2, 1 or 0.1.

In still another embodiment of the invention, the nanoparticles have a surface charge comprised between +200 mV and −200 mV, +100 mV and −100 mV, +50 mV and −50 mV, +40 mV and −40 mV, +20 mV and −20 mV, +10 mV and −10 mV, or between +5 mV and −5 mV, preferentially at a pH larger than 0.1, 1, 2, 3, 4, 5, 6, 7, 8, 9, 10, 11, 12, 13 or 14.

In another embodiment of the invention, the nanoparticles have a weight or a mass, preferentially expressed in unit such as gram (g), kilogram (kg), or milligram (mg). A gram of nanoparticles can be a gram of metal such as iron comprised in the nanoparticles. The mass or weight of the nanoparticles can correspond to the mass or weight of one nanoparticle or to the mass or weight of an assembly of nanoparticles.

In an embodiment, the mass of the nanoparticles, preferentially of 1 nanoparticle or of an assembly of nanoparticles, is larger than 10⁻²⁰, 10⁻¹⁰, 10⁻⁵, 10⁻², 1, 10, 10³, 10⁹ or 10²⁰ gram. In some cases, a large nanoparticle mass may be desired to increase the quantity of compounds released by the nanoparticles after degradation, for example if a large mass enables to bind more compounds to the nanoparticles and then yield a large quantity of compounds released from the nanoparticles.

In an embodiment, the mass of the nanoparticles is lower than 10²⁰, 10¹⁰, 10⁵, 10², 1, 10⁻¹, 10⁻³, 10⁻⁹ or 10⁻²⁰ gram. In some cases, a low nanoparticles mass may be desired to prevent or minimize nanoparticles toxicity or to increase the quantity of compounds released by the nanoparticles after degradation, for example is a low mass enables more easily to break the bounds between the nanoparticles and the compounds.

In one embodiment of the invention, the nanoparticles, the suspension, composition, or assembly of nanoparticles is stable, preferentially during a lapse of time, preferentially being its stability duration, which is larger than 10⁻¹⁰, 5, 10, 10⁵⁰ or 10¹⁰⁰ minute(s). In some cases, the nanoparticles, the suspension, composition, or assembly of nanoparticles can be stable at a concentration of nanoparticles larger than 1, 5, 10, 50, 100, 200, 500 or 1000 mg of nanoparticles per mL of solvent, matrix, or body part surrounding or comprising the nanoparticles. In some cases, the nanoparticles, the suspension, composition, or assembly of nanoparticles can be stable when: i) the nanoparticles are not degraded or do not lose partly or fully their coating or can be administered to the body part, or ii) the optical density of the nanoparticles, the suspension, composition, or assembly of nanoparticles, preferentially measured at 480 nm or at another wavelength, preferentially fixed, does not decrease by more than 1, 5, 10, 50, 75 or 90% or by more than 10⁻¹⁰, 10⁻³, 10⁻¹, 0.5 or 0.7, within 1, 5, 10, 10³, 10⁷ or 10²⁰ seconds following homogenization or mixing or optical density measurement or absorption measurement of this suspension or composition. This percentage can be equal to (OD_{B}−OD_{A})/OD_{B} or OD_{A}/OD_{B}, where OD_{B} is the optical density of the nanoparticles, the suspension, composition, or assembly of nanoparticles measured before the homogenization or mixing or optical density measurement or absorption measurement of the nanoparticles, the suspension, composition, or assembly of nanoparticles and OD_{A} is the optical density of the nanoparticles, the suspension, composition, or assembly of nanoparticles measured after the homogenization or mixing or optical density measurement or absorption measurement of the nanoparticles, the suspension, composition, or assembly of nanoparticles.

In some cases, the nanoparticles can be suspended in a liquid or dispersed in a matrix or body part to yield a homogenous nanoparticle dispersion or a highly stable nanoparticle composition or suspension.

In one embodiment of the invention, the nanoparticles are arranged in chains comprising more than 1, 2, 3, 4, 5, 6, 7, 8, 9, 10, 15, 20, 25, 30, 35 or 40 nanoparticles.

In another embodiment of the invention, the nanoparticles are arranged in chains, which have: i) a length smaller than 2.10¹⁰, 2.10⁵, 2.10³ or 2.10² nm, or ii) a number of nanoparticles in each chain smaller than 10³, 10², 5 or 2. In some cases, short chains of nanoparticles may be desired or obtained, for example to favor nanoparticle internalization in cells or after partial or total destruction of long chains.

In another embodiment of the invention, the nanoparticles are arranged in chains, which have: i) a length longer than 10⁻¹, 1, 5, 10, 2.10², 2.10³ or 2.10⁵ nm, or ii) a number of nanoparticles in each chain larger than 2, 5, 10, 10² or 10³. In some cases, long chains of nanoparticles may be desired or obtained to increase the quantity of heat or compounds dissociated from the nanoparticles, preferentially under the application of the radiation or to prevent nanoparticles aggregation or enable uniform nanoparticles distribution.

In still another embodiment of the invention, the nanoparticles are arranged in chains, which have: i) a length between 10⁻¹ and 10¹⁰ nm, or between 1 and 10⁵ nm, or ii) a number of nanoparticles in each chain between 2 and 10⁵, 2 and 10³, 2 and 10², or between 2 and 50.

In still another embodiment of the invention, the nanoparticles are arranged in chains when they are bound or linked to each other or when the crystallographic directions of two adjacent nanoparticles in the chain are aligned, wherein such alignment is preferentially characterized by an angle between two crystallographic directions belonging to two adjacent nanoparticles in the chains of less than 90, 80, 70, 60, 50, 20, 10, 3, or 2° C. (degree).

Preferentially when the nanoparticles are biologically synthesized, the nanoparticles can be arranged in chains: i) inside the organism that synthesizes them, also designated as synthetizing living organism, or ii) outside this organism. Preferentially, nanoparticles are arranged in chains after or before their extraction or isolation from this organism.

In one embodiment of the invention, the nanoparticles are not arranged in chains.

In another embodiment of the invention, the nanoparticles are synthesized chemically or are not synthesized by a living organism when less than 1, 2, 5, 10 or 100 step(s) of their production, such as crystallization of iron oxide, stabilization of the iron oxide mineral, organization of the nanoparticles, involves or is due to a living organism. In some cases, a chemical synthesis can be defined as a synthesis involving a majority of steps, or more than 1, 2, 5 or 10 steps, or more than 1, 2, 5, 25, 50, 75 or 90% of steps, which involve chemical reactions occurring without the involvement of living organisms, or parts of living organisms such as DNA, RNA, proteins, enzymes, lipids.

In another embodiment of the invention, a chemical synthesis can be used to produce a chemical substance or compound that mimics, copies, or reproduces the compartment, organelle, or other biological material, wherein this chemical synthesis or chemical substance can be used or can result in the production of the nanoparticles. In some cases, the compartment, organelle, or other biological material, can be a lysosome, an endosome, a vesicle, preferentially biological material that has the capacity or the function either to dissolve or transform crystallized iron into free iron or to transform free iron into crystalized iron. In some cases, this transformation is partial and preferentially results in the destruction or formation of partly crystallized assembly of iron atoms or ions, or preferentially results in a mixture of crystallized iron and non-crystallized iron. In some cases, crystallized iron can be defined as an assembly of iron atoms or ions that leads to the presence of crystallographic planes, preferentially observable using a technique such as transmission or scanning electron microscopy as a characterization method, and free iron can preferentially be defined as one of several iron atoms or ions that do not lead to the presence of crystallographic planes, preferentially highlighted by the absence of diffraction patterns, using for example transmission or scanning electron microscopy as a characterization method.

The invention also relates to the particle according to the invention and pharmaceutical composition according to the invention, wherein the magnetosome is composed of:
an iron oxide, preferentially either magnetite, preferentially of chemical formula Fe₃O₄, or maghemite, preferentially of chemical formula Fe₂O₃, or an intermediate composition between maghemite and magnetite, or
an iron sulfide, preferentially greigite, preferentially of chemical formula Fe^{II}Fe^{III}₂S₄, wherein the iron oxide and/or iron sulfide is or are preferentially crystallized, partly or fully.

In one embodiment of the invention, the magnetosomes are nanoparticles synthesized by, originating from, extracted from, or isolated from magnetotactic bacteria.

In some cases, Fe^{II}Fe^{III}₂S₄ can be Fe₂S₄ or a derivative of Fe₂S₄ or a combination of Fe and S atoms.

In one embodiment of the invention, magnetotactic bacteria are selected from the group consisting of: Magnetospirillum magneticum strain AMB-1, magnetotactic coccus strain MC-1, three facultative anaerobic vibrios strains MV-1, MV-2 and MV-4, the Magnetospirillum magnetotacticum strain MS-1, the Magnetospirillum gryphiswaldense strain MSR-1, a facultative anaerobic magnetotactic spirillum, Magnetospirillum magneticum strain MGT-1, and an obligate anaerobe, and Desulfovibrio magneticus RS-1.

In one embodiment of the invention, a magnetotactic bacterium is defined as a bacterium able to synthesize magnetosomes, wherein these magnetosomes are preferentially characterized by at least one of the following properties: i) they are produced intracellularly, ii) they are magnetic, iii) they comprise a mineral, iv) their core is preferentially composed of a metallic oxide such as iron oxide, v) their core is surrounded by biological material such as lipids, proteins, endotoxins, which can preferentially be removed, vi) they are arranged in chains, vii) they produce heat under the application of an alternating magnetic field.

In one embodiment of the invention, the magnetosomes possess one or several property(ies) in common with the nanoparticles such as at least one magnetic, size, composition, chain arrangement, charge, core, mineral, coating, or crystallinity property.

In one embodiment of the invention, magnetosomes comprise the mineral part synthesized by magnetotactic bacteria, i.e. preferentially the crystallized iron oxide produced by these bacteria. In this case, magnetosomes or magnetosome mineral parts preferentially do not comprise proteins, lipids, endotoxins, or biological materials comprising carbon or do not comprise more or comprise less than 0.1, 1, 10, 30, 50 or 75% or percent in mass of carbon, which is/are produced by these bacteria.

The invention also relates to nanoparticles for use, wherein nanoparticles are or are assimilated to chemical analogues of magnetosomes.

In some cases, chemical analogues of magnetosomes can be synthesizes chemically and/or are not synthesized by magnetotactic bacteria.

In some cases, chemical analogues of magnetosomes possess at least 1, 2, 3, 4, 5, 6, 7, 8, 9, or 10 common property(ies) with the magnetosomes, where these common properties are preferentially a ferrimagnetic behavior, preferentially a coercivity larger that 10⁻⁵⁰, 10⁻¹⁰, 10⁻², 1, 5, 10 or 100 Oe at a temperature preferentially larger than 0, 5, 10, 50, 100, 200, 300, 500 or 1000 K, a large size, preferentially a size larger than 1, 5, 10, 20, 50 or 70 nm, and/or a chain arrangement, preferentially an arrangement of more than 1, 2, 5 or 10 nanoparticles in chain.

In one embodiment of the invention, the nanoparticles or magnetosomes are purified to remove more than 10, 50 or 90 percent or percent in mass of endotoxins and/or other biological material such as proteins or lipids originating from the synthetizing living organism or magnetotactic bacteria. In some other cases, the nanoparticles or magnetosomes are purified to remove less than 100, 99.9, 99, 95 or 90 percent or percent in mass of endotoxins and/or other biological material. This purification step preferentially yields purified nanoparticles or magnetosomes. In some cases, this percentage can be equal to (Q_{BP}−Q_{AP})/Q_{BP} or Q_{AP}/Q_{BP}, where Q_{BP} and Q_{AP} are the quantities of endotoxins, biological material, proteins, or lipids before and after the purification step, respectively.

In some cases, the purification step can consist in using a method or detergent(s) such as NaOH and/or KOH, which is/are preferentially mixed with the synthetizing living organism or magnetotactic bacteria or bacterial debris, preferentially to remove organic material or separate the organic material from the inorganic material comprised in the nanoparticles or magnetosomes and preferentially then be able to harvest the nanoparticle or magnetosome mineral, preferentially comprised in the nanoparticles or magnetosomes.

In some cases, the purified nanoparticles or magnetosomes are nanoparticle or magnetosome minerals.

In an embodiment of the invention, the nanoparticles according to the invention are drugs, medical devices, cosmetic products, biological products, products used for research purposes, or products used to determine the properties of biological samples.

In some cases, the nanoparticle(s) can comprise the compound(s).

In some other cases, the nanoparticles don't comprise the compound(s) Preferably, the nanoparticle is a magnetosome.

Preferably, in some cases, the magnetosome is composed of iron oxide, preferentially magnetite or maghemite or an intermediate composition between maghemite and magnetite.

Preferably, in some other cases, the magnetosome is composed of greigite.

Preferably, greigite and/or maghemite and/or maghemite, preferentially comprised in the core or central part of the magnetosome, is/are crystalized, partly or fully.

Preferably, the method for increasing the release of at least one compound comprises the initial nanoparticle chosen among:

i) a magnetosome composed of magnetite, maghemite, or an intermediate composition between magnetite and maghemite.
ii) a magnetosome composed of iron sulfide.

The invention also relates to the method according to the invention, wherein the initial nanoparticle is chosen among:
a magnetosome composed of iron oxide, preferentially either magnetite, preferentially of chemical formula Fe₃O₄, or maghemite, preferentially of chemical formula Fe₂O₃, or an intermediate composition between maghemite and magnetite, or
a magnetosome composed of iron sulfide, preferentially greigite, preferentially of chemical formula Fe^{II}Fe^{III}₂S₄,
   wherein the iron oxide and/or iron sulfide is or are preferentially crystallized, partly or fully.

In still another embodiment of the invention, the property(ies) or features, preferentially of the particle or method, preferentially described in each individual embodiment or section or sentence of this patent application can be combined to result in a combination of property(ies) or features, preferentially of the particle or method.

In an embodiment of the invention, the bond is selected from the group consisting of: i) the initial bond, i.e. the bond that preferentially binds or is between the initial compound and the initial nanoparticle and is preferentially not exposed to the alteration and the physico-chemical disturbance, ii) the altered bond, i.e. the bond that preferentially binds or is between the altered compound and the altered nanoparticle, and can preferentially be separated, dissociated, or released from the altered nanoparticle and/or compound by alteration, and is preferentially exposed to alteration, iii) the altered and disturbed bond, i.e. the bond that preferentially binds or is between the altered and disturbed compound and the altered and disturbed nanoparticle, and can preferentially be separated, dissociated, or released from the altered and disturbed nanoparticle and/or compound by alteration and physico-chemical disturbance and that is preferentially exposed to alteration and physico-chemical disturbance.

In one embodiment, the bond is the chemical bond. In some cases, a bond between the nanoparticle and the compound exists when the compound is bound to or not released from the nanoparticle. In some cases, a bond between the nanoparticle and the compound does not exist when the compound is released from or not bound to the nanoparticle.

In one embodiment of the invention, a bond between the compound and the nanoparticle is: i) a physical link, ii) a link comprising an assembly of atoms, molecules, or linking material, or iii) an interaction. In some cases, the physical link, link and/or interaction is/are measured or occur(s) or exist(s) between the nanoparticle and the compound. In some cases, the bond can prevent the diffusion, movement, of the compound, preferentially of the compound alone without the nanoparticle.

In some cases, the bond can belong to or be comprised in the compound and/or the nanoparticle.

In some other cases, the bond can be or be located in-between the compound and the nanoparticle.

In some cases, the nanoparticle, bond, and compound, can be, be associated with or correspond to the center, surface, internal location of the nanoparticle, bond, and compound, respectively.

In some cases, the body part can be the body part of the individual or the body part of the living organism, preferentially comprising the particle.

In some cases, the individual can be the living organism.

In one embodiment of the invention, the body part of the individual is or can be designated as the body part.

In an embodiment of the invention, the body part comprises more than or is an assembly of more than 1, 2, 5, 10, 10³, 10⁵, 10¹⁰, 10⁵⁰ or 10¹⁰⁰ cell(s), apparatus, tissue(s), organ(s), biomolecule(s), molecule(s), atom(s), entities(s), or biological material(s), preferentially as measured per cm³ of body part.

In another embodiment of the invention, the body part comprises less than or is an assembly of less than 10¹⁰⁰, 10⁵⁰, 10¹⁰, 10⁵, 10³, 10, 5, 2 or 1 cell(s), apparatus, tissue(s), organ(s), biomolecule(s), molecule(s), atom(s), entities(s), or biological material(s), preferentially as measured per cm³ of body part.

In another embodiment of the invention, the body part comprises between or is an assembly of between 1 and 10¹⁰⁰, 1 and 10¹⁰, or 1 and 10³ cell(s), apparatus, tissue(s), organ(s), biomolecule(s), molecule(s), atom(s), entities(s), or biological material(s), preferentially as measured per cm³ of body part.

In some cases, the apparatus, the tissue(s), organ(s), biomolecule(s), molecule(s), atom(s), entities(s), or biological material(s) can be the same or belong to an assembly comprising the same tissue(s), organ(s), biomolecule(s), molecule(s), atom(s), entities(s), or biological material(s).

In some other cases, the apparatus, the tissue(s), organ(s), biomolecule(s), molecule(s), atom(s), entities(s), or biological material(s) can be different or belong to an assembly comprising different tissue(s), organ(s), biomolecule(s), molecule(s), atom(s), entities(s), or biological material(s).

In still some other cases, the apparatus, the tissue(s), organ(s), biomolecule(s), molecule(s), atom(s), entities(s), or biological material(s) can belong to, originate from, be produced by a living organism.

In still some other cases, the apparatus, the tissue(s), organ(s), biomolecule(s), molecule(s), atom(s), entities(s), or biological material(s) don't: i), belong to, ii), originate from, or iii) are produced by a living organism.

In still some other cases, the body part is a whole or part of a living organism, preferentially after or before its birth, preferentially after or before its death.

In one embodiment of the invention, the living organism or body part is or comprises at least 1, 10, 10³, 10⁵, 10¹⁰ or 10¹⁰⁰ eukaryotic or prokaryotic cell(s), DNA, RNA, protein, lipid, biological material, cell organelle, cell nucleus, cell nucleolus, ribosome, endoplasmic reticulum, Golgi apparatus, chloroplast, or mitochondria.

In another embodiment of the invention, the living organism or body part is an individual, a mammal, a bird, a fish, a human, a plant, a fungi, or an archaea, preferentially a male or a female.

In some cases, the body part can be all or part of the head, neck, shoulder, arm, leg, knee, foot, hand, ankle, elbow, trunk, inferior members, or superior members. In some other cases, the body part can be or belong to an organ, the musculoskeletal, muscular, digestive, respiratory, urinary, female reproductive, male reproductive, circulatory, cardiovascular, endocrine, circulatory, lymphatic, nervous (peripheral or not), ventricular, enteric nervous, sensory, or integumentary system, reproductive organ (internal or external), sensory organ, endocrine glands. The organ or body part can be human skeleton, joints, ligaments, tendons, mouth, teeth, tongue, salivary glands, parotid glands, submandibular glands, sublingual glands, pharynx, esophagus, stomach, small intestine, duodenum, jejunum, ileum, large intestine, liver, gallbladder, mesentery, pancreas, nasal cavity, pharynx, larynx, trachea, bronchi, lungs, diaphragm, kidneys, ureters, bladder, urethra, ovaries, fallopian tubes, uterus, vagina, vulva, clitoris, placenta, testes, epididymis, vas deferens, seminal vesicles, prostate, bulbourethral glands, penis, scrotum, pituitary gland, pineal gland, thyroid gland, parathyroid glands, adrenal glands, pancreas, heart, arteries, veins, capillaries, lymphatic vessel, lymph node, bone marrow, thymus, spleen, gut-associated lymphoid tissue, tonsils, brain, cerebrum, cerebral hemispheres, diencephalon, brainstem, midbrain, pons, medulla, oblongata, cerebellum, spinal cord, choroid plexus, nerves, cranial nerves, spinal nerves, ganglia, eye, cornea, iris, ciliary body, lens, retina, ear, outer ear, earlobe, eardrum, middle ear, ossicles, inner ear, cochlea, vestibule of the ear, semicircular canals, olfactory epithelium, tongue, taste buds, mammary glands, or skin. The body part or organ can belong to the blood circulation or circulatory system.

In some cases, the body part can be or comprise: i) at least 1, 5, 10, 10³, 10⁵ or 10¹⁰ tumor(s), cancer(s), virus(es), bacterium/bacteria, or pathological cell(s), and/or ii) less than 10¹⁰, 10⁵, 10³, 10, 5 or 1 healthy cell(s). In this case, the body part can be designated as the infected body part.

In some other cases, the body part can be or comprise: i) less than 10¹⁰, 10⁵, 10³, 10, 5 or 1 tumor(s), cancer(s), virus(es), bacterium/bacteria, or pathological cell(s), and/or ii) at least 1, 5, 10, 10³, 10⁵ or 10¹⁰ healthy cell(s). In this case, the body part can be a non-infected body part or healthy body part and preferentially be the body part where the nanoparticle is located or administered.

In some cases, the body part can be the particle/compound/nanoparticle region or the region where the particle/compound/nanoparticle is located or administered.

In one embodiment of the invention, the body part is or comprises water, an excipient, a solution, a suspension, at least one chemical element, organic material, or gel, which can be synthetic, i.e. preferentially involve a human in at least one step of its production, or produced by a living organism.

Preferably, the body part of an individual, also designated as the body part, represents or is part of an individual or a whole individual, where the individual is preferentially a human, an animal, or an organism, preferentially a living or inactivated or dead organism, comprising at least one prokaryotic or eukaryotic cell.

In one embodiment of the invention, the body part is alive (or not), is any tissue, water, medium, substance, cell, organelle, organ protein, lipid, DNA, RNA, biological material, preferentially localized in a specific region of an individual, preferentially originating or extracted from such region.

In some cases, the body part can comprise pathological cells, such as tumor cells, bacteria, eukaryotic or prokaryotic cells, as well as viruses or other pathological material. Pathological cells can be cells that are: i) not arranged or working as they usual do in a healthy individual, ii) dividing more quickly than healthy cells, iii) healthy cells having undergone a transformation or modification, iv) dead, sometimes due to the presence of a virus or to other organisms, or v), in contact, in interaction, with foreign material not belonging to the individual, such as viruses, where viruses can possibly penetrate, colonize, or replicate in these cells. In some cases, pathological cells can be assimilated to viruses or to other organisms or entities that colonize cells or target cells or destroy cells or use cells or enter in interaction with cells, preferentially to enable their own reproduction, multiplication, survival, or death. In some cases, a pathological site can comprise healthy cells, preferentially with a lower number, activity or proliferation, than that of pathological cells.

In some cases, the body part can comprise healthy cells, where a healthy cell can be defined as a cell that belongs to a healthy individual or to the body part of a healthy individual.

In some cases, the number of pathological or healthy cells, preferentially comprised in the body part can be lower than 10¹⁰⁰, 10⁵⁰, 10²⁰, 10¹⁰, 10⁵, 10, 5, 2 or 1 cell(s) preferentially per cm³ of body part.

In some other cases, the number of pathological or healthy cells, preferentially comprised in the body part, can be larger than 1, 10, 10³, 10⁵, 10⁷, 10⁹, 10²⁰, 10⁵⁰ or 10¹⁰⁰ cell(s) preferentially per cm³ of body part.

In one embodiment of this invention, the body part is divided between a portion of the body part comprising the particles and a portion of the body part not comprising the particles.

In one embodiment of the invention, the body part is divided between a portion of the body part comprising healthy cells or a majority of healthy cells, which is preferentially the portion comprising the nanoparticles or the portion where the nanoparticles are administered or the nanoparticle region and a portion of the body part comprising pathological cells or virus or bacteria or a majority of pathological cells, virus or bacteria or cells/entities such as T, B, APC cells or MHC or cytokines that can kill pathological cells or virus or bacteria.

In some cases, the body part can comprise: i) more than 10⁻⁹, 10⁻⁷, 10⁻⁵, 10⁻³, 10⁻¹, 1, 10, 10³, 10⁵, 10⁷ or 10⁹ mg of particles, preferentially per mm³ or per cm³ of body part or per pathological or healthy cell, or ii) more than 10⁻⁹, 10⁻⁷, 10⁻⁵, 10⁻³, 10⁻¹, 1, 10, 10³, 10⁵, 10⁷ or 10⁹ pathological or healthy cells, preferentially per mm³ or per cm³ of body part.

In some other cases, the body part can comprise: i) less than 10⁻⁹, 10⁻⁷, 10⁻⁵, 10⁻³, 10⁻¹, 1, 10, 10³, 10⁵, 10⁷, 10⁹, 10⁵⁰ or 10¹⁰⁰ mg of particle(s) or particle(s), preferentially per mm³ or per cm³ of body part or per pathological or healthy cell, or ii) less than 10⁻⁹, 10⁻⁷, 10⁻¹, 10⁻³, 10⁻¹, 1, 10, 10³, 10¹, 10⁷ or 10⁹ cell(s), preferentially pathological or healthy cell(s), preferentially per mm³ or per cm³ of body part.

In another embodiment of the invention, the particles remain in the body part during the method or method of treatment, preferentially during more than 1, 2, 5, 10, 50, 100 or 10³ second(s), hour(s), day(s), month(s) or year(s).

In another embodiment of the invention, the nanoparticles remain in the body part during the method or method of treatment, preferentially during less than 1, 2, 5, 10, 50, 100 or 10³ second(s), hour(s), day(s), month(s) or year(s).

In some cases, the particles can remain in the body part during the method or method of treatment without decreasing in size by more than 10⁻⁴, 10⁻¹, 1, 10, 20, 50, 100, 500, 10³ or 10⁴% between before and after nanoparticle administration in/to the body part, where this percentage can be the ratio between the size of the nanoparticles after administration of the nanoparticles in the body part and the size of the nanoparticles before administration of the nanoparticles in the body part.

In some other cases, the nanoparticles can remain in the body part during the method where they decrease in size by more than 10⁻⁴, 10⁻¹, 1, 10, 20, 50, 100, 500, 10³ or 10⁴% between before and after particle administration in/to the body part.

In one embodiment of the invention, the particles are administered to or in the body part when they are directly administered to the body part or when they are administered close to the body part, preferentially less than 1, 10⁻¹, 10⁻², 10⁻³, 10⁻⁴, 10⁻⁵, 10⁻⁶ or 10⁻⁹ m away from the body part. In this case, the particles may not need to be transported or diffuse, for example in blood circulation, from the region or site where they are administered to the body part.

In another embodiment of the invention, the nanoparticles are administered to or in the body part, when they are indirectly administered to the body part or when they are administered far from the body part, preferentially more than 1, 10⁻¹, 10⁻², 10⁻³, 10⁻⁴, 10⁻⁵, 10⁻⁶ or 10⁻⁹ m away from the body part.

In this case, the nanoparticles may be transported or diffuse from the region or site where they are administered to the body part.

In still another embodiment of the invention, administering particles to or in the body part is the same as or comprises at least one of the steps of: i), localizing or having localized particles in the body part, ii) having particles diffuse or be transported to the body part, iii) transport particles to the body part, or iv) imaging particles, preferentially to verify that particles reach or are in the body part or that they are transported or diffusing towards the body part or that they are distributed or localized in the body part.

In another embodiment of the invention, the particles are administered to or in the body part when they are injected in, or mixed with, or introduced in, or inserted in the body part.

In another embodiment of the invention, the nanoparticles are administered to or in the body part when they occupy more than 10⁻⁹, 10⁻⁷, 10⁻⁵, 10⁻³, 1, 10, 25, 50 or 75%, preferentially by mass or volume, of the body part, where this percentage can be the ratio between the volume of the region occupied by the particles in the body part or nanoparticle region and the volume of the body part. This occupation can correspond to that measured 10⁻⁵, 10⁻³, 10⁻¹, 1, 10, 10³ or 10⁵ minute(s) following particle administration.

In another embodiment of the invention, the particles are administered to or in the body part following at least one of the following administration routes: local, enteral, gastrointestinal, parenteral, topical, oral, inhalation, intramuscular, subcutaneous, intra-tumor, in an organ, in a vein, in arteries, in blood, in tissue, a route used for vaccination.

The invention also relates to the method according to the invention, wherein the alteration of step a) is repeated a number of time Nₐ, wherein each alteration lasts for a time tₐ, wherein the physico-chemical disturbance of step b) is repeated a number of time N_{b}, wherein each physico-chemical disturbance lasts for a time t_{b}, wherein two different alterations are separated by a length of time tₐₐ, wherein two different physico-chemical disturbances are separated by a length of time t_{bb},

wherein Nₐ, N_{b}, tₐ, t_{b}, tₐₐ, and t_{bb} have at least one property selected in the group consisting of:
i) Nₐ is smaller than N_{b},
ii) Nₐ is equal to one,
iii) N_{b} is larger than one,
iv) tₐ is longer than t_{b}, and
v) t_{bb} is longer than tₐₐ.

Preferentially, Nₐ and N_{b} are integers.

In some cases, Nₐ and/or N_{b} can be larger than 0, 1, 2, 5, 10, 10³ or 10⁵.

In some other cases, Nₐ and/or N_{b} can be smaller than 10⁵, 10³, 10², 5, 2, 1 or 0.

In still some other cases Nₐ can be smaller than N_{b} by at least 0 1, 2, 3, 5, 10 or 10³.

In still some other cases N_{b} can be smaller than Nₐ by at least 0 1, 2, 3, 5, 10 or 10³.

In still some other cases, tₐ, t_{b}, tₐₐ or t_{bb} can be longer than 0, 10⁻²⁰, 10⁻¹, 1, 5, 10 or 10³ minute(s).

In still some other cases, tₐ, t_{b}, tₐₐ or t_{bb} can be shorter than 0, 10²⁰, 10⁵, 10, 1, 1, 10⁻³ or 10⁻⁵ minute(s).

In still some other cases, tₐ can be longer than t_{b} or t_{bb} can be longer than tₐₐ by at least 0, 10⁻⁵, 10⁻¹⁰, 10⁻⁵, 10⁻¹, 1, 5, 10 or 10³ minute(s).

In still some other cases, t_{b} can be longer than tₐ or tₐₐ can be longer than t_{bb} by at least 0, 10⁻⁵, 10⁻¹, 10⁻⁵, 10⁻¹, 1, 5, 10 or 10³ minute(s).

In one embodiment of the invention, two different alterations, where each alteration preferentially lasts for tₐ, are separated by a length of time tₐₐ during which no alteration is applied on the particle.

In one embodiment of the invention, two different physico-chemical disturbances, where each physico-chemical disturbance preferentially lasts for t_{b}, are separated by a length of time t_{bb} during which no physico-chemical disturbance is applied on the particle.

The invention also relates to the method according to the invention, wherein the alteration, which is applied on the first transforming particle transforming from the initial particle to the altered particle, and the physico-chemical disturbance, which is applied on the second transforming particle transforming from the altered particle to the altered and disturbed particle, have at least one property selected from the group consisting of i) to x):

i) The alteration is or is due to a first variation of pH of the first transforming particle or of its environment, which is larger than 10⁻³ pH units,
ii) The physico-chemical disturbance is or is due to a second variation of pH of the second transforming particle or of its environment, which is larger than 10⁻³ pH units,
iii) The alteration is or is due to a first variation of temperature of the first transforming particle or of its environment, which is larger than 10⁻³° C.,
iv) The physico-chemical disturbance is or is due to a second variation of temperature of the second transforming particle or of its environment, which is larger than 10⁻³° C.,
v) The physico-chemical disturbance is associated with a second internalization of the second transforming particle, which is an extension a first internalization of the first transforming particle due to alteration,
vi) The alteration is associated with the first transforming particle being brought in the presence of altering chemical or biological material,
vii) The physico-chemical disturbance is associated with the second transforming particle being brought in the presence of altering chemical or biological material,
viii) The alteration is due to a first radiation or to the application of a first radiation on the first transforming particle,
ix) The physico-chemical disturbance is due to a second radiation or to the application of a second radiation on the second transforming particle,
and
x) The physico-chemical disturbance is a second radiation that has a strength, power, frequency, and/or intensity that is/are larger than the strength, power, frequency, and/or intensity of the first radiation being the alteration,
and wherein preferentially the altering chemical or biological material is selected in the group consisting of: a) at least one denaturing material, where a denaturing material can be selected from a first material that induces a loss in crystallinity, activity or a reduction in size of a second material or a first material that induces unfolding such as protein unfolding or a loss in quaternary, ternary, secondary, first structure of a second material such as an enzyme or protein or a first material that induces a loss in sheet, preferentially β sheet, or helix, preferentially a helix, structures of a second material, b) at least one cell, cell organelle, protein, peptide, enzyme, DNA, RNA, DNA strand or base, RNA strand or base, part of any of these substances, preferentially denaturing, c) at least one detergent, d) at least one acid such as HCl, e) at least one base such as NaOH, f) at least one chaotropic agent, g) a compound with at least one chemical function selected in the group consisting of: carboxylic acids, phosphoric acids, sulfonic acids, esters, amides, ketones, alcohols, phenols, thiols, amines, ether, sulfides, acid anhydrides, acyl halides, amidines, nitriles, hydroperoxides, imines, aldehydes, and peroxides, h) Acetic acid, i) Alcohol, j) DMSO (Dimethylsulfoxyde), k) Ethanol, l) Formaldehyde, m) Formamide, n) Guanidine, o) Glutaraldehyde, p) Guanidinium chloride, q) Guanidine Thiocyanate, r) HCl, s) Lithium perchlorate, t) NaOH, u) Nitric Acid, v) Picric acid, w) Propylene glycol, x) Sodium bicarbonate, y) Sodium dodecyl sulfate, z) Sodium salicylate, aa) Sulfosalicylic acid, bb) Trichloroacetic acid, cc) Urea, dd) Polar solvent, ee) Apolar solvent, ff) an acidic, basic, oxidized, reduced, neutral, positively charged, negatively charged derivative of these compounds, and gg) a combination of several of these compounds or derivatives,
and wherein preferentially the first and/or second radiation(s) is/are selected in the group consisting of:
   a) electromagnetic radiation, b) acoustic radiation forces, c) radiation forces, d) radiation pressures, e) irradiation, preferentially of the body part, f) a source of radiation, g) a magnetic or electric field, h) an alternating magnetic or electric field, i) a magnetic or electric field gradient, j) light or laser light, k) light produced by a lamp, l) light emitted at a single wavelength, m) light emitted at multiple wavelengths, n) a ionizing radiation, o) microwave, p) radiofrequencies, q) acoustic wave, r) alpha, beta, gamma, X-ray, neutron, proton, electron, ion, neutrino, muon, meson, photon particles or radiation, s) infrasound, sound, ultra-sound, or hypersound, t) particle with a non-zero weight, and u) oscillating waves with a zero-weight,
   and wherein optionally, the strength or power or intensity of the first and/or second radiation is comprised between 10⁻¹⁰ and 10¹⁰ Watt, J, T, preferentially expressed per cm, cm² or cm³ of body part,
   and wherein optionally, the frequency of the first and/or second radiation is comprised between 10⁻¹⁰ and 10²⁰ Hz.

In some cases, the alteration can be applied on: i) the initial particle, ii) initial nanoparticle, iii) initial compound, iv) the first transforming particle transforming from the initial particle to the altered particle, iv) the first transforming compound transforming from the initial compound to the altered particle, v) the first transforming nanoparticle transforming from the initial nanoparticle to the altered nanoparticle, vi) the altered particle, vii) the altered nanoparticle, and/or viii) the altered compound.

In some cases, the transforming particle can be or comprise: i) the initial particle, ii) the first transforming particle, iii) the altered particle, iv) the second transforming particle, or v) the altered and disturbed particle.

In some cases, the physico-chemical disturbance can be applied on: i) the altered particle, ii) the altered nanoparticle, iii) the altered compound, iv) the second transforming particle transforming from the altered particle to the altered and disturbed particle, iv) the second transforming compound transforming from the altered compound to the altered and disturbed compound, v) the second transforming nanoparticle transforming from the altered nanoparticle to the altered and disturbed nanoparticle, vi) the altered and disturbed particle, vii) the altered and disturbed nanoparticle, and/or viii) the altered and disturbed compound.

In some cases, the altering chemical or biological material is or is not selected in the group consisting of: a) at least one denaturing material, where a denaturing material can be defined as a first material that induces a loss in crystallinity, activity or a reduction in size of a second material or a first material that induces unfolding such as protein unfolding or a loss in quaternary, ternary, secondary, first structure of a second material such as an enzyme or protein or a first material that induces a loss in sheet, preferentially β sheet, or helix, preferentially a helix, structures of a second material b) at least one cell, cell organelle, protein, peptide, enzyme, DNA, RNA, DNA strand or base, RNA strand or base, part of any of these substances, preferentially denaturing, c) at least one detergent, d) at least one acid such as HCl, e) at least one base such as NaOH, f) at least one chaotropic agent, g) a compound with at least one chemical function selected in the group consisting of: carboxylic acids, phosphoric acids, sulfonic acids, esters, amides, ketones, alcohols, phenols, thiols, amines, ether, sulfides, acid anhydrides, acyl halides, amidines, nitriles, hydroperoxides, imines, aldehydes, and peroxides, h) Acetic acid, i) Alcohol, j) DMSO (Dimethylsulfoxyde), k) Ethanol, l) Formaldehyde, m) Formamide, n) Guanidine, o) Glutaraldehyde, p) Guanidinium chloride, q) Guanidine Thiocyanate, r) HCl, s) Lithium perchlorate, t) NaOH, u) Nitric Acid, v) Picric acid, w) Propylene glycol, x) Sodium bicarbonate, y) Sodium dodecyl sulfate, z) Sodium salicylate, aa) Sulfosalicylic acid, bb) Trichloroacetic acid, cc) Urea, dd) Polar solvent, ee) Apolar solvent, ff) an acidic, basic, oxidized, reduced, neutral, positively charged, negatively charged derivative of these compounds, and gg) a combination of several of these compounds or derivatives.

In some cases, the physico-chemical disturbance can be associated with a second internalization of the second transforming particle, which is preferentially an extension of the alteration associated with the first internalization of the first transforming particle. This can occur when the particle is internalized in a cell or part of a cell such as an endosome or lysosome, this internalization preferentially starts during alteration and preferentially continues during physico-chemical disturbance.

In some other cases, the transforming particle being internalized only occurs during alteration or physico-chemical disturbance.

In some other cases, the transforming particle may be expelled from a cell or part of a cell during alteration and/or physico-chemical disturbance.

In some cases, the physico-chemical disturbance can be associated with the second transforming particle being brought in the presence of altering chemical or biological material, or an enzyme, wherein such event is preferentially an extension of the alteration associated with the first transforming particle being brought in the presence of altering chemical material, altering biological material, or an enzyme. This can occur when the particle is brought in the presence of altering chemical or biological material, or an enzyme, this event preferentially starts during alteration and preferentially continues during physico-chemical disturbance.

In some cases, the alteration can be associated with the first transforming particle being brought in the presence of altering chemical or biological material,

In some cases, the physico-chemical disturbance can be associated with the second transforming particle being brought in the presence of altering chemical or biological material,

In some other cases, the transforming particle being brought in the presence of altering chemical or biological material only occurs during alteration or physico-chemical disturbance.

In some cases, the altering chemical or biological material can be or comprise from the group consisting of: i) at least one denaturing material, where a denaturing material can be defined as a first material that induces a loss in crystallinity, activity or a reduction in size of a second material or a first material that induces unfolding such as protein unfolding or a loss in quaternary, ternary, secondary, first structure of a second material such as an enzyme or protein or a first material that induces a loss in sheet, preferentially β sheet, or helix, preferentially a helix, structures of a second material ii) at least one cell, cell organelle, protein, peptide, enzyme, DNA, RNA, DNA strand or base, RNA strand or base, part of any of these substances, preferentially denaturing, iii) at least one detergent, iv) at least one acid such as HCl, v) at least one base such as NaOH, vi) at least one chaotropic agent, vii) a compound with at least one chemical function selected in the group consisting of: carboxylic acids, phosphoric acids, sulfonic acids, esters, amides, ketones, alcohols, phenols, thiols, amines, ether, sulfides, acid anhydrides, acyl halides, amidines, nitriles, hydroperoxides, imines, aldehydes, and peroxides, viii) an acidic, basic, oxidized, reduced, neutral, positively charged, negatively charged derivative of these compounds, and ix) a combination of several of these compounds or derivatives.

In some cases, examples of altering chemical or biological material can comprise at least one compounds selected from the group consisting of: i) Acetic acid, ii) Alcohol, iii) DMSO (Dimethylsulfoxyde), iv) Ethanol, v) Formaldehyde, vi) Formamide, vii) Guanidine, viii) Glutaraldehyde, ix) Guanidinium chloride, x) Guanidine Thiocyanate, xi) HCl, xii) Lithium perchlorate, xiii) NaOH, xiv) Nitric Acid, xv) Picric acid, xvi) Propylene glycol, xvii) Sodium bicarbonate, xviii) Sodium dodecyl sulfate, xix) Sodium salicylate, xx) Sulfosalicylic acid, xxi) Trichloroacetic acid, xxii) Urea, xxiii) Polar solvent, xxiv) Apolar solvent, xxv) an acidic, basic, oxidized, reduced, neutral, positively charged, negatively charged derivative of these compounds, and xxi) a combination of several of these compounds or derivatives.

In some cases, alteration or physico-chemical disturbance can be carried out under at least one of the following conditions: i) mechanical agitation of the particle, ii) exposing the particle to radiation, iii) increasing or decreasing the temperature of the particle, preferentially by at least 10⁻¹⁰, 10⁻¹, 1, 5, 10 or 100° C., iv) denaturing preferentially thermally, chemically, or biologically the particle, v) beads milling the particle, and vi) sonicating, preferentially bath or probe sonicating, the particle.

In some cases, the altering chemical or biological material is brought into presence or mixed with the particle at a concentration larger than 0, 10⁻⁵⁰, 10⁻¹⁰, 10⁻⁵ or 10⁻¹ M.

In some other cases, the altering chemical or biological material is brought into presence or mixed with the particle at a concentration lower than 10⁵⁰, 10²⁰, 10⁵, 10, 5, 2, 1, 10⁻¹ or 10⁻³ M.

In some other cases, the particle is brought into presence or mixed with altering or biological material that is more concentrated, preferentially by a factor of at least 0, 1, 1.1, 2, 5 10 during alteration than during physico-chemical disturbance.

In some other cases, the particle is brought into presence or mixed with altering or biological material that is more concentrated, preferentially by a factor of at least 0, 1, 1.1, 2, 5 10 during physico-chemical disturbance than during alteration.

In some cases, the altering or biological material used during alteration is the same as the altering or biological material used during physico-chemical disturbance.

In some cases, the altering or biological material used during alteration is different from the altering or biological material used during physico-chemical disturbance.

In some cases, altering or biological material is used during alteration while altering or biological material is not used during physico-chemical disturbance.

In some cases, altering or biological material is not used during alteration while altering or biological material is used during physico-chemical disturbance.

In some cases, physico-chemical disturbance uses or comprises: i) altering or biological material, preferentially the same as that used during alteration, the concentration of this material preferentially differing between alteration and physico-chemical disturbance, and ii) preferentially a radiation that is added to the altering or biological material, preferentially applied in a repeated or sequential manner.

In some cases, the altering biological/chemical material can be the environment of the particle.

In some cases, the alteration and/or physico-chemical disturbance can be carried out by varying, increasing or decreasing the pH of the particle or of the environment or medium or altering biological/chemical material comprising the particle by a quantity ΔpH, also preferentially designated as the first and/or second pH variation of the particle, the environment or medium or altering biological/chemical material comprising the particle.

In some cases, the alteration and/or physico-chemical disturbance is or is due to ΔpH.

In some cases, the pH of the particle, preferentially the first or second transforming particle, or of the environment or medium or altering biological/chemical material comprising the particle is equal to 0+ΔpH or to 14−ΔpH.

In some cases, ΔpH is larger than 0, 10⁻⁵⁰, 10⁻¹⁰, 10⁻¹, 1, 2, 3, 4, 5, 6, 10, 14 or 20 pH unit.

In some other cases, ΔpH is larger than 0, 10⁻⁵, 10⁻¹⁰, 10⁻¹, 1, 2, 3, 4, 5, 6, 10, 14 or 20 pH unit.

In some cases, the first variation of the pH of the first transforming particle or its environment or the second variation of the pH of the second transforming particle or its environment can be larger than 10⁻¹⁰, 10⁻³, 10⁻¹, 0, 1, 5 or 10 pH unit(s).

In some other cases, the first variation of the pH of the first transforming particle or its environment or the second variation of the pH of the second transforming particle or its environment can be smaller than 10¹⁰, 10³, 1, 0.5 or 10⁻¹ pH unit(s).

In some cases, the first variation of the pH of the first transforming particle or its environment is larger than the second variation of the pH of the second transforming particle or its environment by at least 10⁻¹⁰, 10⁻³, 10⁻¹, 0, 1, 5 or 10 pH unit(s).

In some cases, the first variation of the pH of the first transforming particle or its environment is smaller than the second variation of the pH of the second transforming particle or its environment by at least 10⁻¹⁰, 10⁻³, 10⁻¹, 0, 1, 5 or 10 pH unit(s).

In some cases, the alteration and/or physico-chemical disturbance can be carried out by varying, increasing or decreasing the temperature of the particle or of the environment or medium or altering biological/chemical material comprising the particle by a quantity ΔT, also preferentially designated as the first and/or second temperature variation of the particle, the environment or medium or altering biological/chemical material comprising the particle.

In some cases, ΔT can be measured relatively to the temperature measured before alteration and/or physical-disturbance.

In some cases, the alteration and/or physico-chemical disturbance is/are due to ΔT.

In some cases, ΔT can be larger than 0, 10⁻²⁰, 10⁻¹⁰, 10⁻⁵, 10⁻¹, 1, 2, 5, 10, 50, 10² or 10³° C.

In some other cases, ΔT can be smaller than 10⁵⁰, 10¹⁰, 10⁵, 10³, 10², 75, 50, 10, 5, 2, 1 or 10⁻⁵° C.

In some cases, the alteration and/or physico-chemical disturbance can be carried out by applying a radiation on the particle, preferentially a first radiation on the first transforming particle, preferentially a second radiation on the second transforming particle.

In some cases, the alteration is or is to a first radiation applied on the first transforming particle.

In some cases, the physico-chemical disturbance is or is due to a second radiation applied on the second transforming particle.

In one embodiment, the strength, power, frequency, and/or intensity of the first radiation associated with or producing the alteration is larger, preferentially by a factor of at least 0, 0.5, 1, 1.1, 1.5, 10 or 10³, than the strength, power, frequency, and/or intensity of the radiation associated with or producing the physico-chemical disturbance.

In some cases, the strength, power, frequency, and/or intensity of the first or second radiation preferentially associated with or producing the alteration or physico-chemical disturbance is zero or smaller than 10⁵⁰, 10¹⁰, 10, 5, 2, 1 or 0 T, W, Hz, or J, optionally expressed per cm³, cm² or cm of body part.

In some other cases, the strength, power, frequency, and/or intensity of the first or second radiation preferentially associated with or producing the alteration or physico-chemical disturbance is non-zero or larger than 10⁻⁵⁰, 10⁻¹⁰, 10⁻¹, 1, 5, 10, 10³ or 10⁵ W, Hz, or J, optionally expressed per cm³, cm² or cm of body part.

In some cases, the strength or power or intensity of the first and/or second radiation can be comprised between 10⁻⁵⁰, 10⁻¹⁰, 10⁻⁵, 10⁻¹ or 0 Watt, J, T, preferentially expressed per cm, cm² or cm³ of body part and 0, 5, 10, 10³, 10⁵ or 10¹⁰ Watt, J, T, preferentially expressed per cm, cm² or cm³ of body part, In some cases, the frequency of the first and/or second radiation can be comprised between 10⁻²⁰, 10⁻¹, 10⁻⁵, 10⁻¹, 0 Hz and 1, 5, 10, 10⁵, 10¹⁰ and 10²⁰ Hz.

In some cases the physico-chemical disturbance has at least one property in common with the alteration.

In some other cases the alteration has at least one property different from that of the physico-chemical disturbance.

The invention also relates to the method according to the invention, wherein step a) and/or step b) is performed: i) through a cellular internalization, preferentially in a lysosome, ii) at an acidic pH, iii) at a temperature that differs by at least 1° C. from the physiological temperature, and/or iv) by or in the presence of an enzyme or degrading/altering biological/chemical material.

The invention also relates to the method according to the invention, wherein the quantity of compound released from the nanoparticle is larger than: i) 0, 10⁻¹⁰, 0.1, 1, 5, 10, 50 or 75% of the initial quantity of compound after step a), where the initial quantity of compound is the quantity of compound bond to the nanoparticle before performing step a), and/or ii) 10⁻¹⁰, 10⁻⁵, 10⁻¹, 0, 1, 5, 10 or 50% of the initial quantity of compound after step b), where the initial quantity of compound is the quantity of compound bound to the nanoparticle before performing step a) and/or step b).

The invention also relates to the method according to the invention, wherein the nanoparticle has a faculty to release compound which is higher when the said nanoparticle is altered and/or size-reduced, preferentially following or with or during step a), than when it is not altered and/or not size-reduced, preferentially before or without or not during step a).

The invention also relates to the method according to the invention, wherein the physico-chemical disturbance is chosen among: i) a variation of the environment of the altered particle comprising the altered nanoparticle and/or the altered compound and/or altered bond, and/or ii) a radiation applied on said altered particle.

In one embodiment of the invention, the physico-chemical disturbance is a variation of the environment of the particle, preferentially of the second transforming particle.

In some cases, the variation of the environment can be the variation of the environment of the first and/or second transforming particle.

In an embodiment of the invention, the variation of the environment of the particle according to the invention is a pH variation of this environment.

In some cases, the variation of the environment of the particle according to the invention can be a pH increase of this environment.

In one embodiment, the variation of the environment of the particle is not a variation in pH and/or in temperature of this environment.

In some other cases, the variation of the environment of the particle according to the invention can be a pH decrease of this environment.

In some cases, the magnitude of the pH variation, pH decrease, or pH increase is smaller than 14, 13, 12, 11, 10, 9, 8, 7, 6, 5, 4, 3, 2, 1, or 0.1 pH units,

In some other cases, the magnitude of the pH variation, pH decrease, or pH increase is larger than 10⁻⁵, 10⁻¹, 1, 2, 3, 4, 5, 6, 7, 8, 9, 10, 11, 12, 13 or 14 pH units.

In another embodiment of the invention, the variation of the environment of the particle according to the invention is a variation in temperature of this environment.

In some cases, the variation of the environment of the particle according to the invention is a temperature increase of this environment.

In some other cases, the variation of the environment of the particle according to the invention is a temperature decrease of this environment.

In some cases, the magnitude of the temperature variation, temperature decrease, or temperature increase, preferentially of the environment of the particle, particle, compound and/or nanoparticle, can be lower than 10⁵⁰, 10¹⁰, 10³, 500, 400, 300, 200, 100, 50, 25, 10, 5, 1, or 0.1° C., preferentially per cm³ of body part or per mL or cm³ of suspension or matrix or region comprising the particle.

In some other cases, the magnitude of the temperature variation, temperature decrease, or temperature increase, preferentially of the environment of the particle, particle, compound and/or nanoparticle, can be larger than 10⁻⁵⁰, 10⁻¹⁰, 10⁻⁵, 10⁻¹, 1, 5, 10 or 10²° C., preferentially per cm³ of body part or per mL or cm³ of suspension or matrix or region comprising the particle.

In another embodiment of the invention, the variation in the environment of the particle according to the invention is a variation in redox potential of this environment,

In some cases, the variation of the environment of the particle according to the invention is an increase in redox potential of this environment.

In some other cases, the variation of the environment of the particle according to the invention is a decrease in redox potential of this environment.

In some cases, the magnitude of the redox potential variation, redox potential increase, or redox potential decrease is lower than 1000, 100, 10, 5, 2, 1, 0.1 or 0.01 V.

In some other cases, the magnitude of the redox potential variation, redox potential increase, or redox potential decrease is larger than 10⁻⁵⁰, 10⁻¹⁰, 10⁻⁵, 10⁻², 10⁻¹, 1, 5 or 10 V.

In an embodiment of the invention, the variation of the environment of the particle according to the invention is a variation in viscosity, preferentially in dynamic viscosity of this environment.

In some cases, the variation of the environment of the particle according to the invention is an increase in viscosity of this environment.

In some other cases, the variation of the environment of the particle according to the invention is a decrease in viscosity of this environment.

In some cases, the magnitude of viscosity variation, viscosity increase, or viscosity decrease can be lower than 10²⁰, 10¹⁰, 10⁵, 10³, 10², 10, 10⁻¹, 10⁻², 10⁻³, 10⁻⁶, 10⁻⁹ or 10⁻²⁰ Pa·s.

In some other cases, the magnitude of viscosity variation, viscosity increase, or viscosity decrease can be larger than 10⁻²⁰, 10⁻¹⁰, 10⁻⁵, 10⁻³, 10⁻², 10⁻¹, 1, 5, 10, 10³, 10⁶, 10⁹ or 10²⁰ Pa·s.

In an embodiment of the invention, the variation of the environment of the particle according to the invention is a variation in chemical composition of this environment, preferentially a variation in concentration of at least one substance in this environment.

In some cases, the variation of the environment of the particle according to the invention can be an increase of the concentration of at least one substance in this environment.

In some other cases, the variation of the environment of the particle according to the invention can be a decrease of the concentration of at least one substance in this environment.

In some cases, the magnitude of the variation, increase, or decrease of the concentration of at least one substance in this environment is lower than 10⁵⁰, 10²⁰, 10¹⁰, 10⁵, 100, 10, 1, 10⁻¹, 10⁻², 10⁻³, 10⁻⁶ or 10⁻⁹ M, mole per liter, micromole per liter, nano-mole per liter, mole per milliliter, micromole per milliliter, nano-mole per milliliter, mole per cubic meter, mole per cubic decimeter, mole per cubic centimeter, or mole per cubic millimeter, preferentially comprised in the body part, or in the suspension, matrix, or region comprising the particle.

In still some other cases, the magnitude of the variation, increase, or decrease of the concentration of at least one substance in this environment is larger than 10⁻⁵⁰, 10⁻²⁰, 10⁻¹⁰, 10⁻⁵, 10⁻¹, 1, 10, 10², 10³, 10⁶ or 10⁹ M, mole per liter, micromole per liter, nano-mole per liter, mole per milliliter, micromole per milliliter, nano-mole per milliliter, mole per cubic meter, mole per cubic decimeter, mole per cubic centimeter, or mole per cubic millimeter, preferentially comprised in the body part, or in the suspension, matrix, or region comprising the particle.

In one embodiment of the invention, the variation of the environment of the particle according to the invention is a modification of at least 1, 2, 3, 4, 5, 10, 25, 50, 100, 500, 10³, 10⁴, 10⁵, 10⁶, 10⁷, 10⁸, 10⁹ or 10¹⁰ substance (s) comprised in this environment, where this modification may be a chemical or structural modification and/or the appearance or disappearance of substance(s) from that environment. In one embodiment of the invention, the variation of the environment of the particle according to the invention is a modification of less than 10⁵⁰, 10¹⁰, 10⁵, 10, 5, 2 or 1 substance(s) comprised in this environment.

In one embodiment of the invention, the variation of the environment of the particle according to the invention is an increase in the concentration of radical or reactive species by a factor of at least 1.001, 1.1, 1.2, 2, 5, 10 or 10³, preferentially per cm³ of body part.

In one embodiment of the invention, the variation of the environment of the particle induces, produces, generates, is associated with, corresponds to a variation of pH, temperature, redox potential, viscosity, chemical composition, concentration of radical or reactive species of the particle, which can in some cases be larger for the particle than for its environment, which can in some other cases be lower for the particles than for its environment.

In some cases, the radiation can be the first and/or second radiation(s).

According to the invention, the radiation, preferentially the first and/or second radiation(s), can be: i) electromagnetic radiation, ii) acoustic radiation forces, iii) radiation forces, iv) radiation pressures, v) irradiation, preferentially of the body part, vi) a source of radiation, vii) a magnetic or electric field, viii) an alternating magnetic or electric field, ix) a magnetic or electric field gradient, x) light or laser light, xi) light produced by a lamp, xii) light emitted at a single wavelength, xiii) light emitted at multiple wavelengths, xiv) a ionizing radiation, xv) microwave, xvi) radiofrequencies, xvii) acoustic wave, xviii) alpha, beta, gamma, X-ray, neutron, proton, electron, ion, neutrino, muon, meson, photon particles or radiation, xix) infrasound, sound, ultra-sound, or hypersound, xx) particle preferentially with a non-zero weight, and xxi) oscillating waves preferentially with a zero-weight.

In one embodiment, the radiation, preferentially the first and/or second radiation(s), is not at least one of the following radiations: i) electromagnetic radiation, ii) acoustic radiation forces, iii) radiation forces, iv) radiation pressures, v) irradiation, preferentially of the body part, vi) a source of radiation, vii) a magnetic or electric field, viii) an alternating magnetic or electric field, ix) a magnetic or electric field gradient, x) light or laser light, xi) light produced by a lamp, xii) light emitted at a single wavelength, xiii) light emitted at multiple wavelengths, xiv) a ionizing radiation, xv) microwave, xvi) radiofrequencies, xvii) acoustic wave, xviii) alpha, beta, gamma, X-ray, neutron, proton, electron, ion, neutrino, muon, meson, photon particles or radiation, xix) infrasound, sound, ultra-sound, or hypersound, xx) particle preferentially with a non-zero weight, and xxi) oscillating waves preferentially with a zero-weight.

In some cases, the radiation according to the invention can have a strength larger than 1 μT, 10 μT, 100 μT, 1 mT, 10 mT, 100 mT, 1 T, 0 T, 5 T, 10 T or 100 T.

In some other cases, the radiation according to the invention can have a strength lower than 10²⁰, 10⁵, 10², 10, 1, 0, 10⁻¹, 10⁻³ or 10⁻¹ T.

In some other cases, the radiation according to the invention can have a power larger than 10⁻¹⁰, 10⁻⁵, 10⁻³, 0.01, 0.1, 0, 1, 10, 10², 10³, 10⁵ or 10⁷ Gy or Gy per cm³ of body part or Gy per gram of body part or Gy per cm³ of particle or Gy per gram of particle or Watt or Watt per cm³ of body part or Watt per gram of body part or Watt per cm³ of particle or Watt per gram of particle.

In some other cases, the radiation according to the invention can have a power lower than 10¹⁰⁰, 10⁵⁰, 10¹⁰, 10⁵, 10², 10, 1, 0, 10⁻³ or 10⁻⁵ Gy or Gy per cm³ of body part or Gy per gram of body part or Gy per cm³ of particle or Gy per gram of particle or Watt or Watt per cm³ of body part or Watt per gram of body part or Watt per cm³ of particle or Watt per gram of particle.

In some other cases, the radiation according to the invention can have an energy larger than 10⁻¹⁰, 10⁻⁵, 10⁻³, 0.01, 0.1, 0, 1, 10, 10², 10³, 10⁵ or 10⁷ J or J per cm³ of body part or J per gram of body part or J per cm³ of particle or J per gram of particle.

In some other cases, the radiation according to the invention can have a power lower than 10¹⁰⁰, 10⁵⁰, 10¹⁰, 10⁵, 10², 10, 1, 0, 10⁻³ or 10⁻⁵ J or J per cm³ of body part or J per gram of body part or J per cm³ of particle or J per gram of particle.

In one embodiment of the invention, the radiation is applied during a lapse of time larger than 10⁻⁵⁰, 10⁻¹⁰, 10⁻⁵, 10⁻¹, 0, 1, 5, 10, 10³, 10⁵ or 10¹⁰ second(s), minute(s), hour(s), day(s), month(s) or year(s).

In another embodiment of the invention, the radiation is applied during a lapse of time smaller than 10⁵⁰, 10¹⁰, 10⁵, 10², 5, 2, 1, 0, 10⁻¹, 10⁻⁵ or 10⁻¹⁰ second(s), minute(s), hour(s), day(s), month(s) or year(s).

The invention also relates to the method according to the invention, wherein:
step a) comprises the activation of the first part of the altered compound released from the altered nanoparticle by breaking of the initial bond, and/or
step b) comprises the activation of the second part of the altered compound bound to the altered nanoparticle via an altered bond.

The invention also relates to the method according to the invention, wherein:
step a) comprises the activation of the altered compound released from the altered nanoparticle by breaking of the initial bond
   and/or
step b) comprises the activation of the altered and disturbed compound released from the altered and disturbed nanoparticle by breaking of the altered and disturbed bond,
   wherein this activation is preferentially associated with or preferentially results in or leads to at least one event selected from the first series of events a) to c):
   a) the migration or diffusion or location of the released altered compound or of the released altered and disturbed compound towards or in an infected body part comprising at least one pathological cell, cancer cell, cell able to destroy pathological cell, virus, or bacterium,
   b) the destruction, attenuation, and/or prevention of division or replication of at least one pathological cell, cancer cell, virus, or bacterium, by the released altered compound or by the released altered and disturbed compound,
   and
   c) the interaction of the released altered compound or of the released altered and disturbed compound with a biological entity that triggers the production of substances such as antibodies that destroy or attenuate or prevent the division or replication of at least one pathological cell, cancer cell, virus, or bacterium, where such entity can be selected from: i) an immune cell, preferentially a B cell or an antigen presenting cell, and ii) a complex such as a major histocompatibility complex,
   wherein this activation is preferentially associated with or preferentially results in or leads to at least one event selected from the second series of events d) to f):
   d) an absence of migration or diffusion or location of the non-released altered compound, of the non-released altered and disturbed compound, of the altered nanoparticle, and/or of the altered and disturbed nanoparticle towards or in a body part region comprising at least one pathological cell, cancer cell, cell able to destroy pathological cell, virus, or bacterium, wherein d) is optionally achieved by applying a magnetic field or magnetic field gradient on the initial nanoparticle, altered nanoparticle, or altered and disturbed nanoparticle to maintain this nanoparticle in the body part where it is administered and/or to prevent its diffusion towards the infected body part,
   e) an absence of destruction, attenuation, and/or prevention of division or replication of at least one pathological cell, cancer cell, virus, or bacterium, the non-released altered compound, of the non-released altered and disturbed compound, of the altered nanoparticle, and/or of the altered and disturbed nanoparticle,
   and
   f) an absence of interaction of the non-released altered compound, of the non-released altered and disturbed compound, of the altered nanoparticle, and/or of the altered and disturbed nanoparticle, with a biological entity that triggers the production of substances such antibodies that destroy or attenuate or prevent the division or replication of at least one pathological cell, cancer cell, virus, or bacterium, where such entity can be selected from: i) an immune cell, preferentially a B cell or an antigen presenting cell, and ii) a complex such as a major histocompatibility complex.

In some cases, the diffusion of the released compound can occur between the nanoparticle region or the region where the nanoparticles are located or administered and the infected body part.

In some cases, the nanoparticles can be prevented from diffusing to the infected body part or to other regions than the region where they are administered by using a magnet or a magnetic field that preferentially attracts the nanoparticles and preferentially prevents nanoparticle diffusion or departure from the region where they are administered.

In some cases, the system consisting of the nanoparticle preferentially non-diffusing and/or the compound preferentially diffusing can behave or be like a patch or a system or medical device or drug enabling the slow or continuous or multiple release of the compound. In some cases, the activity of such system can be controlled by: i) a magnet or magnetic field that prevents the diffusion of nanoparticles, which are preferentially magnetic, and/or ii) the alteration and/or physico-chemical disturbance that can enable the compound to diffuse and preferentially trigger a medical activity. In some cases it can be interesting to prevent nanoparticle diffusion to avoid that nanoparticles induce toxicity by diffusing towards some parts of the organism such as lungs, liver, heart or brain. In some other cases, it can be interesting to prevent nanoparticle diffusion to exert a control on the interaction between the compound and the nanoparticle, i.e. if the nanoparticles are not moving it can be easier to apply the physico-chemical disturbance on the nanoparticle. In some other cases, it can be interesting to prevent nanoparticle diffusion so that the compound and the nanoparticle are separated by a sufficiently large distance, which is preferentially larger than the nanoparticle diameter, so that the compound can be fully active, so that the activity of the compound is not prevented by the binding of some active parts of the compound to the nanoparticle.

In some cases, the system or the particle is a vaccine or is comprised in a vaccine, where the vaccine preferentially enables multiple or repetitive or controlled activation of the immune system, preferentially through the activation/diffusion of the compound, preferentially through the use of the nanoparticles acting like a reservoir or pool of compounds. This system or particle can preferentially act against a virus, bacterium, or pathological cell, preferentially repetitively, preferentially until disappearance of the infection.

In some cases, the vaccine can be a nanoparticulate vaccine, preferentially a vaccine in which the particle is an adjuvant, preferentially a vaccine that can be activated or whose activity can be enhanced or produced through the heat or excitation produced under the application of a radiation on the nanoparticles, preferentially a vaccine that can trigger an effective response of the immune system preferentially against a virus, bacterium or pathological cell preferentially to destroy the virus, bacterium or pathological cell preferentially through the excitation, activation of the particle or release of the compound.

In some cases, the medical activity of the particle or the treatment efficacy of the particle against a disease is larger when the method or at least one step of the method is used or is followed than when the method or at least one step of the method is not used or is not followed.

In some cases, activity can mean medical, cosmetic, therapeutic, or diagnostic activity.

The invention also relates to the particle according to the invention for use in the treatment of a disease, preferentially an infectious disease, most preferentially cancer, virus or bacterial infections.

In one embodiment of the invention, the disease is or designates an infectious disease.

In one embodiment of the invention, the disease, preferentially the infectious disease, is due to, originates from, or is associated with the presence in the body part of: i), bacteria, preferentially pathological bacteria, ii), viruses, iii), tumor cells or, iv), foreign biological material not belonging to the living organism or body part.

In one embodiment of the invention, the disease is selected from the group consisting of: a malfunction of the living organism or body part, a disease associated with a proliferation of cells that is different from the cellular proliferation in a healthy individual, a disease associated with the presence of pathological cells in the body part, a disease associated with the presence of a pathological site in an individual or body part, a disease or disorder or malfunction of the body part, a disease associated with the presence of radio-resistant or acoustic-resistant or radiation-resistant cells, an infectious disease, an auto-immune disease, a neuropathology, a cancer, a tumor, a disease comprising or due to at least one cancer or tumor cell, one virus, one bacterium, a cutaneous condition, an endocrine disease, an eye disease or disorder, an intestinal disease, a communication disorder, a genetic disorder, a neurological disorder, a voice disorder, a vulvovaginal disorder, a liver disorder, a heart disorder, a heating disorder, a mood disorder, anemia, preferentially iron anemia, and a personality disorder.

In some cases, the disease or disorder can be the disease or disorder of or belonging to the individual or body part, or the disease or disorder from which the individual is suffering.

In one embodiment of the invention, the cancer or tumor is selected from the group consisting of: the cancer of an organ, cancer of blood, cancer of a system of a living organism, adrenal cancer, anal cancer, bile duct cancer, bladder cancer, bone cancer, brain cancer, breast cancer, cervical cancer, colon/rectum cancer, endometrial cancer, esophagus cancer, eye cancer, gallbladder cancer, heart cancer, kidney cancer, laryngeal and hypopharyngeal cancer, leukemia, liver cancer, lung cancer, nasal cavity and paranasal sinus cancer, nasopharyngeal cancer, neuroblastoma, non-Hodgkin lymphoma, oral cavity and oropharyngeal cancer, osteosarcoma cancer, ovarian cancer, pancreatic cancer, pancreatic penile cancer, prostate cancer, retinoblastoma, rhabdomyosarcoma, salivary gland cancer, sarcoma, skin cancer, small intestine cancer, stomach cancer, testicular cancer, thymus cancer, thyroid cancer, uterine cancer, uterine sarcoma cancer, vaginal cancer, vulvar cancer, waldenstrom macroglobulinemia wilms tumor, castleman disease ewing family of tumor, gastrointestinal carcinoid tumor, gastrointestinal stromal tumor, myelodysplastic syndrome pituitary tumor, and a cancerous disease such as gestational trophoblastic disease, Hodgkin disease, kaposi sarcoma, malignant mesothelioma, and multiple myeloma.

In one embodiment, the virus, which is preferentially responsible for the disease, is selected in the group consisting of virus or virus family: Abyssoviridae, Ackermannviridae, Adenoviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Bornaviridae, Botourmiaviridae, Bromoviridae, Caliciviridae, Carmotetraviridae, Caulimoviridae, Chrysoviridae, Chuviridae, Circoviridae, Clavaviridae, Closteroviridae, Coronaviridae, Coronavirus, COVID, COVID-19, Corticoviridae, Cruliviridae, Cystoviridae, Deltaflexiviridae, Dicistroviridae, Endornaviridae, Euronivirida, Filoviridae, Fimoviridae, Flaviviridae, Fuselloviridae, Gammaflexiviridae, Geminiviridae, Genomoviridae, Globuloviridae, Guttaviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Lavidaviridae, Leishbuviridae, Leviviridae, Lipothrixviridae, Lispiviridae, Luteoviridae, Malacoherpesviridae, Marnaviridae, Marseilleviridae, Matonaviridae, Medioniviridae, Megabirnaviridae, Mesoniviridae, Metaviridae, Microviridae, Mimiviridae, Mononiviridae, Mymonaviridae, Myoviridae, Mypoviridae, Nairoviridae, Nanoviridae, Narnaviridae, Nimaviridae, Nodaviridae, Nudiviridae, Nyamiviridae, Orthomyxoviridae, Ovaliviridae, Papillomaviridae, Paramyxoviridae, Partitiviridae, Parvoviridae, Peribunyaviridae, Permutotetraviridae, Phasmaviridae, Phenuiviridae, Phycodnaviridae, Picobirnaviridae, Picornaviridae, Plasmaviridae, Pleolipoviridae, Pneumoviridae, Podoviridae, Polycipiviridae, Polydnaviridae, Polyomaviridae, Portogloboviridae, Pospiviroidae, Potyviridae, Poxviridae, Pseudoviridae, Qinviridae, Quadriviridae, Reoviridae, Retroviridae, Rhabdoviridae, Roniviridae, Rudiviridae, Sarthroviridae, Secoviridae, Siphoviridae, Smacoviridae, Solemoviridae, Solinviviridae, Sphaerolipoviridae, Spiraviridae, Sunviridae, Tectiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, and Yueviridae.

In one embodiment, the bacterium, preferentially pathogenic bacterium, which is preferentially responsible for the disease, is selected from the group consisting of bacterium or bacterium family: Bacillus, Bacillus anthracis, Bacillus cereus, Bartonella, Bartonella henselae, Bartonella quintana, Bordetella, Bordetella pertussis, Borrelia, Borrelia burgdorferi, Borrelia garinii, Borrelia afzelii, Borrelia recurrentis, Brucella, Brucella abortus, Brucella canis, Brucella melitensis, Brucella suis, Campylobacter, Campylobacter jejuni, Chlamydia, Chlamydophila, Chlamydia pneumoniae, Chlamydia trachomatis, Chlamydophila psittaci, Clostridium, Clostridium botulinum, Clostridium difficile, Clostridium perfringens, Clostridium tetani, Corynebacterium, Corynebacterium diphtheriae, Enterococcus, Enterococcus faecalis, Enterococcus faecium, Escherichia, Escherichia coli, Francisella, Francisella tularensis, Haemophilus, Haemophilus influenzae, Helicobacter, Helicobacter pylori, Legionella, Legionella pneumophila, Leptospira, Leptospira interrogans, Leptospira santarosai, Leptospira weilii, Leptospira noguchii, Listeria, Listeria monocytogenes, Mycobacterium, Mycobacterium leprae, Mycobacterium tuberculosis, Mycobacterium ulcerans, Mycoplasma, Mycoplasma pneumoniae, Neisseria, Neisseria gonorrhea, Neisseria meningitides, Pseudomonas, Pseudomonas aeruginosa, Rickettsia, Rickettsia rickettsia, Salmonella, Salmonella typhi, Salmonella typhimurium, Shigella, Shigella sonnei, Staphylococcus, Staphylococcus aureus, Staphylococcus epidermidis, Staphylococcus saprophyticus, Streptococcus, Streptococcus agalactiae, Streptococcus pneumoniae, Streptococcus pyogenes, Treponema, Treponema pallidum, Ureaplasma, Ureaplasma urealyticum, Vibrio, Vibrio cholera Yersinia, Yersinia pestis Yersinia enterocolitica Yersinia pseudotuberculosis, Actinomyces israelii, Bacillus anthracis, Bacteroides fragilis, Bordetella pertussis, Borrelia, B. burgdorferi, B. garinii, B. afzelii, B. recurrentis, Brucella, B. abortus, B. canis, B. melitensis, B. suis, Campylobacter jejuni, Chlamydia, C. pneumoniae, C. trachomatis, Chlamydophila psittaci, Clostridium, C. botulinum, C. difficile, C. perfringens, C. tetani, Corynebacterium diphtheria, Ehrlichia, E. canis, E. chaffeensis, Enterococcus, E. faecalis, E. faecium, Escherichia, E. coli, Enterotoxigenic E. coli, Enteropathogenic E. coli, Enteroinvasive E. coli, Enterohemorrhagic E. coli, Francisella tularensis, Haemophilus influenzae, Helicobacter pylori, Klebsiella pneumoniae, Legionella pneumophila, Leptospira species, Listeria monocytogenes, Mycobacterium, M. leprae, M. tuberculosis, Mycoplasma pneumoniae, Neisseria, N. gonorrhoeae, N. meningitides, Pseudomonas aeruginosa, Nocardia asteroides, Rickettsia rickettsii, Salmonella, S typhi, Other Salmonella species, S. typhimurium, Shigella, S. sonnei, S. dysenteriae, Staphylococcus, aureus, epidermidis, saprophyticus, Streptococcus, agalactiae, pneumoniae, pyogenes, viridans, Treponema pallidum subspecies pallidum, Vibrio cholerae, and Yersinia pestis.

In one embodiment of the invention, the disorder or malfunction of the body part is associated with the malfunction of cells, which divide more rapidly or enter in an apoptotic or necrotic state for example, or with the malfunction of the immune system or immune cell(s).

In one embodiment of the invention, the method according to the invention is a medical treatment or medical diagnosis or medical method, which preferentially detects diagnoses, heals, or cures a disease such as one of those mentioned in the previous embodiments.

In one embodiment of the invention, the activation, preferentially the activation of the compound, nanoparticle and/or particle, is or is associated with or results in: i) a medical activity of the compound, nanoparticle, and/or particle, ii) the release of the compound from the nanoparticle, iii) the production of heat or temperature increase, preferentially of the environment of the particle, particle, compound and/or nanoparticle, iv) the temperature decrease, preferentially of the environment of the particle, particle, compound and/or nanoparticle, v) the production of radical or reactive species, preferentially by the particle, compound and/or nanoparticle.

In some cases, the medical activity can be or be associated with or result in: i) the healing or cure of the body part, ii) the decrease in number of cells, preferentially pathological cells, comprised in the body part, iii) the detection of the body part, or iv) the administration of the particle to/in the body part.

Preferably, the method for increasing the release of at least one compound comprising the alteration of step a) of the initial nanoparticle follows a kinetic or behavior of alteration having at least one property selected from the group comprising:
a reduction in particle size being of at least 1 nm when the pH of the altering medium and/or body part is decreased by at least 1 pH unit,
a disappearance or reduction in number of particles, preferentially of particles smaller than 25 nm, when the altering medium and/or body part has pH lower than 7,
a disappearance or reduction in number of particles, preferentially of particles larger than 25 nm, when the altering medium and/or body part is an inner region or part of a cell,
a disappearance or reduction in number of said particles, preferentially of particles smaller than 25 nm, when the particles are introduced in or to the body part of an individual for more than 1 minute,
a disappearance or reduction in number of said particles larger than 25 nm, preferentially when said particles are introduced in the body part of an individual and exposed to a radiation for more than 1 minute.

The invention also relates to the method according to the invention, wherein the alteration of step a) leads to at least one property selected from the group comprising:
a reduction in particle or nanoparticle size being of at least 10⁻⁵, 1 or 5 nm, preferentially when the pH of the altering medium and/or body part is decreased by at least 10⁻⁵, 10⁻¹ or 1 pH unit,
a disappearance or reduction in number, preferentially by a factor of more than 0.5, 1, 1.1, 2, 5 or 10, of particles of nanoparticle, preferentially of particles smaller than 10⁵, 10³, 100, 75, 50 or 25 nm, preferentially when the altering medium and/or body part has pH lower than 14, 7 or 5,
a disappearance or reduction in number, preferentially by a factor of more than 0.5, 1, 1.1, 2, 5 or 10, of particles or nanoparticles, preferentially of particles or nanoparticles larger than 10⁻³, 10⁻¹, 0, 1, 5, 25, 50, 10², 10³ or 10⁵ nm, preferentially when the altering medium and/or body part is an inner region or part of a cell,
a disappearance or reduction in number, preferentially by a factor of more than 0.5, 1, 1.1, 2, 5 or 10, of said particles or nanoparticles, preferentially of particles or nanoparticles smaller than 10³, 10² or 25 nm, when the particles are introduced in or to the body part of an individual for more than 1 minute, and
a disappearance or reduction in number, preferentially by a factor of more than 0.5, 1, 1.1, 2, 5 or 10, of said particles or nanoparticles, preferentially when the particles are introduced in the body part of an individual and exposed to a radiation for more than 1 minute.

In some cases, the kinetic of alteration can be: i) the speed or rate or level of alteration, preferentially during alteration, or ii) the alteration or the effects of alteration measured at at least one time of the alteration or alteration step.

In some cases, the kinetic of alteration or alteration is a reduction or decrease in particle size of more than 10⁻³, 10⁻¹, 1, 5, 10 or 10³ nm.

In some other cases, the kinetic of alteration or alteration is an increase in particle size of more than 10⁻³, 10⁻¹, 1, 5, 10 or 10³ nm.

In some cases, the kinetic of alteration or alteration occurs or takes place when the pH of the altering medium and or body part is varied, increased or decreased, by at least 10⁻⁵, 10⁻¹, 1, 5 or 10 pH units.

In still some other cases, the kinetic of alteration or alteration is the disappearance or reduction in number of particles, preferentially of small particles, in some cases of particles smaller than 10¹⁰, 10⁵, 10³, 10², 50, 20, 10, 5, 2 or 1 nm, in some other cases of particle larger than 10⁻¹⁰, 10⁻⁵, 10⁻³, 10⁻², 10⁻¹, 1, 2, 5, 10, 50, 10² or 10³ nm.

In some cases, small particles are particle that can have a superparamagnetic magnetic property.

In some cases, the magnetic property can be observed or measured or exist at a temperature larger than 0, 1, 5, 10, 50, 100, 200, 300, 500, 10³ or 10⁵ K (Kelvin).

In some other cases, the magnetic property can be observed or measured or exist at a temperature lower than 10⁵, 10³, 500, 300, 200, 100, 50, 10, 5, 1 or 0 K.

In some cases, the reduction in number of particle can be the decrease of the number of particle between before and after alteration by a factor, which is in some cases larger than 0, 1.001, 1.1, 1.5, 5, 10, 10³, 10⁵, 10¹⁰ or 10⁵⁰, which is in some other cases lower than 10⁵⁰, 10¹⁰, 10⁵, 10³, 10, 5, 2, 1.001, 1 or 0.

In some cases, the disappearance of small particle can be or be associated with the presence of small particle before alteration and the absence of small particle after alteration.

In some cases, the small particles reduce in number between before and after alteration, preferentially by a factor of at least 0, 1, 1.1, 1.5, 2, 5, 10, 10³, 10⁵ or 10¹⁰.

In some other cases, the small particles reduce in number between before and after alteration, preferentially by a factor of less than 10¹⁰, 10⁵, 10³, 10, 5, 2, 1.5, 1.1, 1 or 0.

In some cases, the kinetic of alteration or alteration occurs or takes place when the altering medium and/or body part has a pH lower than 14, 13, 12, 11, 10, 9, 8, 7, 6, 5, 4, 3, 2 or 1.

In some other cases, the kinetic of alteration or alteration occurs or takes place when the altering medium and/or body part has a pH larger than 1, 2, 3, 4, 5, 6, 7, 8, 9, 10, 11, 12, 13 or 14.

In still some other cases, the kinetic of alteration or alteration is the disappearance or reduction in number of particles, preferentially large particles, in some cases of particles larger than 10⁻¹⁰, 10⁻⁵, 10⁻³, 10⁻², 10⁻¹, 1, 2, 5, 10, 50, 10² or 10³ nm, in some other cases of particle lower than 10¹⁰, 10⁵, 10³, 10², 50, 20, 10, 5, 2 or 1 nm.

In some cases, large particles are particle that can have a ferromagnetic or ferromagnetic magnetic property.

In some cases, the disappearance of large particle can be or be associated with the presence of large particle before alteration and the absence of large particle after alteration.

In some cases, the large particles reduce in number between before and after alteration, preferentially by a factor of at least 0, 1, 1.1, 1.5, 2, 5, 10, 10³, 10⁵ or 10¹⁰.

In some other cases, the large particles reduce in number between before and after alteration, preferentially by a factor of less than 10¹⁰, 10⁵, 10³, 10, 5, 2, 1.5, 1.1, 1 or 0.

In some cases, the alteration or kinetic of alteration occurs or takes place when the altering medium and/or body part is the inner region or part of a cell.

In some cases, the alteration or kinetic of alteration occurs or takes place when the nanoparticles are introduced in or to the body part of an individual for more than 10⁻⁵⁰, 10⁻¹⁰, 10⁻⁵, 10⁻¹, 1, 5, 10, 10³ or 10⁵ minute(s).

In some other cases, the alteration or kinetic of alteration occurs or takes place when the nanoparticles are introduced in or to the body part of an individual for less than 10⁵⁰, 10¹⁰, 10⁵, 10, 1, 10⁻¹, 10⁻², 10⁻³ or 10⁻⁵ minute(s).

In some cases, the alteration or kinetic of alteration occurs or takes place when the particles are introduced in the body part of an individual and exposed to a radiation for more than 10⁻⁵⁰, 10⁻¹⁰, 10⁻⁵, 10⁻¹, 1, 5, 10, 10³ or 10⁵ minute(s).

In some other cases, alteration or the kinetic of alteration occurs or takes place when the particles are introduced in the body part of an individual and exposed to a radiation for less than 10⁵⁰, 10¹⁰, 10⁵, 10, 1, 10⁻¹, 10⁻², 10⁻³ or 10⁻⁵ minute(s).

The invention also relates to a method for obtaining an altered and disturbed particle comprising at least one of the following steps:

α) preferentially applying an alteration on at least one initial particle comprising at least one initial nanoparticle and at least one releasable initial compound, which is initially bound to said initial nanoparticle via an initial bond,
β) preferentially obtaining at least one altered particle, comprising at least one altered nanoparticle and at least one releasable altered compound, where α first partial release generates the release of a first part of altered compound during the alteration, said altered compounds being divided between:
i) a first part of altered compounds comprising altered compounds released from the altered nanoparticle, and
ii) a second part of altered compounds comprising altered compounds bound to the altered nanoparticle via an altered bond,
γ) preferentially applying a physico-chemical disturbance on said altered particle,
η) preferentially obtaining at least one altered and disturbed particle, comprising at least one altered and disturbed nanoparticle and at least one releasable altered and disturbed compound, where α second partial release generates the release of a second part of altered and disturbed compounds during physico-chemical disturbance, said altered and disturbed compounds being divided between:
i) group 1 of second part of altered and disturbed compounds comprising altered and disturbed compounds bound to the altered and disturbed nanoparticle via an altered and disturbed bond, and
ii) group 2 of second part of altered and disturbed compounds comprising altered and disturbed compounds released from the altered and disturbed nanoparticle, wherein the said initial particle, altered particle, and/or altered and disturbed particle comprise at least one active ingredient.

The present invention also relates to an altered particle preferentially obtainable by the previous steps α) and β), said altered particle comprising at least one altered nanoparticle and at least one releasable altered compound,

wherein said altered compounds are preferentially divided between:
a) a first part of altered compounds being released altered compounds from the altered nanoparticle, and
b) a second part of altered compounds being altered compounds bound via an altered bond to the altered nanoparticle,
wherein said altered particle preferentially comprises at least one active ingredient,
wherein said altered particle preferentially comprises one or more of the following features:
i) a size of the altered particle that is smaller than the size of the initial particle, preferentially by a percentage between 10⁻³% and 99.99%, where this percentage is S_{A}/S_{I} or (S_{I}−S_{A})/S_{I}, where S_{A} and S_{I} are the sizes of the altered and initial particles, respectively,
ii) a number of altered compounds bound to the altered nanoparticle, nₐ, that is smaller than the number of compounds bound to the initial nanoparticle, nᵢ, where nᵢ/nₐ is preferentially between 1 and 10¹⁰,
iii) a binding strength of least one bond between the altered compound and the altered nanoparticle, Sₐ, that is smaller than the binding strength of at least one bond between the initial compound and the initial nanoparticle, Sᵢ,
iv) a breaking of at least one bond between the altered compound and the altered nanoparticle,
v) a bond-dissociation energy between the altered compound and the altered nanoparticle, E_{da}, that is smaller than the bond-dissociation energy between the initial compound and the initial nanoparticle, E_{di},
vi) a coating thickness of the altered nanoparticle, CTₐ, that is smaller than the coating thickness of the initial nanoparticle, CTᵢ,
vii) a percentage in mass of organic material or carbon or carbonaceous material of the altered particle that is smaller than the percentage in mass of organic material or carbon or carbonaceous material of the initial particle,
viii) a cluttering of the altered compound bound to the altered nanoparticle that is smaller than the cluttering of the initial compound bound to the initial nanoparticle,
ix) a number of altered compounds N₁ₐ that prevent the release of altered compounds N₂ₐ from the altered nanoparticle that is smaller than the number of initial compounds N₁ᵢ that prevent the release of initial compounds N₂ᵢ from the initial nanoparticle,
x) at least one altered compound that is an inactivated, attenuated, or destroyed cell, part of a cell, virus, part of a virus, bacterium, and/or part of a bacterium,
xi) at least one altered compound that is a presented, processed, and/or exposed antigen or part of an antigen such as an epitope, and/or
xii) at least one altered compound that is a virus, part of virus, a bacterium, part of a bacterium, an antigen, and/or part of an antigen, which is/are bound, assembled, and/or coated with, to or on top of the altered nanoparticle.

The invention also relates to altered particle obtainable by the method, preferentially steps (x) and P) of the method, said altered particle comprising at least one altered nanoparticle and at least one releasable altered compound, said altered compounds being preferentially divided between:

a first part of altered compounds being released altered compounds from the altered nanoparticle, and
a second part of altered compounds being altered compounds bound via an altered bond to the altered nanoparticle,
wherein said altered particle comprises at least one active ingredient, said altered particle comprising one or more of the following features:
i) a size of the altered particle that is smaller than the size of the initial particle, preferentially by a percentage between 10⁻³% and 99.99%, where this percentage is preferentially S_{A}/S_{I} or (S_{I}−S_{A})/S_{I}, where S_{A} and S_{I} are the sizes of the altered and initial particles, respectively,
ii) a number of altered compounds bound to the altered nanoparticle, nₐ, that is smaller than the number of compounds bound to the initial nanoparticle, nᵢ, where nᵢ/nₐ is preferentially between 1 and 10¹⁰,
iii) a binding strength of least one bond between the altered compound and the altered nanoparticle, Sₐ, that is smaller than the binding strength of at least one bond between the initial compound and the initial nanoparticle, Sᵢ,
iv) a breaking of at least one bond between the altered compound and the altered nanoparticle,
v) a bond-dissociation energy between the altered compound and the altered nanoparticle, E_{da}, that is smaller than the bond-dissociation energy between the initial compound and the initial nanoparticle, E_{di},
vi) a coating thickness of the altered nanoparticle, CTₐ, that is smaller than the coating thickness of the initial nanoparticle, CTᵢ,
vii) a percentage in mass of organic material or carbon of the altered particle that is smaller than the percentage in mass of organic material or carbon of the initial particle,
viii) a cluttering of the altered compound bound to the altered nanoparticle that is smaller than the cluttering of the initial compound bound to the initial nanoparticle,
ix) a number of altered compounds N₁ₐ that prevent the release of altered compounds N₂ₐ from the altered nanoparticle that is smaller than the number of initial compounds N₁ᵢ that prevent the release of initial compounds N₂ᵢ from the initial nanoparticle,
x) at least one altered compound that is an inactivated, attenuated, or destroyed cell, part of a cell, virus, part of a virus, bacterium, and/or part of a bacterium,
xi) at least one altered compound that is a presented, processed, and/or exposed antigen or part of an antigen such as an epitope, and/or
xii) at least one altered compound that is a virus, part of virus, a bacterium, part of a bacterium, an antigen, and/or part of an antigen, which is/are bound, assembled, and/or coated with, to or on top of the altered nanoparticle.

In one embodiment of the invention, the inactivated, attenuated, or destroyed cell, part of a cell, virus, part of a virus, bacterium, and/or part of a bacterium, is an altered compound that is not infectious or does not cause an infectious disease preferentially after its administration to the body part or living individual and/or that is preferentially able to trigger an immune response against an infectious disease such as a viral, bacterial disease or a cancer.

In one embodiment, the altered compound that is presented, processed, and/or exposed is an antigen or an immune entity that interacts with a second immune entity such as a MHC, a T or B cell, an APC that triggers the production of antibodies against pathological cells or that destroy pathological cells.

In one embodiment, the altered compound that is/are bound, assembled, and/or coated with, to or on top of the altered nanoparticle is a compound that can be part of a reservoir of compounds being preferentially released by physico-chemical disturbance that could serve in a vaccine.

In some cases, the vaccine is preventive or pre-disease, i.e. it is a vaccine carried out before the infectious disease or infection occurs, preferentially to favor the production of antibodies against a viral/bacterial disease before the disease is occurring, preferentially to prevent the bacteria/viruses from entering the living individual preferentially in large quantity.

In some other cases, the vaccine is on or per disease, i.e. it is a vaccine carried out during the infectious disease or infection occurs, preferentially to favor the production of antibodies against viruses/bacteria when the disease is occurring, preferentially to reduce the quantity of viruses/bacteria in the living organism.

In some cases, the on-disease or per-disease vaccine can be assimilated with or act like an anti-viral drug or an antibiotic or can have some common properties with such drugs.

The present invention also relates to an altered and disturbed particle preferentially obtainable or obtained by the method according to the invention, said altered and disturbed particle comprising at least one altered and disturbed nanoparticle and at least one altered and disturbed compound,

wherein said altered and disturbed particle preferentially comprises at least one active ingredient, where the altered and disturbed compound is preferentially divided into one or more of the three following categories of compounds:
category A: altered and disturbed compounds, originating from the first part of the altered compound that is released in the first partial release and is not further released by physico-chemical disturbance from the altered and disturbed nanoparticle,
category B: a group 2 of a second part of altered and disturbed compounds, originating from the second partial release of the altered compound that is not released by alteration from the altered nanoparticle, and said group 2 of the second part of altered and disturbed compounds is further released by physico-chemical disturbance from the altered and disturbed nanoparticle,
category C: a group 1 of a second part of altered and disturbed compounds, originating from the second part of the altered compound that is not released by alteration from the altered nanoparticle, and is further not released by physico-chemical disturbance from the altered and disturbed nanoparticle,
   where said group 1 does not exist when all altered and disturbed compounds are released from the altered and disturbed nanoparticle.

The invention also relates to altered and disturbed particle obtainable by the method according to the invention, said altered and disturbed particle comprising at least one altered and disturbed nanoparticle and at least one altered and disturbed compound, wherein said altered and disturbed particle preferentially comprises at least one active ingredient, where the altered and disturbed compound is preferentially divided into one or more of the three following categories of compounds:
category A: altered and disturbed compounds, originating from the first part of the altered compound that is released in the first partial release and is not further released by physico-chemical disturbance from the altered and disturbed nanoparticle,
category B: a group 2 of a second part of altered and disturbed compounds, originating from the second partial release of the altered compound that is not released by alteration from the altered nanoparticle, and said group 2 of the second part of altered and disturbed compounds is further released by physico-chemical disturbance from the altered and disturbed nanoparticle,
category C: a group 1 of a second part of altered and disturbed compounds, originating from the second part of the altered compound that is not released by alteration from the altered nanoparticle, and is further not released by physico-chemical disturbance from the altered and disturbed nanoparticle,
   where said group 1 preferentially does not exist when all altered and disturbed compounds are released from the altered and disturbed nanoparticle.

The present invention also relates to a previously mentioned particle for use in the manufacturing of a medicament.

The invention also relates to the particle according to the invention for use in the manufacturing of a medicament or drug or medical device or composition or cosmetic or biological product or chemical product.

In some cases, the particle according to the invention can also be used in the manufacturing of: i) a medicament or drug, ii) a medical device, iii) a composition such as a medical, diagnostic, therapeutic, cosmetic composition, and/or iv) a therapeutic or diagnostic substance.

The present invention also relates to the particle according to the invention in the treatment of a disease, an infectious disease, a cancer, a tumor, an infection, a virus infection, or a bacterial infection.

The present invention also relates to a method of treatment of a disease, preferentially an infectious disease such as cancer or virus or bacterial infection, of an animal or a human being, comprising at least one of the following steps:

α) preferentially applying an alteration on at least one initial particle preferentially comprising at least one initial nanoparticle and at least one releasable initial compound, which is preferentially initially bound to said initial nanoparticle via an initial bond,
β) preferentially obtaining at least one altered particle, comprising at least one altered nanoparticle and at least one releasable altered compound, where a first partial release preferentially generates the release of a first part of altered compound during the alteration, said altered compounds being divided between:
i) a first part of altered compounds comprising altered compounds released from the altered nanoparticle, and
ii) a second part of altered compounds comprising altered compounds bound to the altered nanoparticle via an altered bond,
γ) preferentially applying a physico-chemical disturbance on said altered particle,
η) preferentially obtaining at least one altered and disturbed particle, comprising at least one altered and disturbed nanoparticle and at least one releasable altered and disturbed compound, where α second partial release generates the release of a second part of altered and disturbed compounds during physico-chemical disturbance, said altered and disturbed compounds being divided between:
i) group 1 of second part of altered and disturbed compounds comprising altered and disturbed compounds bound to the altered and disturbed nanoparticle via an altered and disturbed bond, and
ii) group 2 of second part of altered and disturbed compounds comprising altered and disturbed compounds released from the altered and disturbed nanoparticle,
wherein the said initial particle, altered particle, and/or altered and disturbed particle preferentially comprise at least one active ingredient.

The present invention also relates to a pharmaceutical composition comprising the particle according to the invention and a pharmaceutically acceptable carrier, wherein the active ingredient is preferentially a therapeutically effective amount of a medicament.

The invention also relates to a pharmaceutical composition comprising the particle according to the invention and a pharmaceutically acceptable carrier, wherein the active ingredient is preferentially a therapeutically effective amount of a medicament.

The invention also relates to the particle according to the invention and the pharmaceutical composition according to the invention, wherein the releasable compound is initially linked to or bound to or comprised in the coating and/or the central part of the said nanoparticle.

Preferably, the particle as previously defined and the pharmaceutical composition as previously defined, comprise at least one active ingredient selected from the group consisting of: i) a contrast agent, ii) a luminescent compound, iii) a drug or medicament, iv) a medical device, v) a cosmetic compound, vi) a therapeutic compound, vii) a medical compound, viii) a biological compound, ix) a diagnostic compound, x) a medical equipment or apparatus, xi) a composition, xii) a suspension, xiii) an excipient, xiv) an adjuvant, xv) a cytotoxic compound, xvi) a non-cytotoxic compound, xvii) an immunogenic compound, xviii) a non-immunogenic compound, xix) a pharmacological compound, xx) a non-pharmacological compound, xxi) a metabolic compound, xxii) a non-metabolic compound, xxiii) an antigen, xxiv) an antibody, xxv) a vaccine, xxvi) a virus, preferentially an attenuated or inactivated virus, and xxvii) a metal, preferentially a non-toxic metal, iron, silver, or gold.

The invention also relates to the particle according to the invention and the pharmaceutical composition according to the invention, wherein said active ingredient is selected from the group consisting of: i) a contrast agent, ii) a luminescent compound, iii) a drug or medicament, iv) a medical device, v) a cosmetic compound, vi) a therapeutic compound, vii) a medical compound, viii) a biological compound, ix) a diagnostic compound, x) a medical equipment or apparatus, xi) a composition, xii) a suspension, xiii) an excipient, xiv) an adjuvant, xv) a cytotoxic compound, xvi) a non-cytotoxic compound, xvii) an immunogenic compound, xviii) a non-immunogenic compound, xix) a pharmacological compound, xx) a non-pharmacological compound, xxi) a metabolic compound, and xxii) a non-metabolic compound, xxiii) an antigen or an epitope, xxiv) an antibody, xxv) a vaccine, xxvi) a virus, preferentially an attenuated or inactivated virus, and xxvii) a metal, preferentially a non-toxic metal, iron, silver, or gold, xviii) an antibiotic, xxix) a compound that is activated by being released from the nanoparticle, xxx) a compound that is activated more than once my being released more than once from the nanoparticle, and xxxi) a compound that is activated at least once by being released at least once from the nanoparticle by at least one alteration and/or physico-chemical disturbance.

Preferably, the compound, initial compound, altered compound, or altered and disturbed compound, is or comprises at least one active ingredient.

In one embodiment of the invention, the said particle, compound, bond, and/or nanoparticle comprise(s) or is/are at least one active ingredient.

In some cases, an active ingredient can be an ingredient that is activated by the method, or at least one step of the method according to the invention, i.e. it can be activated during or after the method, preferentially when or after the compound is released from the nanoparticle and preferentially diffuses towards the infected body part or preferentially activates an immune entity that acts against the infected body part.

In some other cases, an active ingredient is not activated by the method or at least one step of the method according to the invention, i.e. it can be activated before the method or without the method. This can also occur if the compound does not reach the infected body part or does not activate an immune entity against the infected body part. This can occur when the quantity of compounds released is not sufficient to trigger an effect against the infected body part or when the nature of the compound itself is not appropriate to trigger directly or indirectly an effect against the infected body part, e.g. the antigen is not sufficiently active because it has lost too much of its initial content during alteration.

In some cases, an active ingredient can be an ingredient that has an activity, preferentially a medical activity, preferentially a therapeutic or diagnostic activity, preferentially against a disease.

In some other cases, an inactive ingredient can be an ingredient that does not have an activity, preferentially a medical activity, preferentially a therapeutic or diagnostic activity, preferentially against a disease.

In some cases, the initial, altered, and/or altered and disturbed particle comprise(s) at least one active ingredient.

The invention relates to the particle according to the invention or to the pharmaceutical composition according to the invention, wherein:
the released compound such as the released altered compound or the released altered and disturbed compound is at least one active ingredient or behaves like at least one active ingredient, and/or
the non-released compound such as the initial compound, the non-released altered compound or the non-released altered and disturbed compound is not or does not behave like at least one active ingredient, and/or
the nanoparticle such as the initial nanoparticle, the altered nanoparticle, or the altered and disturbed nanoparticle is not or does not behave like at least one active ingredient,
   and/or
the bond such as the initial bond between initial compound and initial nanoparticle, the altered bond between altered nanoparticle and altered compound, the altered and disturbed bond between altered and disturbed compound and altered and disturbed nanoparticle is not or does not behave like at least one active ingredient,
   wherein the active ingredient is the active ingredient as defined in the invention.

The invention also relates to the method according to the invention and/or to the particle according to the invention and/or to the pharmaceutical composition according to the invention, wherein the nanoparticle is a magnetosome or a chemical analogue of a magnetosome or a chemically synthesized nanoparticle that has at least one property in common with a magnetosome, wherein the magnetosome or its chemical analogue preferentially inactivates, destroys, or releases a pathological cell, virus, bacterium, or part of it, preferentially repetitively, preferentially following alteration and/or physico-chemical disturbance, preferentially by being part of a vaccine or vaccination of an individual.

The invention also relates to the use of the particle according to the invention in or as: i) a contrast agent, ii) a luminescent compound, iii) a medical device, iv) a cosmetic compound or composition, v) a medical compound, vi) a biological compound, vii) a diagnostic compound, viii) a medical or diagnostic or therapeutic or surgical equipment or apparatus or tool, ix) a composition, x) a suspension, xi) an excipient, xii) an adjuvant, xiii) a cytotoxic compound, xiv) a non-cytotoxic compound, xv) an immunogenic compound, xvi) a non-immunogenic compound, xvii) a pharmacological compound, xviii) a non-pharmacological compound, xix) a metabolic compound, and/or xx) a non-metabolic compound.

The present invention also relates to the use of the previously mentioned particle in or as: i) a contrast agent, ii) a luminescent compound, iii) a medical device, iv) a cosmetic compound or composition, v) a medical compound, vi) a biological compound, vii) a diagnostic compound, viii) a medical or diagnostic or therapeutic or surgical equipment or apparatus or tool, ix) a composition, x) a suspension, xi) an excipient, xii) an adjuvant, xiii) a cytotoxic compound, xiv) a non-cytotoxic compound, xv) an immunogenic compound, xvi) a non-immunogenic compound, xvii) a pharmacological compound, xviii) a non-pharmacological compound, xix) a metabolic compound, xx) a non-metabolic compound, xxi) an antigen, xxii) an antibody, xxiii) a vaccine, and xxiv) a virus, preferentially an attenuated or inactivated virus, xxv) a metal, preferentially a non-toxic metal, iron, silver, or gold, xxvi) a pool or reserve or assembly of at least one active ingredient, and xxvii) a pool or reserve or assembly of at least one releasable active ingredient.

The invention also relates to a kit comprising at least one particle, preferentially the initial particle, of the method according to the invention and further comprising a magnet and/or a gel.

In some cases, the magnet is a magnetic field generating a constant magnetic field, an oscillating magnetic field and/or a magnetic field gradient. In some cases, the magnet can be attached at the surface of the individual, preferentially of the skin of the individual. In some cases, the magnet can be applied in a direction or with a sufficient strength or magnetic field gradient to enable maintaining the nanoparticle in the body part, preferentially the body part where the nanoparticle is administered or located, preferentially without preventing the compound from diffusing from this body part, preferentially achievable when the nanoparticle is magnetic and the compound is not magnetic.

In some other cases, the gel can be a material with a viscosity that is larger than the viscosity of water, or a material that maintains the nanoparticle in the body part, preferentially the body part where the nanoparticles are administered, preferentially the(a) non-infected body part. In some cases, the gel can be a material with a sufficient viscosity or specific composition that enables maintaining the nanoparticle in the body part preferentially without preventing the compound from diffusing from this body part.

In some cases, the gel and/or magnet can be located in the body part, preferentially the non-infected body part, or at distance from the body part smaller than 10²⁰, 10¹⁰, 10, 10⁵, 10³, 10, 5, 2 or 1 nm.

In some other cases, the gel and/or magnet can be located at a distance from the body part larger than 1, 5, 10, 10³, 10⁵, 10⁹, 10¹⁰ or 10²⁰ nm.

In still some other cases, the particle and/or magnet and/or gel and/or kit can be comprised in a patch, preferentially an intradermal patch or in a medical device or system that enables the release, preferentially the continuous or controlled, preferentially trough alteration or physico-chemical disturbance, of the compound from the nanoparticle.

In another embodiment, the invention relates to the use of the kit for a controlled release of the compound, preferentially through alteration and/or physico-chemical disturbance, wherein the magnet or gel is preferentially keeping the nanoparticle at the injection site and the compound is released over time.

In still another embodiment of the invention, when an entity such as the compound, substance, particle, bond, nanoparticle, radiation, has a property with a value of P₁ that is higher, longer, or larger by a factor α than a property, preferentially of this entity, with a value of P₂, it means that: P₁=α·P₂ (α preferentially larger than 1) or P₁=α+P₂ (α preferentially larger than 0).

In still another embodiment of the invention, when an entity such as the compound, substance, particle, bond, nanoparticle, radiation, has a property with a value of P₁ that is lower, smaller, or shorter by a factor β than a property, preferentially of this entity, with a value of P₂, it means that: P₁=β·P₂ (β preferentially smaller than 1), P₁=P2/β (β preferentially larger than 1), P₁=P₂−β (β preferentially larger than 0) or P₁=β−P₂.

In some cases, P1, P2, α·P₂, α+P₂, β·P₂, P₂/β, P₂−β, and/or β−P₂ can be or designate the absolute values of P1, P2, α·P₂, α+P₂, β·P₂, P₂/β, P₂−β, and/or β−P₂.

The invention will be further described by the following non-limiting figures and examples.

### DESCRIPTION OF THE FIGURES

FIG. 1: For chains of magnetosomes extracted from magnetotactic bacteria (CM), size histogram, (a), TEM microscopic image, (b), and percentage of endotoxin released following one MS, (c). For HCl treated magnetosomes, size histogram, (d), TEM image, (e), and percentage of endotoxin released following one MS, (f). For chemically synthesized iron oxide nanoparticles (IONP), size histogram, (g), TEM image (h), and percentage of endotoxins released following one MS, (i). The MS consisted in the application of an AMF of 200 kHz and 27 mT during 30 minutes.

FIG. 2: (a), (b), TEM images and associated size histogram of U87-Luc cells incubated with magnetosomes during 24 hours. In (b), magnetosomes are internalized in a cell, within an intracellular vesicle. (c), Percentage of iron internalized in U87-Luc cells, when these cells were brought into contact with 700 μg of CM or IONP during 24 hours, and either not subjected to one MS (W/o MS) or subjected to one MS (with MS). (d), Percentage of living cells measured following a treatment in which U87-Luc cells were brought into contact with 700 μg/mL of IONP or CM during 24 hours and either no exposed to one MS or exposed to one MS, MS parameters are the same as those of the legend of FIG. 1.

FIG. 3: For mice having received glucose without MS, or with 3 or 15 MS, variations of tumor BLI following the day of tumor cell implantation (0 corresponding to D-8), (a), temperature variation measured during each MS, (b), survival rate following the day of tumor cell implantation, (c). For mice having received IONP without MS or with 3 or 15 MS, variations of tumor BLI following the day of tumor cell implantation, (a), temperature variation measured during each MS, (b), survival rate following the day of tumor cell implantation, (c).

FIG. 4: For mice having received CM without MS, or with 3 or 15 MS, variations of tumor BLI following the day of tumor cell implantation, (a), temperature variation measured during each MS, (b), survival rate following the day of tumor cell implantation, (c). In the inset of (c), two representative histological images of a brain slide of a mouse euthanized 250 days following tumor cell implantation. Both show an absence of tumor. One image shows the presence of CM while the other one lacks CM.

FIG. 5: (a), Scanning electron microscopic images of a brain section collected at D30 from a mouse treated by CM administration, showing cells and CM. (b), Magnetosome size distribution deduced from (a).

FIG. 6: (a), Scanning electron microscopic images of a brain section collected from a mouse treated by CM administration followed by 15 MS at D30, where each MS consisted in the application of an AMF of 200 kHz and 27 mT applied during 30 minutes, showing cells and CM. (b), Magnetosome size distribution deduced from (a).

FIG. 7: Schematic diagram showing a method for increasing the release of at least one compound initially bound to at least one nanoparticle, wherein said compound and/or said nanoparticle contain at least one active ingredient (M), said method comprising the steps of:

alteration of said nanoparticle and said compound leading to a size reduction of said nanoparticle, preferentially without reducing the size of said compound, and leading to the weakening or breaking of said bond between said compound and said nanoparticle,
first partial release of a first part of said compound(s) from said nanoparticle, for which said bond between the said compound and the said nanoparticle is entirely broken, the second part of said altered compound is the compound(s) still weakly bound to said nanoparticle,
irradiation of said degraded and size-reduced nanoparticle and of said second part of the degraded compound(s) remaining at the surface of said degraded and size-reduced nanoparticle,
second partial release of said second part of the degraded and irradiated compound(s),
the total percentage of release of the compounds being higher when performing steps 1) and 2), preferentially successively, than only performing step 3).

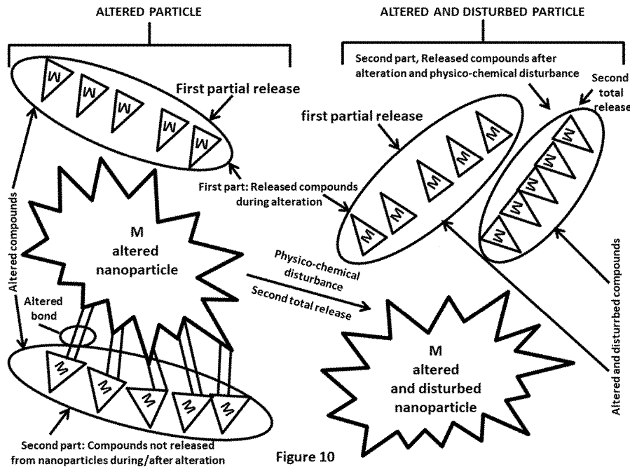

In FIG. 7) before step 1) or 3) the compounds are strongly, preferentially covalently, bound to the nanoparticle being preferentially a magnetosome (e.g. Fe₃O₄ or Fe₃S₄). This strong, preferentially covalent, binding is shown with 3 bonds.

In FIG. 7) the compounds are either released from the nanoparticle at step 1) (first partial release) or the compounds are still linked to the nanoparticle via a week link at step 3) (shown with one bond instead of three bonds).

In FIG. 7) at step 2) the remaining linked compounds are either released from the nanoparticle (second partial release) or a minority of said remaining linked compounds are still linked to the nanoparticle via a week link (shown with one bond instead of three bonds).

The technical effect of the alteration (step 1) is to weaken the bond between the altered nanoparticle and the altered compound in order to render the irradiation more efficient at step 2, i.e. increase the release of the compounds containing the active ingredient from the nanoparticles.

The radiation step 3 leads to a low release of compounds, while the successive combination of steps 1 and 2 leads to a high release of compounds.

FIG. 8: Schematic diagram showing the method for obtaining an altered particle comprising at least one of the following steps: α) applying an alteration on at least one initial particle comprising at least one initial nanoparticle and at least one releasable initial compound, which is initially bound to said initial nanoparticle via an initial bond, β) obtaining at least one altered particle, comprising at least one altered nanoparticle and at least one releasable altered compound, where a first partial release generates the release of a first part of altered compound during the alteration, said altered compounds being divided between: i) a first part of altered compounds comprising altered compounds released from the altered nanoparticle, and ii) a second part of altered compounds comprising altered compounds bound to the altered nanoparticle via an altered bond, wherein the said initial particle, altered particle, and/or altered and disturbed particle comprise at least one active ingredient (M). The initial bond is represented by three bonds and the altered bond is represented by two bonds only.

FIG. 9: Schematic diagram showing the method for obtaining an altered and disturbed particle comprising at least one of the following steps: γ) applying a physico-chemical disturbance on said altered particle, η) obtaining at least one altered and disturbed particle, comprising at least one altered and disturbed nanoparticle and at least one releasable altered and disturbed compound, where a second partial release generates the release of a second part of altered and disturbed compounds during physico-chemical disturbance, said altered and disturbed compounds being divided between: i) group 1 of second part of altered and disturbed compounds comprising altered and disturbed compounds bound to the altered and disturbed nanoparticle via an altered and disturbed bond, and ii) group 2 of second part of altered and disturbed compounds comprising altered and disturbed compounds released from the altered and disturbed nanoparticle, wherein the said initial particle, altered particle, and/or altered and disturbed particle comprise at least one active ingredient (M). The altered bond is represented by two bonds and the altered and disturbed bond is represented by one bond only.

FIG. 10: Schematic diagram showing the method for obtaining an altered and disturbed particle comprising at least one of the following steps: γ) applying a physico-chemical disturbance on said altered particle, η) obtaining at least one altered and disturbed particle, comprising at least one altered and disturbed nanoparticle and 100% of released altered and disturbed compound, when all altered and disturbed compounds are released from the altered and disturbed nanoparticle (i.e. the group 1 does not exist in this embodiment). The altered bond is represented by two bonds and the bound was entirely broken (no more shown) for released altered and disturbed compounds divided in two categories: a first category of altered compounds, originating from the first partial release of the altered compound and is not further released by physico-chemical disturbance from the altered and disturbed nanoparticle, and a second category of altered and disturbed compounds, originating from the second total release of the altered and disturbed compounds. (M) being the active ingredient. The altered bond is represented by two bonds. The released altered and disturbed compounds do no have any more bond associated to nanoparticle. The second total release represents 100% of the second part of altered compounds not released during the alteration step but which were released during/after the physico-chemical disturbance step.

### EXAMPLES

Nano-therapies against cancer led to encouraging results, notably in the treatment of glioblastoma by magnetic hyperthermia. In order to be optimal, such treatments necessitate that nanoparticles remain for a sufficiently long time in the tumor to induce strong and persistent anti-tumor activity until full tumor disappearance. At the same time, nanoparticles also need to be eliminated. Their long term accumulation in a specific part of the organism should be avoided. Such a fine adjustment of nanoparticle bio-distribution properties can be obtained by using nanoparticles that are progressively captured and degraded by the organism. However, such behavior is often associated with a reduction in size, crystallinity, heating power, and anti-tumor efficacy of nanoparticles. To the author knowledge, it has not yet been shown that nanoparticles could maintain efficient anti-tumor activity under altering and/or size-reduced conditions.

Here, we introduce nanoparticles that remain efficient against the tumor even after their degradation in vitro and in vivo by a mechanism of release of immune-stimulant substances under the application of an alternating magnetic field (AMF). Due to the use of gram negative magnetotactic bacteria to synthesize them, these nanoparticles, called magnetosomes, are made of a mineral iron oxide core surrounded by a layer consisting of biological material, mainly consisting of lipids, proteins, and endotoxins. To determine if endotoxin release is modified under conditions of alteration, such mechanism was studied for two types of magnetosome suspensions, which were either untreated or partly dissolved by being mixed with a solution of HCl. Furthermore, to examine the effect of cellular internalization on magnetosome sizes and cytotoxicity, magnetosomes were brought into contact with U87-Luc glioblastoma tumor cells, followed (or not) by one magnetic session (MS). The cell viability and the size of the magnetosomes resulting from such treatment were then measured. In vivo studies were also carried out on mice bearing implanted intracranial U87-Luc glioblastoma tumors of 2 mm3, which received 40 μg of a suspension of magnetosomes followed by 3 or 15 MS. While a moderate increase in temperature was observed during the first 3 MS (0.5 to 3° C.), the temperature did not vary during the following ones. These conditions of mild temperature increase didn't prevent antitumor activity to remain persistent during the various MS, leading to total tumor disappearance among 50% of treated mice after 15 MS. To examine the possible presence of an immune response acting against the tumor and the kinetic of magnetosome degradation and/or alteration and/or size-reduction, histological and structural analyzes were carried out by optical and scanning electron microscopies on brain slices originating from mice treated with magnetosome injection followed (or not) by magnetic hyperthermia. Finally, in order to confirm or refute the hypothetic role of endotoxins in the antitumor activity, the results obtained with the magnetosome were compared with those collected under the same conditions but using chemically synthesized nanoparticles (BNF-Starch) without endotoxins instead of the magnetosomes.

Results and Discussion

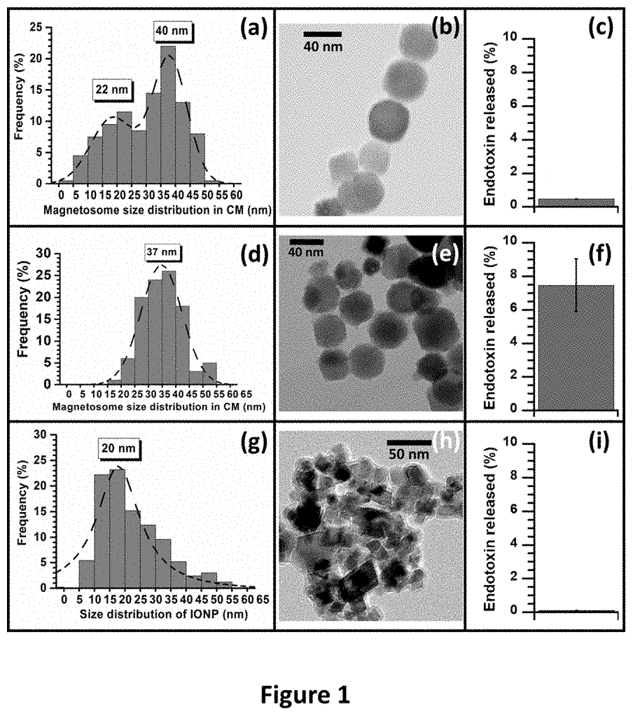

Synthesis and characterization of nanoparticles (magnetosomes) with a large endotoxin concentration. The preparation of the suspension of magnetosomes involved the following steps: i), growth of AMB-1 Magnetospirillum Magnetotacticum magnetotactic bacteria (ATCC 700264) during 7 days, ii), harvesting of a concentrated pellet of these bacteria, iii), lysis of these bacteria under sonication at 0° C. during 2 hours at 30 W, iv), isolation of magnetosome chains (CM) from cellular organic debris using a magnet, v), re-suspension of CM in a sterile injectable solution containing 5% of glucose, and vi), partial sterilization of the CM suspension by exposing CM suspension to UV irradiation for 12 h. To determine the composition of CM, infra-red absorption measurements were first carried out on a lyophilized suspension of CM, which was denatured and solubilized with KBr. The infra-red absorption spectrum of CM displays the following features: i), Amide I and Amide II bands due to protein absorption at 1650 cm⁻¹ and 1530 cm⁻¹, ii), absorption bands due to lipopolysaccharide (LPS) or phospholipids contained in the magnetosome membrane at 1050 cm⁻¹ and 1250 cm⁻¹, iii), a peak at 580 cm⁻¹ attributed to maghemite or magnetite. These results suggest a magnetosome composition consisting of an iron oxide mineral core, composed of maghemite and/or magnetite, surrounded by biological material, containing endotoxins (LPS), which binds magnetosomes together, in agreement with magnetosome composition reported elsewhere. A CHNS analysis of CM further confirmed the presence of a percentage of 12% of carbonaceous material surrounding the magnetosome mineral core. Furthermore, as can be observed in the TEM image of a dried suspension of CM deposited on top of a carbon grid, magnetosomes are characterized on the one hand by an organization in chains that prevents their aggregation, and on the other hand by a bimodal size distribution with two peaks centered at 22 and 40 nm (FIG. 1(a)), resulting in nanoparticles of sufficiently large sizes to yield a ferrimagnetic behavior at room temperature, i.e. with HC (coercivity) ˜20 mT and Mr/MS (ratio between remanent and saturating magnetization) ˜0.3. The stability of CM in suspension, which is required for efficient administration, is revealed, firstly by the behavior of the potential zeta variation of this suspension as a function of pH that displays a well-defined and repeatable behavior, i.e. a decrease from 20 mV at pH 2 to −35 mV at pH 12, and secondly by the absorption of this suspension, measured at 480 nm, which decreases moderately, i.e. by less than 30%, within 20 minutes following homogenization of this suspension (data not shown). Given the high endotoxin concentration of a CM suspension, i.e. 2000 EU per mg per mL, as well as the strong magnetosome heating power, we have studied if CM could both release endotoxins and produce heat following a MS. For the heating study, 2 μl containing 40 μg of CM mixed in water were introduced in a caliper and exposed during 600 seconds to an AMF of 200 kHz and strength 20 mT to approach in vivo conditions of treatment. Interestingly, while such treatment resulted in a significant heating power, characterized by temperature increase over the whole MS (ΔT) and specific absorption rate (SAR) of ΔT˜33° C. and SAR˜57 W/gFe (table 1), it produced a moderate release of endotoxins, estimated as QD/Qi˜0.5% (FIG. 1(c) and table 2), where QD is the quantity of endotoxins contained in the supernate of a CM suspension following MS, and Qi is the quantity of endotoxin contained in a CM suspension.

Magnetosomes treated in conditions mimicking in vivo degradation and/or alteration and/or size-reduction display an enhanced faculty to release endotoxins.

In an attempt to approach in vivo conditions of magnetosome degradation and/or alteration and/or size-reduction, we have introduced 3 mg in iron of CM in a solution containing 50 mL of 10 mM HCl at pH 1. We have then sonicated this suspension during 5 minutes at 10 W, using a sonicating finger. After having left the mixture over night at 50° C., we have collected the partly dissolved magnetosomes with a magnet. As can be seen in the TEM image of FIG. 1(e) and its corresponding size histogram (FIG. 1(d)), these magnetosomes appear to have lost their chain arrangement, to be more rounded and less faceted than untreated ones, and to possess a size distribution centered at 37 nm, which is mono-modal, less broad, and contains a lower percentage of small magnetosomes as compared with untreated magnetosomes of FIG. 1(a). Magnetosomes seem to be attacked by HCl in a different way depending on their size. While HCl treatment leads to the disappearance of the smallest magnetosomes, it does not induce a large variation in size of the largest ones, but instead seems to modify their surface, as deduced from the differences in magnetosome shapes observed between FIGS. 1(b) and 1(e). Most interestingly, when magnetosomes treated with HCl are heated by the AMF in the same conditions as for CM in section 2.1, the percentage of endotoxins that they release in the supernate following one MS is estimated as QAD/QA1˜7% (FIG. 1(f) and table 2), where QAD is the quantity of endotoxins contained in the supernate of the suspension of altered and disturbed particle and QA1 is the quantity of endotoxins contained in the suspension of altered particles. QAD/QA1 is larger than the value of QD/Qi˜0.5% measured for untreated magnetosomes (FIG. 1(c)). HCl treatment may have favored the release of endotoxins from the magnetosomes. Before such treatment, endotoxins may possibly be trapped in the biologic membrane surrounding the magnetosome mineral core, while after it the membrane could be partly denatured, letting endotoxins escape more easily.

Synthesis and characterization of nanoparticles (IONP) with a low endotoxin concentration.

IONP, which are chemically synthesized nanoparticles purchased from Micromod (BNF-Starch, reference: 10-00-102), are characterized by a series of different features compared with CM. They are composed of an iron oxide core surrounded by hydroxy-methyl-starch, as deduced from the analysis of the FT-IR spectrum of IONP, which shows a peak at 607 cm⁻¹ attributed to iron oxide and two peaks at 1022 cm⁻¹ and 1150 cm⁻¹ due to starch polymer. Compared with CM, the percentage of carbonaceous material in IONP is lower at 8.5%, as revealed by CHNS measurements. Furthermore, as deduced from the TEM image of FIG. 1(g) and associated histogram (FIG. 1(h)), the majority of IONP are smaller than CM, i.e. IONP average size is 20 nm, and IONP organize in well dispersed small aggregates that differ from CM organization in chains. Despite their difference in organization and zeta potential values that are larger than those of CM for 4<pH<12, IONP appear to be sufficiently stable to be administered to mice or used for in vitro studies. Furthermore, although IONP behave ferrimagnetically at room temperature, their values of He˜10 mT and Mr/Ms˜0.15, are significantly smaller by a factor of ˜2 than those of CM. Possibly due to the low values of their hysteretic parameters, IONP heat less efficiently than CM, producing smaller temperature increase, ΔT˜4±2° C., and specific absorption rate, SAR 10±2 W/gFe (table 1), under the same heating conditions as for CM. IONP were chosen since they originate from an endotoxin free synthesis, leading to a nanoparticle suspension with a much lower endotoxin concentration than CM (0.1 EU/mg for IONP compared with 40 EU/mg for CM). In addition, less endotoxins were released from IONP than from CM following one MS, i.e. QD˜10⁻⁵ EU for IONP compared with QD˜7.7 10⁻³ EU for CM (table 2), and QD/Qi˜0.25% for IONP (FIG. 1(i)).

Magnetosomes brought into contact with U87-Luc cells internalize inside these cells, decrease in sizes, while maintaining a certain heating power and yielding cytotoxicity.

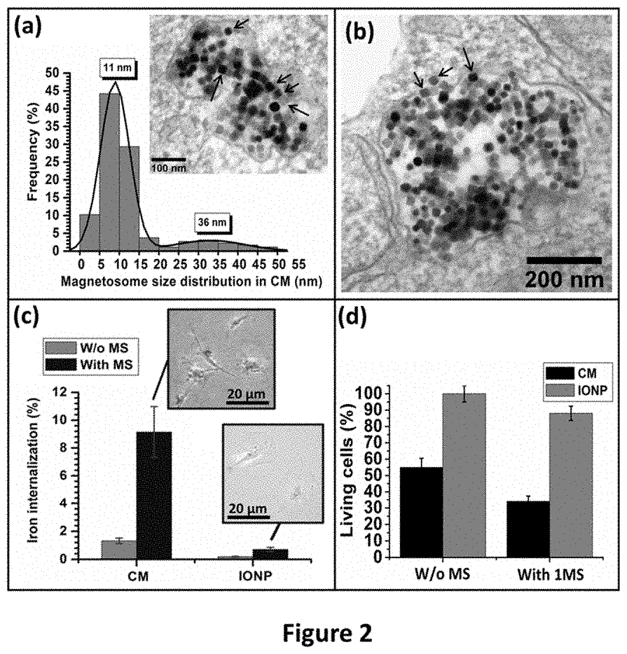

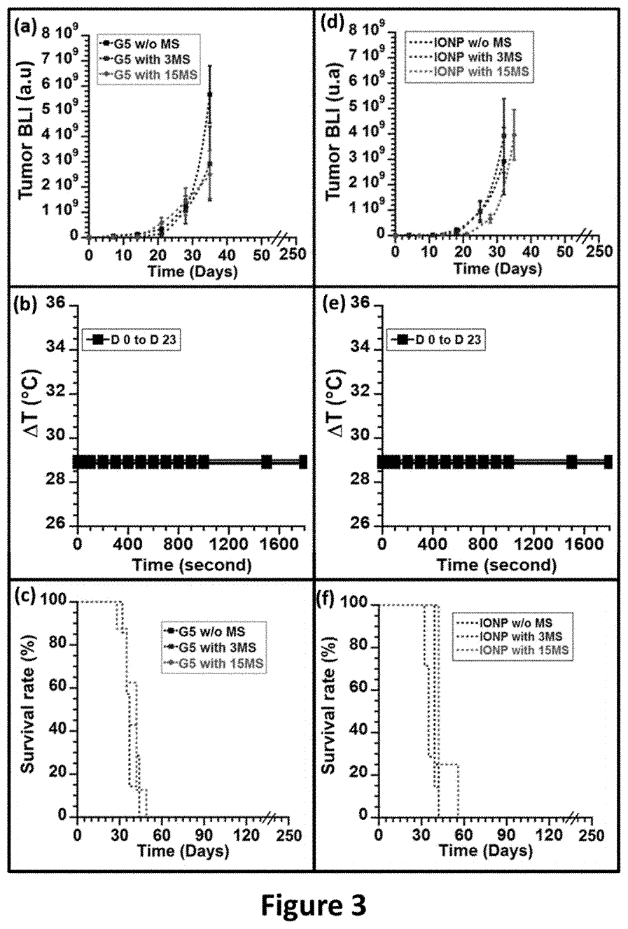

To study cellular interactions of magnetosomes in vitro, CM were incubated with U87-Luc cells during 24 hours, the cells were cut with a microtone in 80 nm thick slices, the latter were deposited on top of a carbon grid, and examined by transmission electron microscopy (TEM). Under these conditions of treatment, the TEM images of FIGS. 2(a) and 2(b), show that magnetosomes are localized inside a cell, more specifically within a cellular vesicle, which is most probably a lysosome. Compared with magnetosomes of FIG. 1(a) that are not in contact with cells, those of FIGS. 2(a) and 2(b) appear to have lost their organization in chains, to have acquired a different cubic shape, as highlighted by the black arrows designating cubic magnetosomes in FIGS. 2(a) and 2(b), and to have become smaller. Most interestingly, between before and after cellular internalization, the nanoparticle size distribution is shifted from a majority of large magnetosomes (˜40 nm, FIG. 1(a)) to a majority of small ones (˜11 nm, FIG. 2(a)). These observations indicate magnetosome cellular degradation and/or alteration and/or size-reduction, in agreement with the observations made on HCl treated magnetosomes (FIGS. 1(d) and 1(e)). Furthermore, cellular internalization appears to be enhanced following one MS. Indeed, the quantity of magnetosomes internalized inside U87-Luc cells following 24 hours of incubation increasing from 0.8% before MS to 9.2% after one MS (FIG. 2(c)). This behavior is further accompanied by a decrease in the percentage of living cells from 55% without MS to 35% with one MS (FIG. 2(d)). To understand if the efficacy of magnetosomes to destroy cells comes from intracellular heating, we have compared the properties of CM with those of IONP, where both types of nanoparticles have similar heating properties in vitro, i.e. SAR=57±11 W/gFe for CM compared with SAR=51±8 W/gFe for IONP and ΔT=7.4±0.7° C. for CM compared with ΔT=6.2±2° C. for IONP, but different internalization rates, i.e. the percentage of internalized nanoparticles varies between 0.2% (without MS) and 0.8% (with MS) for IONP and between 1.5% (without MS) and 9.2% (with MS) for CM. This last trend was confirmed by optical microscopy observations of U87-Luc cells incubated with IONP and CM during 24 hours, which were further exposed to one MS and stained with Prussian blue. Indeed, optical microscopy observations of these cells presented in the inset of FIG. 2(c) show a more persistent cyan coloration at cell location for CM than IONP, revealing the presence of a larger quantity of iron originating from nanoparticles in these cells for CM than IONP. Although these results support the idea that the cellular killing power of the magnetosomes stems from their ability to generate intracellular heating, the release of endotoxins can't be fully ruled out as another parameter responsible for in vitro magnetosome anti-tumor efficacy.

Persistent anti-tumor activity leading to full glioblastoma tumor disappearance by intra-tumor tumor administration of magnetosomes followed by AMF application even in the absence of a measured temperature increase.

Cancer thermotherapies currently in use in hospital such as high intensity focused ultrasound necessitate high heating temperatures (typically 80-90° C.) to be efficient, resulting in a number of side effects. To prevent them, the treatment can be carried out at more moderate temperatures under controlled conditions by using an external source of energy such as an AMF applied on nanoparticles contained in a tumor. Here, we therefore examine in vivo if glioblastoma can be efficiently treated when the tumor is only slightly heated during three first MS or not heated during twelve additional MS. We compare the behavior of CM with that of IONP to examine the potential roles of initial heating, endotoxin release, and cellular internalization in the anti-tumor activity.

For that, we have set-up an in vivo protocol of treatment in which 90 nude mice were divided into 9 different groups of 10 mice. We first administered 105 U87-Luc cells inside the brains of mice at the injection site (0.2.0) using a stereotactic helmet. We waited for 1 week that the tumor reached 2 mm3 and started the treatment at D0. The groups were treated as follows (Table 4):

Group 1 received at D0 2 μl of a solution of 5% of glucose at (0.2.0);
Group 2 received at D0 2 μl of 5% of glucose at (0.2.0) followed by 3 MS at D0, D1, and D2;
Group 3 received at D0 2 μl of 5% glucose at (0.2.0) followed by 12 MS at D0, D1, D2, D7, D8, D9, D14, D15, D16, D21, D22, D23;
Group 4 received at D0 2 μl of CM at (0.2.0);
Group 5 received at D0 2 μl of CM at (0.2.0) followed by 3 MS at D0, D1, D2;
Group 6 received at D0 2 μl of CM at (0.2.0) followed by 15 MS at D0, D1, D2, D7, D8, D9, D14, D15, D16, D21, D22, D23, D28, D29, D30;
Group 7 received at D0 2 μl of IONP at (0.2.0);
Group 8 received at D0 2 μl of IONP at (0.2.0) followed by 3 MS at D0, D1, D2;
Group 9 received at D0 2 μl of IONP at (0.2.0) followed by 12 MS at D0, D1, D2, D7, D8, D9, D14, D15, D16, D21, D22, D23.

Each MS (magnetic session) consisted in the application of an AMF (alternating magnetic field) of average strength 27 mT and frequency 200 kHz during 30 minutes. During the course of each MS, the temperature of the mouse brain was monitored with an infra-red camera. The size of the tumor, which was shown to be proportional to the tumor bioluminescence intensity BLI, was followed by measuring the BLI of the mouse brains during the day preceding each MS.

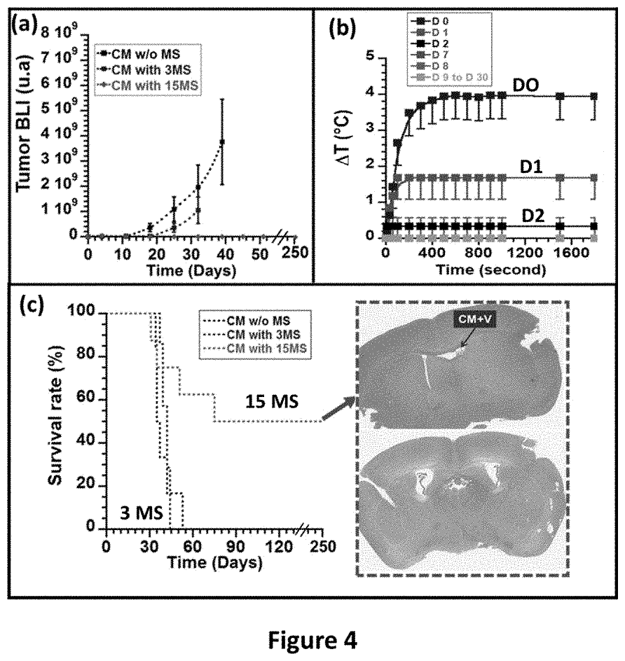

We first consider the control groups, which were not treated by nanoparticle injection followed by AMF application, i.e. those receiving glucose with/without MS (groups 1, 2, 3), or IONP/CM without MS (groups 4 and 7). In these groups, tumor BLI increased exponentially from 0 at D0 to 109-7.109 at D28 (FIGS. 3(a), 3(b), and 4(a)) and the temperature of the mouse brain remained constant during the course of each MS (FIGS. 3(b) and 3(e)). Antitumor activity did not take place and mice belonging to these groups were euthanized at D29 to D36 (FIGS. 3(c), 3(f), 4(c) and table 5), when the tumor volume exceeded 1000 mm3. Mice belonging to groups 8 and 9, which received IONP followed by 3 or 12 MS, displayed a similar behavior with an absence of any sign of efficacy or temperature increase (FIGS. 3(d) and 3(f)).

By contrast to the behaviors described above, mice treated by CM administration followed by 3 MS were prone on the one hand to a temperature increase, which was moderate and decreasing with increasing number of MS, i.e. 4, 2, and 0.5° C. during the first MS at D0, second MS at D1, and third MS at D3, respectively (FIG. 4(b)), and to a partial anti-tumor activity highlighted by the BLI decrease observed between D3 and D10 in a mouse belonging to group 5. Such partial effect was insufficient to prevent tumor re-growth after D10, which is highlighted by an exponential increase of tumor BLI between D10 and D17. Mice needed to be euthanized at D34 (FIG. 4(c) and table 5), without any improvement in median survival day compared with control groups (FIG. 4(c) and Table 5).

In order to further enhance therapeutic activity, mice belonging to group 6 were injected with CM and exposed to an additional 12 MS as compared with group 5. Under these conditions, the improvement of therapeutic activity is revealed firstly by the BLI averaged over all mice that does not increase between D0 and D242 (FIG. 4(a)), secondly by the BLI of a mouse of group 6, which continuously decreases between D4 (second MS) and D30 (fifteenth MS) and remains almost undetectable after D30, thirdly by the full tumor disappearance in 50% of mice belonging to this group, which are still alive at D242 (FIG. 4(c)), and fourthly by a mouse median survival day (MSD) above D242, which is much larger than the MSD of D27 to D38 estimated for the other groups (table 5). Furthermore, mice, which were still alive at D242, were euthanized for histological analysis. Optical micrographs of two representative brain sections of these mice are presented in FIG. 4(c), showing either some remains of magnetosomes or no sign of these nanoparticles. They are further characterized by an absence of tumor cells, lesion and edema, supporting the idea that the treatment leads to full tumor disappearance without inducing severe side effects. We concluded that although the tumor temperature stopped to increase following the third MS (FIG. 4(b)), anti-tumor activity remained strong. Furthermore, the presence of anti-tumor activity for CM and not for IONP might suggest that anti-tumor activity is only triggered in the presence of a minimum of initial temperature increase, such as that observed during the three first MS, nanoparticle cellular internalization, and/or endotoxin release. We therefore decided to study if such behavior could be due to the activation of the immune system against the tumor, possibly triggered by endotoxin release under conditions of magnetosome degradation and/or alteration and/or size-reduction.

Immune system activation by magnetosomes exposed to a magnetic session.

To examine the potential involvement of the immune system in the anti-tumor activity, we have analyzed histologically slides of mouse brains belonging to groups 1, 2, 4, 5, 7 and 8, which were collected at 6 and 72 hour following nanoparticle or glucose administration. In mice treated by injection of IONP or glucose followed (or not) by MS, the presence of other cells than tumor or healthy ones was not observed, suggesting that the immune system may not have been activated in this case. By contrast, when CM were administered in the mouse brain without MS (group 4), poly-nuclear neutrophils (PNN) were initially observed 6 hours following injection within the same region as that of CM. This behavior may be due to the release of endotoxins by magnetosomes, as observed for CM in suspension, which could attract PNN towards the magnetosomes. At 72 hours, PNN seemed to have left this region since they were not observed by histological analysis in a slide of mouse brain belonging to this group. The most interesting behavior was observed in group 5, in mice having received magnetosomes followed by MS. Indeed, the co-localization of magnetosomes and PNN was observed both at 6 hour following one MS and at 72 hours following three MS. After MS, PNN were observed to be in the proximity of the magnetosomes, suggesting that AMF application triggers a mechanism of PNN re-attraction towards magnetosomes after PNN have left the magnetosome region between 6 and 72 hours. Thus, we have highlighted a system of repeatable immune system activation by exposing nanoparticles releasing endotoxins to several AMF applications. The involvement of PNN in the destruction of the tumor is an assumption that can't be dismissed although its firm proof is difficult to establish and previous studies suggested that pro-tumor and anti-tumor activities can both be triggered by PNN.

Magnetosomes reduce in size following intra-tumor administration without losing their faculty to trigger antitumor activity.

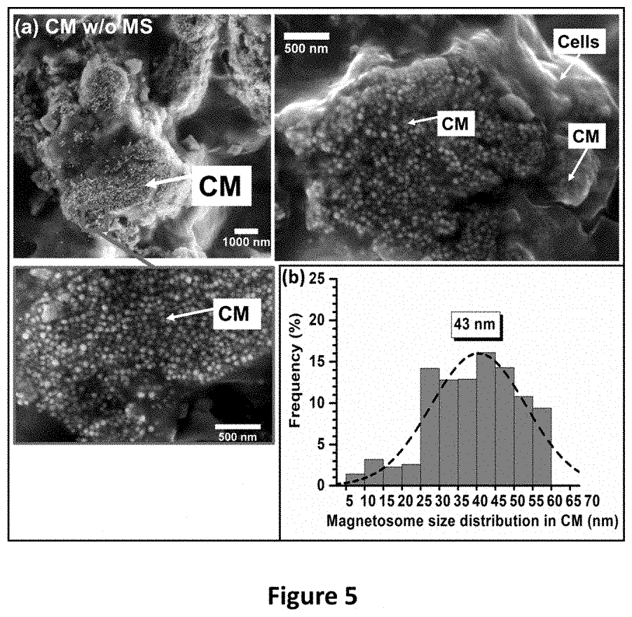

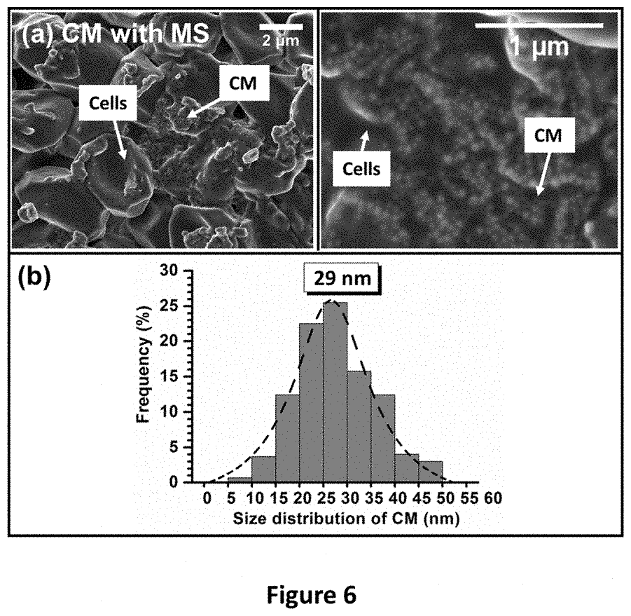

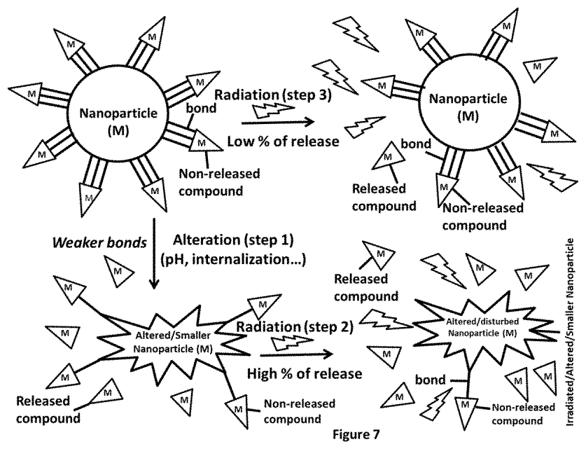

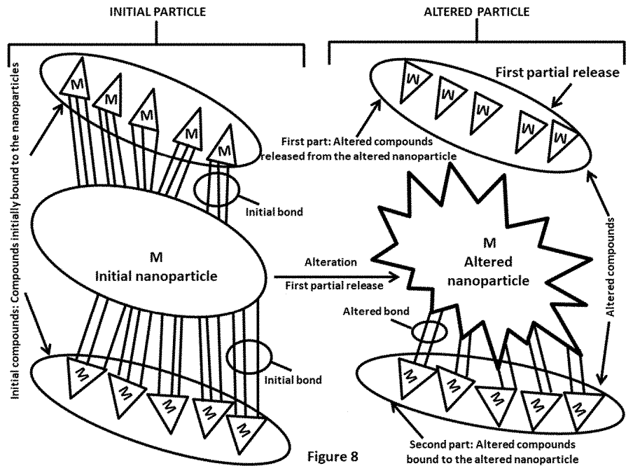

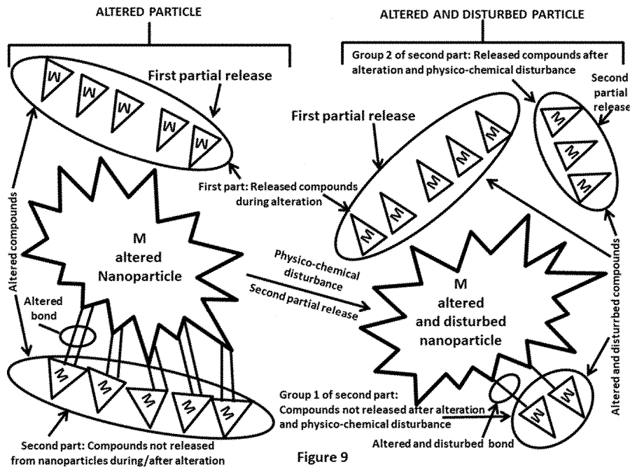

We have established that partly dissolved magnetosomes efficiently released endotoxins under AMF application. We therefore examined if such magnetosome degradation and/or alteration and/or size-reduction was taking place in vivo, since it could yield a strong immune response triggered by endotoxins. For that, slides of brains of mice bearing U87-Luc tumors treated by CM administration followed (or not) by MS were analyzed by scanning electron microscopy (SEM). In the absence of MS, the SEM image of FIG. 5(a) shows that magnetosomes are localized in the same region as cells, and are characterized by an average size of 43 nm and a mono-modal size distribution (FIG. 5(b)). Interestingly, between before and after CM administration, the magnetosome size distribution switches from bimodal to mono-modal and the magnetosomes of the smallest sizes disappear (FIGS. 1(a) and 5(b)), possibly due to magnetosomes cellular degradation and/or alteration and/or size-reduction, which could lead to full dissolution of the smallest magnetosomes of ˜22 nm (FIG. 1(a)). This behavior is consistent with that observed for magnetosomes treated with HCl. Following 15 MS, FIGS. 6(a) and 6(b) show that magnetosomes are located in the same region as cells with an average size that has decreased down to ˜29 nm, suggesting that magnetosomes have partly dissolved following the various MS, but still have a sufficiently large size to potentially induce cytotoxicity. Indeed, their size is not smaller than that of internalized magnetosomes (FIG. 2(a)) that induced efficient cellular destruction. While the decrease in size of magnetosomes leads to a loss of magnetosome heating power, i.e. the tumor temperature does not increase between the third and fifteenth MS (FIG. 4(b)), such behavior is not associated with a loss of antitumor activity, i.e. the tumor decreases in size until full disappearance between the third and fifteenth MS. Such interesting observations could be attributed to an increase of endotoxin release in degraded and/or altered and/or size-reduced magnetosomes of smaller sizes, which could activate the immune system against the tumor following AMF application.

Conclusion

Cancer nano-therapies have raised a surge of interest in the medical field due to their potential larger benefit to risk ratio compared with conventional treatments. To achieve optimal treatment outcome, nanoparticle distribution needs to be precisely controlled. On the one hand, nanoparticles should be degraded and/or altered and/or size-reduced to enable their elimination by the organism. On the other hand, such mechanism should not prevent persistent antitumor activity until full tumor disappearance. Here, we have studied nanoparticles with such desired properties, which are called magnetosome, are synthesized by magnetotactic bacteria, and are extracted from these cells for their use. Magnetosome composition consists of a ferrimagnetic iron oxide mineral core surrounded by a layer containing endotoxins, enabling both a favorable coupling between the magnetic moment of these nanoparticles and the external magnetic field and the release of endotoxins, which can potentially trigger an immunogenic reaction against the tumor. The different conditions of magnetosome degradation and/or alteration and/or size-reduction and the behaviors resulting from them were as follows:

i) When magnetosomes were treated by being mixed in solution with a HCl solution, the smallest magnetosomes were dissolved and the surface of the largest ones were degraded and/or altered and/or size-reduced, leading to a percentage of endotoxins released from magnetosomes, which increased from 0.5% to 7% between before and after treatment.
ii) Magnetosomes brought into contact with U87-Luc tumor cells internalize inside these cells, yielding a decrease of the average size of the majority of magnetosomes from ˜40 nm to ˜11 nm. Despite this decrease in sizes, magnetosomes are still able to induce ˜20% of cellular death when they are exposed to an AMF of 200 kHz and 27 mT during 20 minutes in the presence of these cells.
iii) Between before and after their administration to U87-Luc mouse tumor, the smallest magnetosomes disappear from the magnetosome size distribution that switches from bimodal (two peaks centered at ˜22 nm and ˜40 nm) before administration to mono-modal (one peak centered at ˜43 nm) after administration. An additional mouse treatment involving 15 MS, where each MS consisted in the application of an AMF of 200 kHz and 27 mT for 30 minutes, led to a further reduction in magnetosome sizes down to ˜29 nm. Despite this reduction in sizes, which resulted in a moderate temperature increase in the tumor, i.e. ˜4° C., ˜2° C., and ˜0.5° C. during the first, second, and third MS, respectively, and an absence of temperature increase between the fourth and fifteen MS, magnetosomes remained active against the tumor during the various MS until full tumor disappearance. Efficient magnetic hyperthermia in the absence of a strong temperature increase was previously reported. Here, such behavior could be explained by an immune reaction against the tumor, as suggested by the presence of poly-nuclear-neutrophils observed after one or three MS, or the release of endotoxins that is enhanced when magnetosomes are degraded and/or altered and/or size-reduced. Table 6 summarizes the effect of the different conditions of degradation on nanoparticle sizes.

### Experimental Section

Preparation of magnetosomes. To synthetize CM, we purchased magnetospirillum magneticum strain AMB-1 magnetotactic bacteria from the ATCC (700264). 4.107 of these bacteria were introduced into one liter of sterile 1653 ATCC culture medium. The media containing the bacteria were then placed in an incubator at 30° C. for 7 days to enable bacterial growth and magnetosome production. After 7 days, the media were centrifuged at 4000 g for 45 minutes. The bacterial pellet was washed using 1 ml of sterile water. Magnetotactic bacteria were concentrated using a strong Neodinium magnet (0.6 Tesla), resuspended in 0.05 M TRIS and sonicated continuously with finger at 0° C. during 2 hours at 30 W. The suspension of magnetosomes was washed several times with sterile water using a magnet to isolate magnetosome chains from the supernatant containing cellular debris and residual bacteria until cellular debris have disappeared from the supernate. Between each wash, sonication was carried out at 30 W by a series of three pulses of 2 seconds. Magnetosome chains were then resuspended in 1 mL of sterile water. For intracranial injections, magnetosome chains were resuspended in a sterile injectable solution containing 5% of glucose and exposed to irradiation of a UV lamp (UV) for 12 h for partial sterilization.

Preparation of chemically synthesized iron oxide nanoparticles (IONP). Chemically synthesized ferrimagnetic iron oxide nanoparticles (IONP) were purchased from Micromod (BNF-Starch, reference: 10-00-102. They were then centrifuged at 14,000 rpm (12×4 g) for 30 min and washed 3 times with a sterile injectable solution of 5% glucose.

TEM. To determine nanoparticle sizes, shapes, and organization, 7 μl of nanoparticle suspension were deposited on top of a carbon grid, left to dry, and then nanoparticles were then imaged using a transmission electron microscope (JEM-2100, JEOL, Japan). To obtain TEM micrographs of assemblies of cells and nanoparticles, we prepared the samples in the following manner: i), removal of culture medium from the sample containing U87 cells incubated with magnetosomes for 24 hours, ii) washing of cells with 0.2 M sodium cacodylate buffer, iii) fixing cells for 1 hour at room temperature with 2.5% glutaraldehyde in 0.2 M sodium cacodylate buffer, iv) washing 2 times the cells with 0.2 M cacodylate buffer, v) storage of cells at 4° C., vi) post-fixing of cells with osmium tetraoxide 1% and passing cells through uranyl acetate, vii) cell dehydration in an ethanol series (30%-100%), viii) embedment of cells in epoxy medium (EPON 812; Shell Chemical, San Francisco, Calif.). Ultrathin sections (80 nm) were stained by lead citrate and were examined by using a ZEISS EM902 TEM operated at 80 kV (Carl Zeiss-France, MJMA2 Microscopy Platform, UR1196, INRA, Jouy en Josas, France). Images were acquired with a charge-coupled device camera (Megaview III) and analyzed with ITEM Software (Eloise, France).

Nanoparticle characterization by absorption, CHNS, FTIR, DLS, magnetic measurements. The stability of nanoparticles in suspension was estimated by measuring the variation of the optical density of nanoparticle suspensions at 1 mg/mL in iron, measured at 480 nm, within 15 min following the homogenization of the suspension. Zeta potential of the different nanoparticles in suspension was measured by Dynamic light scattering, DLS (ZEN 3600, Malvern Instruments, UK) whose pH was adjusted between a pH 2 and 12 by using HCl and NaOH solutions. Nanoparticle FTIR spectra were recorded with a FTIR spectrometer (Vertex 70, Bruker, USA) on lyophilized nanoparticle suspensions mixed with KBr. The percentage in mass of organic material at nanoparticle surface was estimated using an elemental CHNS analyzer (Flash EA 1112, Thermo Fisher Scientific, USA). Magnetic properties of the nanoparticles were determined by measuring nanoparticle magnetization curves at room temperature between −1 and +1 T, using a vibrating sample magnetometer (VSM3900, Princeton Measurements Corporation, USA).

Measurement of the quantity of endotoxins contained in nanoparticle suspensions with the LAL assay. The endotoxin concentration was measured with the LAL assay. The latter was carried out under sterile conditions using the 88282 ThermoScientific kit called “Pierce LAL Chromogenic Endotoxin Quantitation Kit”.

Measurement of the percentage of endotoxins released by nanoparticles in suspension. 2 μl of suspensions containing 28 μg in iron of nanoparticles were introduced at the bottom of a small caliper mimicking in vivo conditions and exposed (or not) to 1 MS, during which an AMF of 202 kHz and strength 27 mT was applied during 30 minutes. The supernate was then isolated from the nanoparticles using a magnet and its endotoxin concentration was measured using the LAL assay, as described above. The percentage of endotoxins released corresponded to the ratio QAD/Qi, QAD/QA1, QD/Qi. Determination of the nanoparticle specific absorption rate. The variations of temperatures as a function of time were measured in the various treatments, during which nanoparticles in suspension, in contact with cells or in tumor brain were exposed to the AMF. The specific absorption rate was measured using the relation: SAR=(ΔT/δt)·(Cv/XFe), where Cv=4.2 J/gK is the specific heat capacity of water, XFe is the nanoparticle concentration in iron expressed in g/mL, (ΔT/δt) is the initial temperature variation with time expressed in K/sec.

Cell cultivation. Human GBM cell lines (U87-MG Luc) transduced with a Luciferase gene were cultivated in Dulbecco's Modified Eagle Medium (DMEM) containing 10% fetal bovine serum (FBS) at 37° C. in the presence of 5% CO2. After reaching confluence, culture medium was removed using Hank's Balanced Salt Solution (HBSS). Following trypsinization at 37° C. during 5 minutes, cells were detached, FBS was then added to stop the action of trypsin, and cellular concentration was measured using a Malassez counting cell.

In vitro treatment of cells. 5.10⁵ U87-Luc cells were seeded at the bottom of Petri dishes of 35 mm diameter for 24 hours. Nanoparticles of concentration in iron of 700 μg/mL were added (or not) and exposed (or not) to on MS, during which an AMF of 27 mT and 200 kHz was applied for 30 minutes. The treated assemblies of cells and nanoparticles, hence obtained, were incubated at 37° C. for an additional day. The medium containing (or not) nanoparticles was then removed and the cells were washed twice with cold PBS. Following the in vitro treatment, the percentages of living and apoptotic cells were measured using the FITC Annexin V/Dead Cell Apoptosis Kit (ThermoFisher scientific, reference: V13242). For that, 10 μL of the washed cells were loaded into the sample slide and were inserted completely into a Countess™ II FL Automated Cell Counter (Thermo Fisher scientific, reference: 15307812), which was able to detect Annexin and Propidium Iodide fluorescence emission. Following the in vitro treatments, the number of cells in the assemblies was counted by a Countess™ II FL Automated Cell Counter. Assemblies of washed cells were centrifuged, the supernatant was removed and replaced by 286 μl of HNO₃ (70%). The treated assemblies were kept at 4° C. during 24 hours to lyse cells and dissolve nanoparticles into free iron. Finally, 10 mL of filtered water were added to all treated mixtures and iron concentration was then determined using ICP-AES measurements. We deduced the average quantity of iron coming from the magnetosomes, which was internalized in each cell, using the following formula: Iron internalization (%)=100*(Q/Q°), where Q and Q° correspond to the quantity of iron internalized per cells after and before treatment, respectively. After in vitro treatment described above, cells were also stained with Prussian blue, and observed under optical microscope to examine the presence of iron, which appeared in blue color. In vivo mouse treatments. The in vivo protocol was approved by the local animal ethics committee of the University Pierre-et-Marie-Curie (Paris, France). 6 weeks old CD-1 female nude mice of average weight 20 g were purchased from Charles River. All mice were treated and kept in an environment complying with ethical guidelines and surgery was carried out following the guidelines of the Institutional Animal Care and Use Committee (“Ethic committee Charles Darwin N^{o} 5”). Mice were fed and watered according to these guidelines and we used cervical dislocation to euthanize them when their weight had decreased by more than 20% or when signs of pain, unusual posture or prostration were observed. Mice were divided in 9 groups of 10 mice. For the various treatments, the mice were anesthetized with a mixture of Ketamine (100 mg/kg) and Xylazine (8 mg/kg) in isotonic solution (0.9% of NaCl). To administer the tumor cells at D-8 and the various treatments at D0 (glucose, IONP, CM), a surgical procedure was carried out. For that, the mouse heads were fixed in a stereotactic frame, a craniotomy was realized at coordinates (0.2.0) mm and the cell suspension or various treatments (glucose, IONP, CM) were administered at (0.2.2.) mm. To follow tumor size evolution, Bioluminescence intensity (BLI) emitted by living tumor cells was measured during the day preceding each MS. We estimated that BLI maximum signal was reached 10 minutes following luciferin administration and we therefore measured BLI at that time in each mouse. A relation between tumor volume and tumor BLI was established by measuring histologically tumor volumes in a series of mice euthanized at different days following tumor cell implantation and tumor BLI in living mice at the same days as those of the euthanasia. The spatial temperature distribution in the tumor was recorded during each MS with an infrared camera (EasIRTM-2, Optophase) positioned 20 cm above the coil generating the AMF. We verified that the maximum temperature measured with the infrared camera was the same as that of the temperature measured with a thermocouple microprobe (IT-18, Physitemp, Clifton, USA) positioned at tumor center and we plotted the maximum temperature as a function of time during each MS. Mouse body weights were measured every day and mice were euthanized when losses in mouse body weights exceeded 20%. Mice, which were still alive at D250, were euthanized and brain sections were collected for further histological examination by the hematoxylin and eosin (H&E) staining to determine if the tumor had fully disappeared. Mouse survival times were plotted according to the Kaplan-Meier method. Statistical significance of survival time between the different groups was evaluated using the log rank test. Parameters were expressed as median and p-values, relative to control group.

Scanning electron microscopy (SEM) analysis of slides of mouse brains. Mouse brains were washed with 10% of sucrose, embedded in OTC (TissueTek), and kept at −80° C. in bath of isopentane cooled by liquid nitrogen. Frozen sections of their brains were obtained by cryocut (10 μm), deposited on a stub, covered by a carbon layer and analyzed by scanning electron microscope (SEM-FEG Zeiss Ultra55). We obtained surface images of cells with nanoparticles from which we could measure magnetosome sizes.

Histological analysis of slides of mouse brain. Brains were extracted from euthanized mice, fixed with a 4% solution of formaldehyde for 24 hours, cut into 2 mm thick transverse slices, washed in ethanol (70%) bath for 12 hours, and embedded in paraffin. Sections of 4 μm thick paraffin blocks were deposited on glass slides and stained with hematoxylin-eosin (H&E) to distinguish between healthy and tumor area, to determine nanoparticle location, and to examine the presence of poly-nuclear neutrophils (PNN).

Summary of experimental results:

First type of alteration: introduction of magnetosomes in a 10 mM HCl solution of pH 1.

Effect of alteration on magnetosome properties (observed for at least one magnetosome):

Organization of magnetosomes changes from an arrangement in chains before alteration to an arrangement not in chains after alteration (FIGS. 1(b) and 1(e));
Magnetosome morphology changes from cubo-octahedric before alteration to round after alteration (FIGS. 1(b) and 1(e));
Size distribution of magnetosomes changes from bimodal before alteration to mono-modal after alteration (FIGS. 1(a) and 1(d)). Magnetosomes smaller than 15 nm disappear after alteration (FIG. 1(d)).

Quantity of compounds (endotoxins) released from magnetosomes by application of a radiation, which is an AMF, increases from ˜0.5% without alteration to ˜7% with alteration (FIGS. 1(c) and 1(f)). Increase by a factor 14 of the faculty to release a compound from the magnetosomes under radiation by the alteration of the magnetosomes.

Second type of alteration: Magnetosomes brought into contact with U87-Luc cells.

Effect of alteration on magnetosome properties:

Organization of magnetosomes changes from an arrangement in chains before alteration to an arrangement not in chains after alteration (FIGS. 1(b) and 2(b));
Magnetosome morphology changes from cubo-octahedric before alteration to cubic after alteration (FIGS. 1(b) and 2(b));
Size distribution of magnetosomes changes from a distribution with a majority of large magnetosomes of average sizes 40 nm before alteration to a distribution with a majority of magnetosomes of average sizes 11 nm after alteration (FIGS. 1(a) and 2(a)).

Magnetosome anti-tumor activity is maintained after alteration. The percentage of dead cells decreases from ˜55% for magnetosomes incubated with U87-Luc cells in the absence of magnetic session to ˜35% for magnetosomes incubated with U87-Luc cells followed by one magnetic session (FIG. 2(d)).

Third type of alteration: Magnetosome administration to tumors with (or without) magnetic sessions. In the absence of magnetic session, the magnetosome size distribution changes from bimodal (two peaks centered at 22 nm and at 40 nm) before magnetosome administration to mono-modal (1 peak centered at 43 nm) after magnetosome administration (FIGS. 1(a) and 5(b)).

In the presence of magnetic sessions, the magnetosome size distribution changes from mono-modal with 1 peak centered at 43 nm 30 days after magnetosome administration (D30) without magnetic sessions to mono-modal with 1 peak centered at 29 nm at D30 following 15 magnetic sessions (FIGS. 5(b) and 6(b)). The large magnetosomes disappear following the magnetic sessions.

Despite magnetosome alteration, anti-tumor activity is persistent, which could be explained by the release of the compound (endotoxin) under altering conditions that can attract and activate the cells of the immune system against the tumor in vivo, under AMF application.

TABLE 1
Table 1: Specific absorption rate (SAR), and temperature
increase over the whole MS, ΔT, measured for: i), 2 μl or
100 μl of water containing 20 mg/mL of CM and IONP exposed to
one MS, ii), 700 μg/mL of CM and IONP incubated with U87-Luc
cells during 24 hours and exposed to one MS, iii), 2 μl containing
20 mg/mL of CM or IONP administered to 2 mm³ U87-Luc tumors
and exposed to 1 MS, 2 MS, 3 MS, 4 MS, 5 MS, 6 MS to 15 MS.
To comment the values of i), when the SAR and ΔT are measured
in a volume as small as 2 μl, which corresponds to the maximum
volume that can be administered in a mouse brain, SAR and ΔT
values are clearly underestimated compared SAR and ΔT values
measured in a larger volume (100 μl). Each MS consisted in the
application of an AMF of 200 kHz and 27 mT during 30 minutes:
Nanoparticle heating properties

| Nanoparticle | | |
|---|---|---|
| Type/condition | SAR (W/g_{Fe}) | ΔT (° C.) |
| CM (in 2 μL suspension) | 57 ± 6 | 33 ± 3 |
| 20 mg/mL in iron | | |
| IONP (in 2 μL suspension) | 10 ± 2 | 4 ± 2 |
| 20 mg/mL in iron | | |
| CM (in 100 μL suspension) | 1234 ± 307 | 95 ± 8 |
| 20 mg/mL in iron | | |
| IONP (in 100 μL suspension) | 58 ± 3 | 62 ± 5 |
| 20 mg/mL in iron | | |
| CM (in vitro, 2 mL) | 57 ± 11 | 7.4 ± 0.7 |
| 700 μg/mL in iron | | |
| IONP (in vitro, 2 mL) | 51 ± 8 | 6.6 ± 2 |
| 700 μg/mL in iron | | |
| CM (in vivo) 1 MS | 4.7 ± 1.5 | 4 ± 1 |
| 2 μL, 20 mg/mL in iron | | |
| CM (in vivo) 2 MS | 2.5 ± 1 | 1.7 ± 1 |
| 2 μL, 20 mg/mL in iron | | |
| CM (in vivo) 3 MS | 2 10⁻³ ± 1 10⁻⁴ | 0.4 ± 0.4 |
| 2 μL, 20 mg/mL in iron | | |
| CM (in vivo) 4 MS | 2 10⁻³ ± 1 10⁻⁴ | 0.4 ± 0.4 |
| 2 μL, 20 mg/mL in iron | | |
| CM (in vivo) 5 MS | 2 10⁻³ ± 1 10⁻⁴ | 0.4 ± 0.4 |
| 2 μL, 20 mg/mL in iron | | |
| CM (in vivo) 6 MS to 15 MS | 0 | 0 |
| 2 μL, 20 mg/mL in iron | | |
| IONP (in vivo) 1 MS to 12 MS | 0 | 0 |
| 2 μL, 20 mg/mL in iron | | |

TABLE 2
For a suspension of CM (chains of magnetosomes extracted from magnetotactic bacteria) or
IONP (chemically synthesized nanoparticles), quantity of endotoxins in initial nanoparticle suspension
before alteration by HCl treatment, Qi, quantity of endotoxins in suspensions of altered nanoparticles
after alteration by HCl treatment, QA1, quantity of endotoxins removed from initial nanoparticles by
alteration, QA2 = Qi − QA1, quantity of endotoxins in supernate of suspension of disturbed
nanoparticle, QD, quantity of endotoxins in supernate of suspension of altered and disturbed
nanoparticles, QAD, where the application of the physico-chemical disturbance consists in the
application of one MS. Percentage of endotoxin release for CM exposed to alteration and/or physico-
chemical disturbance, QA2/Qi (alteration step), QAD/Q1 (physico-chemical disturbance step).
Percentage of endotoxin release for CM exposed to physico-chemical disturbance, QD/Qi. Percentage
of endotoxin release for IONP exposed to physico-chemical disturbance, QD/Qi. The MS consists in
the application of an AMF of 200 kHz and 27 mT during 30 minutes.
Percentage of endotoxins released by nanoparticles in suspension
following a magnetic hyperthermia session

| | | | | | Quantity of | |
|---|---|---|---|---|---|---|
| | | | Quantity of | Quanity of | endotoxins in | |
| | Quantity of | Quantity of | endotoxins | endoxotins in | supernate of | |
| Steps of the | endotoxins | endotoxins in | removed from | supernate of | suspension of | |
| method | in initial | suspension | intial | suspension of | altered and | |
| followed | nanoparticle | of altered | nanoparticle | disturbed | disturbed | Percentage of |
| (alteration: A) | suspension | nanoparticle | by alteration | nanoparticle | nanoparticle | endotoxin |
| (Disturbance: D) | (Q [symbol] ) | (QA₂) | (QA₂) | (Q [symbol] ) | (Q [symbol] ) | release |
| A + D (CM) | 1.6 EU | [symbol] EU | ( [symbol] ) = | N.A | 6.6 10⁻³ EU | [symbol] = 94% |
| | | | 1.5 EU | | | (A step) |
| | | | | | | [symbol] = 7% |
| | | | | | | (D step) |
| D (CM) | 1.6 EU | N.A. | N.A. | 7.7 10 [symbol] EU | N.A. | Q [symbol] /Q [symbol] = 0.5% |
| D (IONP) | 4.10⁻³ EU | N.A. | N.A. | 1.0 10 [symbol] EU | N.A. | Q [symbol] /Q [symbol] = 0.25% |

[symbol]

indicates data missing or illegible when filed

Endotoxins=compounds
QA2/Qi: For CM, percentage of endotoxin release after the alteration step relatively to the quantity of endotoxins in the initial particle;
QAD/QA1: For CM, percentage of endotoxin release after alteration and physico-chemical disturbance steps relatively to the quantity of endotoxins in the altered particle;
QD/Qi: For CM or IONP, percentage of endotoxin release after physico-chemical disturbance step relatively to the quantity of endotoxins in the initial particle (no alteration step is performed in this case).

The method of the present invention shows that:

For CM exposed to the altered and physico-chemical disturbance steps, a release percentage of endotoxins (altered compound) from the altered nanoparticle of 94% when performing the alteration step, followed by a release percentage of endotoxins from the altered and disturbed nanoparticle of 7% of the 94% when performing the physico-chemical disturbance step, as illustrated in FIGS. 7 to 10. For CM exposed to the physico-chemical disturbance step, a release percentage of endotoxins (compounds) of 0.5% when performing the physico-chemical disturbance step (one MS) in the absence of alteration.

For IONP exposed to the physico-chemical disturbance step, a release percentage of endotoxins (compounds) of 0.25% when performing the physico-chemical disturbance step (one MS) in the absence of alteration.

TABLE 3
Table 3: Percentages of stability of 1 mg/mL of CM and IONP
mixed in water, measured by estimating the decrease in
absorption at 480 nm of these suspensions within 20 minutes.
Thickness of the coating of CM and IONP. Size of IONP and
CM. Iselectric point of CM and IONP suspension.

| | CM | IONP |
|---|---|---|
| % Stability on water | 70 | 100 |
| ([Fe] = 1 mg/mL) | | |
| Coating thickness (nm) | 1-5 | 1-4 |
| Size (nm) | 22 and 40 | 20 |
| Isoelectric point (pH) | 4.2 | 9.5 |

TABLE 4
Table 4: Treatment conditions of the different groups of mice.

| | D | | | | | | | | | | | | | | | | |
|---|---|---|---|---|---|---|---|---|---|---|---|---|---|---|---|---|---|
| | −8 | 0 | 1 | 2 | 7 | 8 | 9 | 14 | 15 | 16 | 21 | 22 | 23 | 28 | 29 | 30 | |
| | Days | | | | | | | | | | | | | | | | |
| | 0 | 8 | 9 | 10 | 15 | 16 | 17 | 22 | 23 | 24 | 29 | 30 | 31 | 36 | 37 | 38 | |
| Group 1 | Injection | G5 | | | | | | | | | euthanasia of mice | | | | | | The end by |
| Group 2 | of | G5 +H | +H | +H | | | | | | | | | | euthanasia of mice | | | hyperthermia |
| Group 3 | U87-Luc | G5 +H | +H | +H | +H | +H | +H | +H | +H | +H | +H | +H | +H | euthanaisa of mice | | | treatment |
| Group 4 | cells | CM | | | | | | | | | | | | euthanaisa of mice | | | |
| Group 5 | | CM +H | +H | +H | | | | | | | | | | euthanaisa of mice | | | |
| Group 6 | | CM +H | +H | +H | +H | +H | +H | +H | +H | +H | +H | +H | +H | +H | +H | +H | |
| Group 7 | | IONP | | | | | | | | | | | | | | | |

| Group 8 | IONP +H | +H | +H | | | | | | | euthanaisa of mice | | | |
|---|---|---|---|---|---|---|---|---|---|---|---|---|---|
| Group 9 | IONP +H | +H | +H | +H | +H | +H | +H | +H | +H | +H | +H | +H | euthanaisa of mice |

+H => Applications of alternating magnetic field (27 mT, 202 kHz, 30 min)
CM => Injection of chain of magnetosome
IONP => Injection of IONP
G5 => Infection of isotonic solution (5% of glucose)
Euthanasia of mice (weight decreased of 20%)
Bioluminescence measurement days: 7, 14, 21, 28, 35, 39, 45, 51, 59, 150 and 250

TABLE 5
Table 5: Median survival day and associated p-value
estimated for the different groups of treated mice.

| | Treatment | Median Survival day | | p-value |
|---|---|---|---|---|
| Group 1 | G5 | 37 | (D 29) | |
| Group 2 | G5 + 3 MS | 37 | (D 29) | 0.591 |
| Group 3 | G5 + 15 MS | 42 | (D 36) | 0.263 |
| Group 4 | CM | 36 | (D 28) | 0.552 |
| Group 5 | CM + 3 MS | 42 | (D 34) | 0.069 |
| Group 6 | CM + 15 MS | 250 | (D 242) | 0.001 |
| Group 7 | IONP | 39 | (D 31) | 0.072 |
| Group 8 | IONP + 3 MS | 35 | (D 27) | 0.480 |
| Group 9 | IONP + 12 MS | 48 | (D 38) | 0.005 |

TABLE 6
Properties of magnetosomes after and before degradation, where the properties are: i) the average size
measured over the whole size distribution (Pt) or over different peaks of the size distribution (P1
or P2), ii) the FWHM of the whole size distribution (Pt) or of the different peaks of the size distribution
(P1 or P2), iii) minimum nanoparticle size of the whole size distribution (Min), and iv) maximum nanoparticle
size of the whole size distribution (Max). The magnetosomes are either not degraded (condition before
degradation), or degraded (condition after degradation) in the following conditions: i) the suspensions
of magnetosomes are mixed with a 10 mH HCl solution of pH 1, ii) the magnetosomes are brought into contact
with U87-Luc cells, iii) the magnetosomes are administered to mouse tumors without AMF application,
iv) the magnetosomes are administered to mouse tumors with AMF applications.

| BEFORE DEGRADATION | | | | | AFTER DEGRADATION | | | | | |
|---|---|---|---|---|---|---|---|---|---|---|
| Distri. | Av | FWHM | Min | Max | Condition of | Distrib. | Av | FWHM | Min | Max |
| type | (nm) | (nm) | (nm) | (nm) | degradation | type | (nm) | (nm) | (nm) | (nm) |
| Bi- | 17.5 (P1) | 20 (P1) | 2.5 | 5.5 | Suspension of | Mono- | 37 | 20 | 15 | 55 |
| model | 37.5 (P2 | 15 (P2) | | | magnetosomes | model | | | | |
| | dominant) | 35 (Pt) | | | mixed with HCl | | | | | |
| | 27.5 (Pt) | | | | Magnetosomes | Bi- | 11 (P1 | 9 (P1) | 2.5 | 50 |
| | | | | | brought into contact | model | dominant) | 17.5 (P2) | | |
| | | | | | with U87-Luc cells | | 36 (P2) | 26.5 (Pt) | | |
| | | | | | | | 23.5 (Pt) | | | |
| | | | | | Magnetosomes | Mono- | 43 | 30 | 5 | 60 |
| | | | | | administered to | model | | | | |
| | | | | | mouse tumors | | | | | |
| | | | | | Without AMF | | | | | |
| | | | | | application | | | | | |
| | | | | | Magnetosomes | Mono- | 29 | 17.5 | 5 | 50 |
| | | | | | administered to | model | | | | |
| | | | | | mouse tumours with | | | | | |
| | | | | | AMF application | | | | | |

Nanoparticle average sized of the nanoparticle size distrubtion = Av
Full width half maximum of the nanoparticle size distribution = FWHM
The Av and FWHM measured for each individual peak of the size distribution (P1, P2 . . .) as well as for the whole size distribution (Pt)
Maximum size of the nanoparticle whole size distribution = Max
Minimum size of the nanoparticle whole size distribution = Min

Conclusion: The previous examples show an unexpected and surprising increase of compounds that are able to treat cancer by nano-therapy.

1-15. (canceled)

16. A method for increasing the release of at least one compound, said compound being initially an initial compound bound to at least one initial nanoparticle, said initial compound bound to said initial nanoparticle forming at least one initial particle,
and wherein said initial particle comprises at least one active ingredient,
and wherein said method comprises the following two steps a) and b):
a) altering said initial particle, wherein said altering is associated with modification of at least one property of said initial particle, said altering resulting in formation of an altered particle composed of at least one altered nanoparticle and at least one altered compound,
and wherein said altered particle comprises at least one active ingredient,
and wherein said altering is defined as at least one step selected in the group consisting of steps i) to xii):
i) decreasing particle size from the size of the initial particle down to the size of the altered particle, where this decrease is such that S_{A}/S_{I} or (S_{I}−S_{A})/S_{I} is between 10⁻³% and 99.99%, where S_{A} and Sᵢ are the sizes of the altered and initial particles, respectively,
ii) decreasing a number of compounds bound to the nanoparticle, from a number nᵢ of initial compounds bound to the initial nanoparticle down to a number nₐ of altered compounds bound to the altered nanoparticle, where nᵢ/nₐ is between 1 and 10¹⁰,
iii) decreasing a binding strength of least one bond between the compound and the nanoparticle, from a binding strength Sᵢ of at least one initial bond between the initial compound and the initial nanoparticle to a binding strength Sₐ of at least one altered bond between the altered compound and the altered nanoparticle,
iv) breaking at least one bond between the altered compound and the altered nanoparticle,
v) decreasing a bond-dissociation energy between the compound and the nanoparticle, from a bond-dissociation energy E_{di} between the initial compound and the initial nanoparticle down to a bond-dissociation energy E_{da} between the altered compound and the altered nanoparticle,
vi) decreasing a coating thickness of the nanoparticle, from a coating thickness CTᵢ of the initial nanoparticle down to a coating thickness CTₐ of the altered nanoparticle,
vii) decreasing a percentage in mass of organic material or carbon or carbonaceous material of the altered particle, compared with the percentage in mass of organic material or carbon or carbonaceous of the initial particle,
viii) decreasing cluttering of the compound bound to the nanoparticle, from a large cluttering of the initial compound bound to the initial nanoparticle down to a small cluttering of the altered compound bound to the altered nanoparticle,
ix) decreasing a number or a concentration of compounds N₁ that prevent the release of compounds N₂ from the nanoparticle, from a number of initial compounds N₁ᵢ that prevent the release of initial compounds N₂ᵢ from the initial nanoparticle down to a number of altered compounds N₁ₐ that prevent the release of altered compounds N₂ₐ from the altered nanoparticle,
x) inactivating, attenuating, destroying a cell, part of a cell, a virus, part of a virus, a bacterium, and/or part of a bacterium, from an initial cell, part of an initial cell, an initial virus, part of an initial virus, an initial bacterium, and/or part of an initial bacterium that is/are not inactivated, not attenuated, and/or not destroyed by or in the presence of the initial nanoparticle to an altered cell, part of an altered cell, an altered virus, part of an altered virus, an altered bacterium, and/or part of an altered bacterium that is/are inactivated, attenuated, and/or destroyed by or in the presence of the altered nanoparticle or of the nanoparticle that transforms itself from the initial to the altered nanoparticle,
xi) presenting, processing and/or exposing an antigen or part of an antigen such as an epitope by or in the presence of the altered nanoparticle, from an initial antigen or part of an initial antigen that is not presented, not processed and/or not exposed by or in the presence of the initial nanoparticle to an altered antigen or part of an altered antigen that is presented, processed and/or exposed by or in the presence of the altered nanoparticle, and
xii) coating, binding, and/or assembling a nanoparticle by or with a cell, part of a cell, a virus, part of a virus, a bacterium, part of a bacterium, an antigen, and/or part of an antigen, from an initial nanoparticle that is not coated, bound, and/or assembled by or with an initial cell, part of an initial cell, an initial virus, part of an initial virus, an initial bacterium, part of an initial bacterium, an initial antigen, and/or part of an initial antigen to an altered nanoparticle that is coated, bound, and/or assembled by or with an altered cell, part of an altered cell, an altered virus, part of an altered virus, an altered bacterium, part of an altered bacterium, an altered antigen, and/or part of an altered antigen,
and wherein said alteration results in xiii) and xiv):
xiii) a first partial release generating a first part of altered compounds released from the altered nanoparticle, where the first partial release is due to the complete breaking of the bond between said altered nanoparticle and the first part of said altered compound,
and
xiv) an absence of release generating a second part of altered compounds that remain bound to the altered nanoparticle, where the absence of release is due to the absence of breaking of the altered bond between said altered nanoparticle and said second part of altered compound,
and wherein the first part and second part of altered compounds originate from the initial particle, and the sum of said first part and second part of said altered compounds represent the total number of initial compounds bound to said initial nanoparticle,
and wherein said alteration, which is applied on the first transforming particle transforming from the initial particle to the altered particle, is carried out in at least one of the following conditions among xv) to xx): xv) by a first internalization of the first transforming particle in a cell, a virus, a bacterium, xvi) by a first variation of the pH of the first transforming particle or of its environment, xvii) by a first variation of temperature of the first transforming particle or of its environment, xviii) by bringing the first transforming particle in the presence of altering biological or chemical material, xix) by applying a first radiation on the first transforming particle, and xx) by a first variation the environment of the first transforming particle,
b) by applying a physico-chemical disturbance on said altered particle, resulting in the formation of an altered and disturbed particle,
and wherein said altered and disturbed particle comprises at least one active ingredient,
and wherein step b) is associated with xxv), xxvi), or xxvii):
xxv) an absence of release generating non-released altered and disturbed compounds belonging to group 1 of the second part, which originates from the second part of altered compounds not released by alteration at step a)xiii), wherein the absence of release is due to the absence of breaking of the altered and disturbed bond between the altered and disturbed nanoparticle and the group 1 of the second part of altered and disturbed compounds,
xxvi) a second partial release generating released altered and disturbed compounds belonging to group 2 of the second part, which originates from the second part of altered compounds not released by alteration at step a)xiii), wherein said second partial release is due to the complete breaking of the bond between the group 2 of the second part of the altered and disturbed compounds and the altered and disturbed nanoparticle,
or
xxvii) a second total release of altered and disturbed compounds, which originate from the second part of altered compounds not released by alteration at step a)xi), wherein said second total release is due to the complete breaking of the bond between all said altered and disturbed compounds and said altered and disturbed nanoparticle,
and wherein said physico-chemical disturbance which is applied on the second transforming particle transforming from the altered particle to the altered and disturbed particle, is carried out in at least one of the following conditions among xxviii) to xxxiv): xxviii) by a second internalization of the second transforming particle in a cell, a virus, a bacterium, xxix) by a second variation of the pH of the second transforming particle or of its environment, xxx) by a second variation of temperature of the second transforming particle or of its environment, xxxi) by bringing the second transforming particle in the presence of an altering biological or chemical material, xxxii) by applying a second radiation on the second transforming particle, and xxxiii) by a second variation the environment of the second transforming particle.

17. The method according to claim 16, wherein the alteration of step a) is repeated a number of time Nₐ, wherein each alteration lasts for a time t_{A}, wherein the physico-chemical disturbance of step b) is repeated a number of time N_{b}, wherein each physico-chemical disturbance lasts for a time t_{b}, wherein two different alterations are separated by a length of time tₐₐ, wherein two physico-chemical disturbances are separated by a length of time t_{bb}, wherein Nₐ N_{b}, tₐ, t_{b}, tₐₐ, and t_{bb} have at least one property selected in the group consisting of:
i) Nₐ is smaller than N_{b},
ii) Nₐ is equal to one,
iii) N_{b} is larger than one,
iv) tₐ is larger than t_{b}, and
v) t_{bb} is larger than tₐₐ.

18. The method according to claim 16, wherein the alteration, which is applied on the first transforming particle transforming from the initial particle to the altered particle, and the physico-chemical disturbance, which is applied on the second transforming particle transforming from the altered particle to the altered and disturbed particle, have at least one property selected from the group consisting of:
i) The alteration is or is due to a first variation of pH of the first transforming particle or of its environment, which is larger than 10-3 pH units,
ii) The physico-chemical disturbance is or is due to a second variation of pH of the second transforming particle or of its environment, which is larger than 10⁻³ pH units,
iii) The alteration is or is due to a first variation of temperature of the first transforming particle or of its environment, which is larger than 10⁻³° C.,
iv) The physico-chemical disturbance is or is due to a second variation of temperature of the second transforming particle or of its environment, which is larger than 10⁻³° C.,
v) The physico-chemical disturbance is associated with a second internalization of the second transforming particle, which is an extension a first internalization of the first transforming particle due to alteration,
vi) The alteration is associated with the first transforming particle being brought in the presence of altering chemical or biological material,
vii) The physico-chemical disturbance is associated with the second transforming particle being brought in the presence of altering chemical or biological material,
viii) The alteration is due to a first radiation or to the application of a first radiation on the first transforming particle,
ix) The physico-chemical disturbance is due to a second radiation or to the application of a second radiation on the second transforming particle,
and
x) The physico-chemical disturbance is a second radiation that has a strength, power, frequency, and/or intensity that is/are larger than the strength, power, frequency, and/or intensity of the first radiation being the alteration,
and wherein the altering chemical or biological material is selected in the group consisting of:
a) at least one denaturing material, where a denaturing material can be selected from a first material that induces a loss in crystallinity, activity or a reduction in size of a second material or a first material that induces unfolding such as protein unfolding or a loss in quaternary, ternary, secondary, first structure of a second material such as an enzyme or protein or a first material that induces a loss in sheet, preferentially β sheet, or helix, preferentially a helix, structures of a second material, b) at least one cell, cell organelle, protein, peptide, enzyme, DNA, RNA, DNA strand or base, RNA strand or base, part of any of these substances, preferentially denaturing, c) at least one detergent, d) at least one acid such as HCl, e) at least one base such as NaOH, f) at least one chaotropic agent, g) a compound with at least one chemical function selected in the group consisting of: carboxylic acids, phosphoric acids, sulfonic acids, esters, amides, ketones, alcohols, phenols, thiols, amines, ether, sulfides, acid anhydrides, acyl halides, amidines, nitriles, hydroperoxides, imines, aldehydes, and peroxides, h) Acetic acid, i) Alcohol, j) DMSO (Dimethylsulfoxyde), k) Ethanol, l) Formaldehyde, m) Formamide, n) Guanidine, o) Glutaraldehyde, p) Guanidinium chloride, q) Guanidine Thiocyanate, r) HCl, s) Lithium perchlorate, t) NaOH, u) Nitric Acid, v) Picric acid, w) Propylene glycol, x) Sodium bicarbonate, y) Sodium dodecyl sulfate, z) Sodium salicylate, aa) Sulfosalicylic acid, bb) Trichloroacetic acid, cc) Urea, dd) Polar solvent, ee) Apolar solvent, ff) an acidic, basic, oxidized, reduced, neutral, positively charged, negatively charged derivative of these compounds, and gg) a combination of several of these compounds or derivatives,
and wherein the first and/or second radiation(s) is/are selected in the group consisting of: a) electromagnetic radiation, b) acoustic radiation forces, c) radiation forces, d) radiation pressures, e) irradiation, preferentially of the body part, f) a source of radiation, g) a magnetic or electric field, h) an alternating magnetic or electric field, i) a magnetic or electric field gradient, j) light or laser light, k) light produced by a lamp, l) light emitted at a single wavelength, m) light emitted at multiple wavelengths, n) a ionizing radiation, o) microwave, p) radiofrequencies, q) acoustic wave, r) alpha, beta, gamma, X-ray, neutron, proton, electron, ion, neutrino, muon, meson, photon particles or radiation, s) infrasound, sound, ultra-sound, or hypersound, t) particle with a non-zero weight, and u) oscillating waves with a zero-weight.

19. A method for obtaining an altered and disturbed particle comprising at least one step selected from the group consisting of:
a) applying an alteration on at least one initial particle comprising at least one initial nanoparticle and at least one releasable initial compound, which is initially bound to said initial nanoparticle via an initial bond,
b) obtaining at least one altered particle, comprising at least one altered nanoparticle and at least one releasable altered compound, where a first partial release generates the release of a first part of altered compound during the alteration, said altered compounds being divided between i) and ii):
i) a first part of altered compounds comprising altered compounds released from the altered nanoparticle,
and
ii) a second part of altered compounds comprising altered compounds bound to the altered nanoparticle via an altered bond,
c) applying a physico-chemical disturbance on said altered particle,
d) obtaining at least one altered and disturbed particle, comprising at least one altered and disturbed nanoparticle and at least one releasable altered and disturbed compound, where α second partial release generates the release of a second part of altered and disturbed compounds during physico-chemical disturbance, said altered and disturbed compounds being divided between i) and ii):
i) group 1 of second part of altered and disturbed compounds comprising altered and disturbed compounds bound to the altered and disturbed nanoparticle via an altered and disturbed bond,
and
ii) group 2 of second part of altered and disturbed compounds comprising altered and disturbed compounds released from the altered and disturbed nanoparticle, wherein the said initial particle, altered particle, and/or altered and disturbed particle comprise at least one active ingredient.

20. An altered and disturbed particle obtainable by the method of claim 19, said altered particle comprising at least one altered nanoparticle and at least one releasable altered compound, said altered compounds being divided between a) and b):
a) a first part of altered compounds being released altered compounds from the altered nanoparticle,
and
b) a second part of altered compounds being altered compounds bound via an altered bound to the altered nanoparticle,
wherein said altered particle comprises at least one active ingredient,
wherein said altered particle comprises at least one of the properties selected from the group consisting of i) to xii):
i) a size of the altered particle that is smaller than the size of the initial particle, by a percentage between 10⁻³% and 99.99%, where this percentage is S_{A}/S_{I} or (S_{I}−S_{A})/S_{I}, where S_{A} and S_{I} are the sizes of the altered and initial particles, respectively,
ii) a number of altered compounds bound to the altered nanoparticle, nₐ, that is smaller than the number of compounds bound to the initial nanoparticle, nᵢ, where nᵢ/nₐ is between 1 and 10¹⁰,
iii) a binding strength of least one bond between the altered compound and the altered nanoparticle, Sₐ, that is smaller than the binding strength of at least one bond between the initial compound and the initial nanoparticle, Sᵢ,
iv) a breaking of at least one bond between the altered compound and the altered nanoparticle,
v) a bond-dissociation energy between the altered compound and the altered nanoparticle, E_{da}, that is smaller than the bond-dissociation energy between the initial compound and the initial nanoparticle, E_{di},
vi) a coating thickness of the altered nanoparticle, CTₐ, that is smaller than the coating thickness of the initial nanoparticle, CTᵢ,
vii) a percentage in mass of organic material or carbon or carbonaceous material of the altered particle that is smaller than the percentage in mass of organic material or carbon or carbonaceous material of the initial particle,
viii) a cluttering of the altered compound bound to the altered nanoparticle that is smaller than the cluttering of the initial compound bound to the initial nanoparticle,
ix) a number of altered compounds N₁ₐ that prevent the release of altered compounds N₂ₐ from the altered nanoparticle that is smaller than the number of initial compounds N₁ᵢ that prevent the release of initial compounds N₂ᵢ from the initial nanoparticle,
x) at least one altered compound that is an inactivated, attenuated, or destroyed cell, part of a cell, virus, part of a virus, bacterium, and/or part of a bacterium,
xi) at least one altered compound that is a presented, processed, and/or exposed antigen or part of an antigen such as an epitope, and
xii) at least one altered compound that is a virus, part of virus, a bacterium, part of a bacterium, an antigen, and/or part of an antigen, which is/are bound, assembled, and/or coated with, to or on top of the altered nanoparticle.

21. The altered and disturbed particle obtainable by the method of claim 19, said altered and disturbed particle comprising at least one altered and disturbed nanoparticle and at least one altered and disturbed compound, wherein said altered and disturbed particle comprises at least one active ingredient, where the altered and disturbed compound is divided into one or more of the three following categories of compounds:
category A: altered and disturbed compounds, originating from the first part of the altered compound that is released in the first partial release and is not further released by physico-chemical disturbance from the altered and disturbed nanoparticle,
category B: a group 2 of a second part of altered and disturbed compounds, originating from the second partial release of the altered compound that is not released by alteration from the altered nanoparticle, and said group 2 of the second part of altered and disturbed compounds is further released by physico-chemical disturbance from the altered and disturbed nanoparticle,
category C: a group 1 of a second part of altered and disturbed compounds, originating from the second part of the altered compound that is not released by alteration from the altered nanoparticle,
and is further not released by physico-chemical disturbance from the altered and disturbed nanoparticle,
where said group 1 does not exist when all altered and disturbed compounds are released from the altered and disturbed nanoparticle.

22. A method of treating a disease, an infectious disease, a cancer, a tumor, an infection, a virus infection, or a bacterial infection in a subject, comprising administering to a subject in need thereof the altered and disturbed particle according to claim 20.

23. A pharmaceutical a composition comprising the altered and disturbed particle as defined in claim 20 and a pharmaceutically acceptable carrier, wherein the active ingredient is a therapeutically effective amount of a medicament.

24. The pharmaceutical composition according to claim 23, wherein said active ingredient is selected from the group comprising: i) a contrast agent, ii) a luminescent compound, iii) a drug or medicament, iv) a medical device, v) a cosmetic compound, vi) a therapeutic compound, vii) a medical compound, viii) a biological compound, ix) a diagnostic compound, x) a medical equipment or apparatus, xi) a composition, xii) a suspension, xiii) an excipient, xiv) an adjuvant, xv) a cytotoxic compound, xvi) a non-cytotoxic compound, xvii) an immunogenic compound, xviii) a non-immunogenic compound, xix) a pharmacological compound, xx) a non-pharmacological compound, xxi) a metabolic compound, xxii) a non-metabolic compound, xxiii) an antigen, xxiv) an antibody, xxv) a vaccine, xxvi) a virus, preferentially an attenuated or inactivated virus, xxvii) a metal, preferentially a non-toxic metal, iron, silver, or gold, xxviii) an antibiotic, xxix) a compound that is activated by being released from the nanoparticle, xxx) a compound that is activated more than once my being released more than once from the nanoparticle, and xxxi) a compound that is activated at least once by being released at least once from the nanoparticle by at least one alteration and/or physico-chemical disturbance.

25. The pharmaceutical composition according to claim 23, wherein:
the released compound such as the released altered compound or the released altered and disturbed compound is at least one active ingredient or behaves like at least one active ingredient,
and/or
the non-released compound such as the initial compound, the non-released altered compound or the non-released altered and disturbed compound is not or does not behave like at least one active ingredient,
and/or
the nanoparticle such as the initial nanoparticle, the altered nanoparticle, or the altered and disturbed nanoparticle is not or does not behave like at least one active ingredient,
and/or
the bond such as the initial bond between initial compound and initial nanoparticle, the altered bond between altered nanoparticle and altered compound, the altered and disturbed bond between altered and disturbed compound and altered and disturbed nanoparticle is not or does not behave like at least one active ingredient.

26. The pharmaceutical composition according to claim 23, wherein the nanoparticle is a magnetosome.

27. A kit comprising at least one particle of the method according to claim 16 and further comprising a magnet or a gel.

28. The kit according to claim 27, wherein the magnet or gel keeps the at least one initial nanoparticle at an injection site and the compound is released over time.

29. The altered and disturbed particle according to claim 20, wherein said active ingredient is selected from the group comprising: i) a contrast agent, ii) a luminescent compound, iii) a drug or medicament, iv) a medical device, v) a cosmetic compound, vi) a therapeutic compound, vii) a medical compound, viii) a biological compound, ix) a diagnostic compound, x) a medical equipment or apparatus, xi) a composition, xii) a suspension, xiii) an excipient, xiv) an adjuvant, xv) a cytotoxic compound, xvi) a non-cytotoxic compound, xvii) an immunogenic compound, xviii) a non-immunogenic compound, xix) a pharmacological compound, xx) a non-pharmacological compound, xxi) a metabolic compound, xxii) a non-metabolic compound, xxiii) an antigen, xxiv) an antibody, xxv) a vaccine, xxvi) a virus, preferentially an attenuated or inactivated virus, xxvii) a metal, preferentially a non-toxic metal, iron, silver, or gold, xxviii) an antibiotic, xxix) a compound that is activated by being released from the nanoparticle, xxx) a compound that is activated more than once my being released more than once from the nanoparticle, and xxxi) a compound that is activated at least once by being released at least once from the nanoparticle by at least one alteration and/or physico-chemical disturbance.

30. The altered and disturbed particle according to claim 20, wherein:
the released compound such as the released altered compound or the released altered and disturbed compound is at least one active ingredient or behaves like at least one active ingredient,
and/or
the non-released compound such as the initial compound, the non-released altered compound or the non-released altered and disturbed compound is not or does not behave like at least one active ingredient,
and/or
the nanoparticle such as the initial nanoparticle, the altered nanoparticle, or the altered and disturbed nanoparticle is not or does not behave like at least one active ingredient,
and/or
the bond such as the initial bond between initial compound and initial nanoparticle, the altered bond between altered nanoparticle and altered compound, the altered and disturbed bond between altered and disturbed compound and altered and disturbed nanoparticle is not or does not behave like at least one active ingredient.

31. The method according to claim 16, wherein the nanoparticle is a magnetosome.

32. The altered and disturbed particle according to claim 20, wherein the nanoparticle is a magnetosome.
